(19) United States
(12) Patent Application Publication
(10) Pub. No.: US 2025/0305055 A1
(43) Pub. Date: Oct. 2, 2025

(54) PROGNOSTIC/PREDICTIVE BREAST CANCER SIGNATURE

(71) Applicant: Cornell University, Ithaca, NY (US)

(72) Inventor: Tan A. Ince, Greenbrook, NJ (US)

(21) Appl. No.: 18/721,847

(22) PCT Filed: Dec. 22, 2022
(86) PCT No.: PCT/US2022/082286

§ 371 Date: Jun. 19, 2024

Related U.S. Application Data
(60) Provisional application No. 63/292,943, filed on Dec. 22, 2021.

(51) Int. Cl. C12Q 1/6886

(52) U.S. Cl. CPC C12Q 1/6886; C12Q 2600/106; C12Q 2600/158

(57) ABSTRACT

Accurate methods for detecting cancer and for determining the prognosis of cancer, including breast cancer, are described herein, using biomarkers referred to herein as the ET-9 and ET-60 biomarkers. For example, ZNF92 is shown to be surprisingly specific for breast cancer. Methods for treating cancer patients classified as having a poor prognosis by the methods herein are also described herein.

### CROSS-REFERENCE TO RELATED APPLICATIONS

This application claims the benefit of the filing date of U.S. application No. 63/292,943, filed Dec. 22, 2021, the disclosure of which is incorproated by reference herein.

### INCORPORATION BY REFERENCE OF SEQUENCE LISTING

A Sequence Listing is provided herewith as an xml file, “2296015.xml” created on Dec. 20, 2022 and having a size of 112,752 bytes. The content of the xml file is incorporated by reference herein in its entirety.

### BACKGROUND

In 2021, breast cancer became the most common cancer globally, accounting for 12% of all new annual cancer cases worldwide, according to the World Health Organization. About one in eight (about 13%) of women in the U.S. will develop invasive breast cancer over the course of her lifetime. In 2021, an estimated 281,550 new cases of invasive breast cancer are expected to be diagnosed in women in the U.S., along with 49,290 new cases of non-invasive (in situ) breast cancer.

Breast cancer is the second leading cause of cancer deaths in women, with more than 40,000 deaths annually. Improved detection and prognostic methods can significantly improve the outlook for women diagnosed with breast cancer.

### SUMMARY

As illustrated herein, ZNF92, a generally unexplored transcription factor, is a marker for cancer, including breast cancer. Surprisingly, the extraordinary breast cancer specific over-expression of ZNF92, which is nearly as specific for breast cancer as the estrogen receptor (ER), has not been recognized before. Breast cancer gene expression signatures are also described herein that are referred to herein as ET-9 and ET-60, and which unlike most commercially available signatures, are independent of patient age, ethnicity, race, disease stage, metastasis, and radiation therapy, cellular proliferation, tumor subtype and lymph mode metastasis. The high expression of ET-9 and ET-60 signatures are driven by histone deacetylase 7 (HDAC7) and ZNF92.

The ET-9 signature, for example, can predict significantly shorter (8.7 years) overall survival (p=0.0001) and 6.26 years shorter relapse free survival (p=006). The results described herein indicate that the ET-9 and ET-60 signatures are prognostic tests for breast cancer, useful to identify patients with poor outcome, hereby allowing those patients to be treated with additional cycles or combinations of therapies. In addition, ET-9 and ET-60 can be used as a predictive signature to select patients for HDAC inhibitor treatment.

Described herein are methods that can include: (a) assaying a biological sample from a subject for expression of ZNF92, ET-9 biomarkers recited in Table 1, or nine or more of the FT-60 biomarkers recited in Table 2 to determine one or more expression levels for the ZNF92, ET-9, or nine or more of the ET-60 biomarkers; (b) comparing the determined expression levels with one or more reference values to identify any altered expression levels in the subject's biological sample, wherein altered expression levels of the ZNF92, ET-9, or nine or more of the ET-60 biomarkers in the biological sample relative to the reference value indicates that the subject has cancer with poor prognosis or the subject has malignant cancer, and absence of altered expression of the ZNF92 ET-9, or nine or more of the ET-60 biomarkers relative to the reference value indicates that the subject does not have a cancer with poor prognosis or does not have malignant cancer; and (c) administering one or more histone deacetylase inhibitors, ZNF92 inhibitors, histone demethylase inhibitors, mTOR inhibitors, polo-like kinase (PLK) inhibitors, heat shock factor inhibitors, or a combination thereof to a subject determined to have a cancer with poor prognosis or a malignant cancer. In one embodiment, the amount of (level of expression of) RNA encoding a polypeptide having SEQ ID NO:1 or a polypeptide having at least 80%, 82%, 85%, 87%, 88%, 89%, 90%, 92%, 94%, 95%, 97%, 98% or 99% amino acid sequence identity thereto, or a portion thereof, in a sample is determined.

In one embodiment, the amount of RNA encoding a polypeptide having at least two of SEQ ID Ns. 3-11 or a polypeptide having at least 80%, 82%, 85%, 87%, 88%, 89%, 90%, 92%, 94%, 95%, 97%, 98% or 99% amino acid sequence identity thereto, or a portion thereof, is determined. In one embodiment, the amount of RNA encoding a polypeptide having at least two of SEQ ID Ns. ³-11 or a polypeptide having at least 80%, 82%, 85%, 87%, 88%, 89%, 90%, 92%, 94%, 95%, 97%, 98% or 99% amino acid sequence identity thereto, or a portion thereof, is determined.

In some cases the methods can include treating a subject classified as having poor cancer prognosis, comprising administering one or more histone deacetylase inhibitors, ZNF92 inhibitors, histone demethylase inhibitors, mTOR inhibitors, polo-like kinase inhibitors, heat shock factor inhibitors, or a combination thereof to the subject, wherein the subject is classified has having poor cancer prognosis by measuring expression levels of at least one sample from the subject and determining that the at least one sample has altered expression of the ZNF92, ET-9, or nine or more of the ET-60 biomarkers relative to at least one reference value.

In some cases the methods can include treating a subject having altered expression of ZNF92, ET-9 biomarkers, or nine or more of the ET-60 biomarkers relative to at least one reference value, by administering one or more histone deacetylase inhibitors, ZNF92 inhibitors, histone demethylase inhibitors, mTOR inhibitors, polo-like kinase inhibitors, heat shock factor inhibitors, or a combination thereof to the subject.

One or more reference values can be an average or median of expression levels of at least the ZNF92, ET-9, or ET-60 biomarkers in biological samples from a population of healthy subjects.

The subject can have, or be suspected of having, breast cancer, ovarian cancer, colon cancer, brain cancer, pancreatic cancer, prostate cancer, lung cancer, melanoma, leukemia, myeloma, or lymphoma.

In addition, ZNF92 can be a novel target for development of breast cancer specific treatments. For example, a method can be used for identifying a candidate agent that reduces ZNF92 expression, protein level, or activity. Such a method can include: (a) contacting ZNF92 with a test agent; (b) measuring the expression level or activity of ZNF92; and (c) determining that the test agent reduces the level or activity of ZNT92, to thereby identifying a candidate agent that reduces ZNF92 protein level or activity.

### BRIEF DESCRIPTION OF THE FIGURES

FIGS. 1A-ID. ZNF92 expression in human tumors

FIG. 1A. Gene Set Enrichment Analysis (GSEA) of FIDAC1&7 downstream targets. The top 10 pathways are depicted in the GSEA heatmap, each row represents a unique gene (Entrez ID first column), and each column represents an enriched gene set (p-value range for the top ten pathways 1.47e-11 to 6.5e-16). The blue boxes mark the 86 HDACI&7 upregulated genes that are associated with each gene set. The analysis is carried out using the online tool. The first column highlights 29 genes associated with ZNF92 binding sites in the promoter (website at www.gsea msigdb.org/gsea/msigdb/collections.jsp).

FIG. 1B. Human Protein Atlas (HPA) Pancancer expression analysis of ZNF92 (website at www.proteinatlas.org/). RNA-seg data from 17 cancer types visualized with box plots, shown as median and 25th and 75th percentiles. Points are displayed as outliers if they are above or below 1.5 times the interquartile range (website at www.proteinatlas.org!ENSG00000146757-ZNF92/pathology).

FIG. 1C. The relative mRNA expression of ZNF92, Estrogen receptor (ERSR1), HER2 (ERBB2) and MYC in the eBioportal TCGA PanCancer dataset that includes 37 tumor types with 10,967 samples (website at www.cbioportal.org/). See Tables 5-6 for the complete list of 37 tumor types. Breast cancer is the third tumor type from the left.

FIG. 1D. The relative ZNF92 mRNA expression in the tumor, normal and metastatic tissues in the TNMplot database that has RNA-seq data of TCGA including 730 normal, 9,886 tumor and 394 metastasis samples (website://tnmplot.com/analysis/).

FIGS. 2A-2F. Breast cancer specific expression of HDCAI&7 downstream targets. Human Protein Atlas (HPA) PanCancer expression analysis of SNPH (Synaflaph/lin) (FIG. 2A), CACNG4 (Calcium voltage-gated channel auxiliary subunit gamma 4) (FIG. 2B), IGFBP5 (insulin like growth.factor binding protein 5) (FIG. 2C), ZNF768 (Zinc Finger Protein 768) (FIG. 2D), BCAS4 (breast carcinoma ampdlied sequence 4) (FIG. 2E), and PR.EXI (phosphatidylinositol-3,4,5-trispho,sphate dependent Rac exchangefatzor 1) (FIG. 2F). The RNA-seq data from 17 cancer types is visualized with box plots, shown as median and 25th and 75th percentiles. Points are displayed as outliers if they are above or below 1.5 times the interquartile range (see website at www.proteinatlas.org/).

FIGS. 3A-3H: ET-60 prognostic groups compared to other signatures. Kaplan-Meier (KM) survival charts are shown of human breast cancer in the BRCA_TCGA 2016 dataset (FIGS. 3A-3D), NKI dataset (FIGS. 3E-3G) and SKI (SE12276 data set (FIG. 3H) generated using SurvExpress (see website at bioinformatica.mty.itesm.mx/SurvExpress) where high risk groups are shown by the red lines, medium risk groups are shown by green lines, and low risk groups are shown by blue lines.

FIG. 3A shows a KM survival chart of ET-60 expression in TCGA, IR: 5,76 (CI: 4.0-8.2).

FIG. 3B shows a KM survival chart of 70-gene signature in TCGA (Mammaprint); FIR: 4.73 (CIL 3.3-6.6); four genes were not found in TCGA Breast invasive carcinoma—July 2016 dataset AA555029_RC, LOC100131053, LOC100288906, LOC730018.

FIG. 3C shows a KM survival chart of 50-gene signature in TCGA (PAM50/Prosignia), HR: 3.29 (CI: 2.4-4.4); all genes found in the dataset.

FIG. 3D shows a KM survival chart of 25-gene signature (BPMS) in TCGA, HR: 2.64 (CI: 2.0-3.4). 3 Genes not found in the dataset: ZH3H3, HS3STSB1, PDECI.

FIG. 3E shows a Survival KM chart of ET-60 expression in the NK I dataset, IR: 13.39 (CI: 6.1-29.2).

FIG. 3F shows a Time to metastasis KM chart of ET-60 expression in the NK1 dataset, KR: 5.76 (CI: 3.8-8.5).

FIG. 3G shows a Time to recurrence KM chart of ET-60 expression in the NKI dataset, HR: 5.58 (CI: 3.7-8.2).

FIG. 3H shows a Time to brain relapse KM chart of ET-60 expression in the SKI dataset, HR: 9.5×10⁹.

FIGS. 4A-4D. ET-9 expression and breast cancer survival FIG. 4A shows an expression heatmap of ET-9 genes in the TCGA Breast Invasive Carcinoma mRNA (RNA Seq V2) dataset, including 1,082 patient samples. The subtype classification is provided above the heatmap; basal-like (purple) HER2+(red), Luminal A (blue), Luminal B (yellow), normal-like (green) (see website at wwivw.cbioportal.org).

FIG. 4B shows relative survival statistics of breast cancer patients with altered ET-9 expression in the TCGA (n=1,084 patients) and METABRIC (n=1,904 patients) datasets. Analysis carried out using clioPortal.

FIG. 4C shows a Kaplan-Meier plot depicting progression free survival of invasive breast carcinoma patients in the TCGA PanCancer dataset. ET-9 altered (red) tumors have significantly shorter progression free survival compared to ET-9 unaltered (blue line) tumors (p=: 0,00232). Analysis carried out using cBioPortal.

FIG. 4D shows a Kaplan-Meier plot depicting overall free survival of invasive breast carcinoma patients in the TCGA PanCancer dataset. ET-9 altered (red) tumors have significantly shorter progression free survival compared to ET-9 unaltered (blue line) tumors (p=0.000163). Analysis carried out using cBioPortal.

FIGS. 5A-5F. ET-9 prognostic groups. The Kaplan-Meier survival plots were generated using SurvExpress (see website at bioinformatica.mty.itesm.mx/SurvExpress).

FIG. 5A graphically illustrates ET-9 overall survival high risk (red), medium risk (green), low risk (blue) tumors, BRCA_TCGA 2016 dataset, HR: 3.04.

FIG. 5H graphically illustrates ET-9 metastasis high risk (red), medium risk (green), low risk (blue) tumors, NKI dataset, HR: 2.15.

FIG. 5C graphically illustrates ET-9 brain relapse high risk (red), low risk (green), GiSE12276 dataset, FIR: 10.95.

FIG. 5D graphically illustrates 21-gene Oncotype overall survival high risk (red), medium risk (green), low risk (blue) tumors, HR: 3.02.

FIG. 5E graphically illustrates 12-gene Endopredict overall survival high risk (red), medium risk (green), low risk (blue) tumors, IR: 2.29.

FIG. 5F graphically illustrates Maol2-gene signature overall survival high risk (red), medium risk (green), low risk (blue) tumors, HR: 2.05.

FIGS. 6A-6F. ET-9 prognostic groups. Kaplan-Meier survival plots generated using Kaplan-Meier plotter [Breast](see website at kmplot.com/analysis/index.php?p=service&cancer:=breast (kmplot.coin)) FIG. 6A shows Kaplan-Meier survival plots for HER2+ tumors, where ET-9 survival high risk is shown as a red line, and low risk is shown as a black line, HR: 2.27 [CI 1.45-3.55], p=2.4e-4.

FIG. 6B shows Kaplan-Meier survival plots for Triple negative (TNBC) tumors, wherein ET-9 relapse free survival high risk is shown as a red line, and low risk is shown as a black line, HR: 3.95 [CI 1.97-7.94], p=::3.i e-5.

FIG. 6C shows Kaplan-Meier survival plots for Lymph node positive tumors, where ET-9 relapse free survival high risk is shown as a red line, and low risk is shown as a black line, HR: 1.68 [CI 1.31-2.15], p=3.8e-5.

FIG. 6D shows Kaplan-Meier survival plots for Patients following systemic chemotherapy treatment, wherein ET-9 relapse free survival high risk is shown as a red line, low risk is shown as a black line, HR: 2.79 [CI 1.69-4.58], p=2.5e-5.

FIG. 6E shows Kaplan-Meier survival plots for Triple negative (TNBC) tumors, where Endopredict relapse free survival high risk is shown as a red line, and low risk is shown as a black line, HR: 1,43 [CI 0.69-2.94], p:=033.

FIG. 6F shows Kaplan-Meier survival plots for Lymph node positive tumors, where Oncotype elapse free survival high risk is shown as a red line, and low risk is shown as a black line, HR: 1.17 [CI 0.9-1.52], p=0.23.

FIGS. 7A-7F. ET-60 in breast cancer subgroups. Kaplan-leier (K M) charts of relapse free survival of human breast cancer are shown that were generated using Kaplan-Meier plotter [Breast] where high risk is shown as red lines, and low risk is shown as black lines. The analysis was carried out with user selected probe sets with auto selection for best cut off, exclusion of biased arrays, and multivariate analysis (see kmplot com/analysis/index.php?p=service&cancer=breast website).

FIG. 7A shows a KM chart of ET-60 in HER2+ human breast cancer, HR: 1.61 [CI 1.04-2.5], p=0.032.

FIG. 7B shows a KM chart of ET-60 in triple negative breast cancer (TNBC), HR: 4.19 [C1 1.5-11.66], p:0.0029.

FIG. 7C shows a KM chart of ET-60 in breast cancer patients with systemic chemotherapy, HR: 2.73 [CI 1.61-4.64], p=:0.00011.

FIG. 7D shows a KM chart of ET-60 in lymph node positive human breast cancer, HR: 1.45 [CI 1,11-1.89], p=0.0055.

FIG. 7E shows a KM chart of PAM50 (Prosignia) in triple negative breast cancer (TNBC), ER: 1.5 [CI 0.85-2.65], p=0.16.

FIG. 7F shows a KM chart of PAM50 (Prosignia) in breast cancer patients with systemic chemotherapy, HR: 1.24 [CI 0.76-2.03], p=0.38.

FIGS. 8A-8F. ET-9 (FIGS. 8A-8C) and ET-60 (FIGS. 8D-8F) prognostic groups in cervix (FIGS. 8A and 8D), utenus (FIGS. 8B and SE) and prostate cancer (FIGS. 8C and 8F). The Kaplan-Meier survival plots shown in FIG. 8 were generated using SurvExpress (see website at bioinformatica.inty.itesmn.mx/SurvExpress).

FIGS. 9A-B show that breast cancer cell line proliferation is inhibited by combination of HDAC, HSP, mTOR, polo-like kinase and Histone demethylase inhibitors.

### DETAILED DESCRIPTION

As illustrated herein, ZNF92, ET-9, and ET-60 are markers useful for detecting, diagnosing, and determining the prognosis of cancer, including breast cancer. Methods for detecting, diagnosing, and determining the prognosis of cancer, including breast cancer, are also described herein.

The methods generally involve obtaining a sample from a subject and comparing gene expression levels in the sample with one or more reference values, where the expression levels of the following genes are compared: a ZNF92 gene, ET-9 genes, ET-60 genes, or a combination of those genes. The method can also include classifying the subject from whom the sample was obtained as having cancer (i.e., being a cancer patient) or not having cancer. The method can also include classifying a cancer patient as having a poor prognosis based upon the expression levels of the ZNF92 gene, ET-9 genes, ET-60 genes, or a combination of those genes in the patient's sample. In some cases, the subject is a breast cancer patient.

For example, a method for classifying a breast cancer patient according to prognosis, can include: (a) comparing the respective levels of expression of a ZNF92 gene, of ET-9 genes, of ET-60 genes, or a combination of the genes in a sample taken from a breast cancer patient to respective reference values of expression of the genes; and (b) classifying the breast cancer patient according to prognosis of his or her breast cancer based on altered expression levels of the ZNF92, the ET-9 genes, nine or more ET-60 genes, or a combination thereof

### Samples

Breast cancer can be assessed through the evaluation of expression patterns, or profiles, of the ZNF92, ET-9, and ET-60 genes in one or more subject samples. The term subject, or subject sample, refers to an individual regardless of health and/or disease status. A subject can be a subject, a study participant, a control subject, a screening subject, or any other class of individual from whom a sample is obtained and assessed using the markers and/or methods described herein. Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. Alternatively, a subject can be healthy with respect to any of the aforementioned factors or criteria. It will be appreciated that the term “healthy” as used herein, is relative to breast cancer status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer.

In some cases, the methods for detecting, predicting, and/or assessing the prognosis of breast cancer include collecting a biological sample comprising a cell or tissue, such as a breast tissue sample or a primary breast tumor tissue sample. By “biological sample” is intended any sampling of cells, tissues, or bodily fluids in which expression of ZNF92, ET-9, or ET-60 genes can be detected. Examples of such biological samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumor tissue sample. Biological samples may be obtained from a subject by a variety of techniques including, for example, by scraping or swabbing an area, by using a needle to aspirate cells or bodily fluids, or by removing a tissue sample (i.e., biopsy). In some embodiments, a breast tissue sample is obtained by, for example, fine needle aspiration biopsy, core needle biopsy, or excisional biopsy.

The samples can be stabilized for evaluating and/or quantifying ZNF92, ET-9, or ET-60 expression levels.

In some cases, fixative and staining solutions may be applied to some of the cells or tissues for preserving the specimen and for facilitating examination. Biological samples, particularly breast tissue samples, may be transferred to a glass slide for viewing under magnification. In one embodiment, the biological sample is a formalin-fixed, paraffin-embedded breast tissue sample, particularly a primary breast tumor sample.

### Gene Expression

Various methods can be used for evaluating and/or quantifying ZNF92, ET-9, or ET-60 expression levels. By “evaluating and/or quantifying” is intended determining the quantity or presence of an RNA transcript or its expression product of ZNF92, ET-9, or ET-60 genes.

Methods for detecting expression of the ZNF92, ET-9, or ET-60 genes, including gene expression profiling, can involve methods based on hybridization analysis of polynucleotides, methods based on sequencing of polynucleotides, immunohistochemistry methods, and proteomics-based methods. The methods generally involve detect expression products (e.g., mRNA or proteins) encoding by the ZNF92, ET-9, or ET-60 genes. In some cases, PCR-based methods, which can include reverse transcription PCR (RT-PCR) (Weis et al., TIG 8:263-64, 1992), array-based methods such as microarray (Schena et al., Science 270:467-70, 1995), or combinations thereof are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to ZNF92, ET-9, or ET-60 genes. Probes can be synthesized or obtained from ZNF92, ET-9, or ET-60 nucleic acids or they can be derived from appropriate biological preparations. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a cell or tissue sample, a tumor or tumor cell line, a corresponding normal tissue or cell line, or a combination thereof. If the source of RNA is a sample from a subject, RNA (e.g., mRNA) can be extracted, for example, from stabilized, frozen or archived paraffin-embedded, or fixed (e.g., formalin-fixed) tissue samples (e.g., pathologist-guided tissue core samples). General methods for RNA extraction are available and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Jnvest. 56:A67, 1987) and De Andres et al. (Biotechniques 18:42-44, 1995). In some cases, RNA isolation can be performed using a purification kit, a buffer set and protease from commercial manufacturers, such as Qiagen (Valencia, Calif), according to the manufacturer's instructions. For example, total RNA from cells can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from tissue or cell samples (e.g. tumors) can be isolated, for example, by cesium chloride density gradient centrifugation. Additionally, large numbers of tissue samples can readily be processed using available techniques, such as, for example, the single-step RNA isolation process of Chomczynski (U.S. Pat. No. 4,843,155). Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to any of the ZNF92, ET-9, or ET-60 genes, or any derivative DN A or RNA. Hybridization of an mRNA with the probe indicates that the ZNF92, ET-9, or ET-60 genes in question is being expressed.

In cases, the mRNA from the sample is immobilized on a solid surface and contacted with a probe, for example by running the isolated mRNA on an agarose gel and transferring the rRNA from the gel to a membrane, such as nitrocellulose. In other cases, the probes are immobilized on a solid surface and the mRNA is contacted with the probes, for example, in an Agilent gene chip array. A skilled artisan can readily adapt available mRNA detection methods for use in detecting the level of expression of the ZNF92, ET-9, or ET-60 genes.

An alternative method for determining the level of ZNF92, ET-9, or ET-60 gene expression in a sample involves the process of nucleic acid amplification of the ZNF92, ET-9, or ET-60 m RNA (or cDNA thereof), for example, by RT-PCR (U.S. Pat. No. 4,683,202), ligase chain reaction (Barany, Proc. al. Natl. Acad. Sci. USA 88:189-93, 1991), self-sustained sequence replication (Guatelli et al., Proc. Natl. Acad Sci. USA 87:1874-78, 1990), transcriptional amplification system (Kwoh et al., Proc. Natl. Acad Sci. USA 86:1173-77, 1989), Q-Beta Replicase (Lizardi et al., Bio Technology 6:1197, 1988), rolling circle replication (U.S. Pat. No. 5,854,033), or any other nucleic acid amplification method, followed by the detection of the amplified molecules using available techniques. These detection schemes are especially useful for the detection of nucleic acid molecules if such molecules are present in very low numbers.

In some cases, ZNF92, ET-9, or ET-60 gene expression is assessed by quantitative RT-PCR. Numerous different PCR or QPCR protocols are available and can be directly applied or adapted for use using the ZNF92, ET-9, or ET-60 genes. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif), ABI PRISM 7700@(Applied Biosystems, Foster City, Calif), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif) and MX4000@3 (Stratagene, La Jolla, Calif).

Quantitative PCR (QPCR) (also referred as real-time PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumor blocks, such as those used in archival tissue banks and routine surgical pathology specimens (Cronin et al. (2007) Chn Chem 53:1084-91)[Mullins 2.007][Paik 2004]. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In some cases, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface can be used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

When using microarray techniques, PCR amplified inserts of cDNA clones can be applied to a substrate in a dense array. The microarrayed genes, immobilized on the microchip, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA can be hybridized pairwise to the array. The relative abundance of the transcripts from the two sources corresponding to each specified gene is thus determined simultaneously. A miniaturized scale can be used for the hybridization, which provides convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumor types.

As used herein “level”, refers to a measure of the amount of, or a concentration of a transcription product, for instance an mRNA, or a translation product, for instance a protein or polypeptide.

As used herein “activity” refers to a measure of the ability of a transcription product or a translation product to produce a biological effect or to a measure of a level of biologically active molecules.

As used herein “expression level” further refer to gene expression levels or gene activity. Gene expression can be defined as the utilization of the information contained in a gene by transcription and translation leading to the production of a gene product.

The terms “increased,” or “increase” in connection with expression of the biomarkers described herein generally means an increase by statically significant amount. For the avoidance of any doubt, the terms “increased” “increase” means an increase of at least 10% as compared to a reference value, for example an increase of at least about 20%, or at least about 30%, or at least about 40%, or at least about 50%, or at least about 60%, or at least about 70%, or at least about 80%, or at least about 90% or u to and including a 100% increase or any increase between 10-100% as compared to a reference value or level, or at least about 1-5 fold, at least about a 1,6 fold, at least about a 1.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level. in some embodiments, an increase is at least about 1.8-fold increase over a reference value.

Similarly, the terms “decrease,” or “reduced,” or “reduction,” or “inhibit” in connection with expression of the biomarkers described herein generally to refer to a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

A “reference value” is a predetermined reference level, such as an average or median of expression levels of each of ZNF92, ET-9, or ET-60 biomarkers in, for example, biological samples from a population of healthy subjects. The reference value can be an average or median of expression levels of each of ZNF92, ET-9, or ET-60 biomarkers in a chronological age group matched with the chronological age of the tested subject. In some embodiments, the reference biological samples can also be gender matched. In some embodiments, the reference biological samples can also be cancer containing tissue from a specific subgroup of patients, such as stage 1, stage 2, stage 3, or grade 1, grade 2, grade3 cancers, non-metastatic cancers, untreated cancers, hormone treatment resistant cancers, HER2 amplified cancers, triple negative cancers, estrogen negative cancers, or other relevant biological or prognostic subsets. For example, as explained herein, malignancy associated response signature expression levels in a sample can be assessed relative to normal breast tissue from the same subject or from a sample from another subject or from a repository of normal subject samples. If the expression level of a biomarker is greater or less than that of the reference or the average expression level, the biomarker expression is said to be “increased” or “decreased,” respectively, as those terms are defined herein. Exemplary analytical methods for classifying expression of a biomarker, determining a malignancy associated response signature status, and scoring of a sample for expression of a malignancy associated response signature biomarker are explained in detail herein.

### Treatment

Methods are described herein for treating cancer. Such methods can involve administering therapeutic agents that can treat cancers with poor prognosis. Examples of such therapeutic agents can include one or more histone deacetylase inhibitor, ZNF92 inhibitor, histone demethylase inhibitor, mTOR inhibitor, polo-like kinase (PLK) inhibitor, heat shock factor inhibitor, and/or inhibitors of any of the ET-9 and/or ET-60 breast cancer cell-origin associated signature biomarkers described herein.

In some cases, the cancer includes breast cancer, ovarian cancer, colon cancer, brain cancer, pancreatic cancer, prostate cancer, lung cancer, or melanoma. In some embodiments, the cancer includes leukemia, myeloma, or lymphoma.

The methods can include downregulating expression of one or more of the following: ZNF92, histone deacetylase, histone demethylase, mTOR, polo-like kinase, proteins with heat shock factors, any of the ET-9 biomarkers, any of the ET-60 biomarkers, or a combination thereof. Suitable methods for downregulating such expression can include: inhibiting transcription of mRNA; degrading mRNA by methods including, but not limited to, the use of interfering RNA (RNAi); blocking translation of mRNA by methods including, but not limited to, the use of antisense nucleic acids or ribozymes, or the like. In some embodiments, a suitable method for downregulating expression may include providing to the cancer a small interfering RNA (siRNA) targeted to ZNF92, histone deacetylase, histone demethylase, mTOR, polo-like kinase, proteins with heat shock factors, any of the ET-9 biomarkers, any of the ET-60 biomarkers, or a combination.

Suitable methods for down-regulating the function or activity of ZNF92, histone deacetylase, histone demethylase, mTOR, polo-like kinase, proteins with heat shock factors, any of the ET-9 biomarkers, any of the ET-60 biomarkers, or a combination thereof may include administering a small molecule inhibitor that inhibits the function or activity of any of these markers or factors.

In some cases, one or more histone deacetylase inhibitors can be administered to treat cancers with poor prognosis, such as cancers identified by measuring and/or monitoring ZNF92, any of the ET-9 biomarkers, and/or any of the ET-60 biomarkers described herein. In some cases, histone deacetylase inhibitors are not administered to treat cancers with poor prognosis, such as cancers identified by measuring and/or monitoring ZNF92, any of the FT-9 biomarkers, and/or any of the ET-60 biomarkers described herein As used herein a “Histone Deacetylase inhibitor” or “HDAC inhibitor” refers to inhibitors of Histone Deacetylase 1 (HDAC1), Histone Deacetylase 7 (HDAC7), and/or phosphorylated HDAC7, including agents that inhibit the level and/or activity of HDACI and/or HDAC7 and/or phosphorylated HDAC7, as well as agents that inhibit the phosphorylation of HDAC7 e.g., inhibitors of EMK protein kinase, C-TAKI protein kinase, and/or CAMK protein kinase, and agents that activate or increase the level and/or activity of phosphatase activity to remove phosphoryl groups from HDAC7, e.g., activators of PP2A phosphatase and/or myosin phosphatase. In some cases, HDAC inhibitors include molecules that bind directly to a functional region of-DACI and/or HDAC7 and/or phosphorylated HDAC7 in a manner that interferes with the enzymatic activity of HDACI and/or l-DAC7 and/or phosphorylated l-DAC7 e.g., agents that interfere with substrate binding to HDACI and/or HDAC7 and/or phosphorylated HDAC7. In some embodiments, HDAC inhibitors include molecules that bind directly to HDAC7 in a manner that prevents the phosphorylation of IDAC7. ID-AC inhibitors include agents that inhibit the activity of peptides, polypeptides, or proteins that modulate the activity of HDACI and/or HDAC7 e.g., inhibitors of EMK protein kinase, C-TAKI kinase, CAMK protein kinase inhibitors of C-TAK 1 protein kinase. Examples of suitable inhibitors include, but are not limited to antisense oligonucleotides, oligopeptides, interfering RNA e.g., small interfering RNA (siRNA), small hairpin RNA (shRNA), aptamers, ribozymes, small molecule inhibitors, or antibodies or fragments thereof, and combinations thereof.

In some cases, HDAC inhibitors are specific inhibitors or specifically inhibit the level and/or activity of HDACI and/or HDAC7 and/or phosphorylated HDAC7. As used herein, “specific inhibitor(s)” refers to inhibitors characterized by their ability to bind to with high affinity and high specificity to HDAC1 and/or HDAC7 and/or phosphorylated HDAC7 proteins or domains, motifs, or fragments thereof, or variants thereof, and preferably have little or no binding affinity for non-HDACI and/or non-HDAC7 and/or non-phosphorylated HDAC7 proteins. As used herein, “specifically inhibit(s)” refers to the ability of an HDAC inhibitor of the present invention to inhibit the level and/or activity of a target polypeptide, e.g., HDAC1, and/or HDAC7, and/or phosphorylated HDAC7, and/or EMK protein kinase, and/or C-TAK1 protein kinase and/or CAMK protein kinase and preferably have little or no inhibitory effect on non-target polypeptides. As used herein, “specifically activate(s)” and “specifically increase(s)” refers to the ability of an HDAC inhibitor of the present invention to stimulate (e.g., activate or increase) the level and/or activity of a target polypeptide, e.g., PP2A phosphatase and/or myosin phosphatase and preferably to have little or no stimulatory effect on non-target polypeptides.

Examples of HDAC inhibitors include Vorinostat (SAHA), Entinostat (MS-275), Panobinostat (L13H589), Trichostatin A (TSA), Mocetinostat (MGCD0103), 4-Phenylbutyric acid (4-PBA), ACY-775, Belinostat (PXD101), Romidepsin (FK228, Depsipeptide), MC1568, Tubastatin A 1C0, Givinostat (ITF2357), Dacinostat (LAQ824), CUDC-101, Quisinostat (JNJ-26481585) 2HCI, Pracinostat (SB939), PCI-34051, Droxinostat, Abexinostat (PCI-24781), RGFP966, AR-42, Ricolinostat (ACY-1215), Valproic Acid (NSC 93819) sodium salt, Tacedinaline (C1994), Fimepinostat (CUDC-907), Sodium butyrate, Curcumin, M344, Tubacin, RG2833 (RGFP109), Resminostat, Divalproex Sodium, Scriptaid, Sodium Phenylbutyrate, Tubastatin A, Tubastatin A TFA, Sinapinic Acid, TMP269, Santacruzamate A (CAY10683), TMP195, Valproic acid (VPA). UF010, Tasquinimod, SKLB-23bb, Isoguanosine, NKL22, Sulforaphane, BRD73954, BG45, Domatinostat (4SC-202), Citarinostat (ACY-241), Suberohydroxamic acid, BRD3308, Splitomicin, HPOB., LMK-235, Biphenyl-4-sulfonyl chloride, Nexturastat A, BML-210 (CAY10433), T^{C}-H-106, SR-4370, T134, Tucidinostat (Chidamide), SIS17, (-)-Parthenolide, WT161, CAY10603, ACY-738, Raddeanin A, GSK3117391, Tinostamustine(EDO-S101), or combinations thereof. Such HDAC inhibitors are available from Selleckchem.com.

In some cases, one or more histone demethylase inhibitors can be administered to treat cancers with poor prognosis, such as cancers identified by measuring and/or monitoring ZNF92, any of the ET-9 biomarkers, and/or any of the ET-60 biomarkers described herein. Examples of histone demethylase inhibitors include GSK-J4, 2,4-Pyridinedicarboxylic Acid, AS8351, Clorgyline hydrochloride, CPI-455, Daminozide, GSK-2879552, GSK-J1, GSK-J2, GSK-J5, GSK-L)SD1, IOXI, I0X2, IB-04, ML-324, NCGC00244536, OG-L002, ORY-1001, SP-2509, TC-E 5002, UNC-926, β-Lapachone, or combinations thereof. Such inhibitors are available, e.g., from Selleckchem.com.

In some cases, one or more m*TOR inhibitors can be administered to treat cancers with poor prognosis, such as cancers identified by measuring and/or monitoring ZNF92, any of the ET-9 biomarkers, and/or any of the ET-60 biomarkers described herein. Examples of mTOR inhibitors include Rapamycin (AY-22989), Everolimus (RAD001), AZD8055, Temsirolimus (CCI-779), PI-103, NU7441 (KU-57788), KU-0063794, Torkinib (PP242), Ridaforolimus (Deforolimus, MK-8669), Sapanisertib (MLN0128), Voxtalisib (XL765) Analogue, Torin 1, Omipalisib (GSK2126458), OSI-027, PF-04691502, Apitolisib (GDC-0980), GSK1059615, WYE-354, Gedatolisib (PKI-587), Vistusertib (AZD2014), Torin 2, WYE-125 132 (WYE-132), BGT226 (NVP-BGT226) maleate, Palomid 529 (P529), PP121, WYE-687, Clemastine (HS-592) furnarate, Nitazoxanide (NSC 697855), WAY-600, ETP-46464, GDC-0349, PI3K/Akt Inhibitor Library, 4EGI-I, XL388, MHY1485, 3-Hydroxyanthranilic acid, Bimiralisib (PQR309), Samotolisib (LY3023414), Lanatoside C, Rotundic acid, L-Leucine, Chrysophanic Acid, Voxtalisib (XL765), GZNE-477, CZ415, Astragaloside IV, CC-1 15, Salidroside, Compound 401, 3BDO, Zotarolimus (ABT-578), GNE-493, Paxalisib (GDC-0084), Onatasertib (CC 223), ABTL-s0812, PQR620, SF2523, Niclosamide, or combinations thereof. Such HDAC inhibitors are available from Selleckchem.com.

In some cases, one or more Polo-Like Kinase (PLK) inhibitors can be administered to treat cancers with poor prognosis, such as cancers identified by measuring and/or monitoring ZNF92, any of the ET-9 biomarkers, and/or any of the ET-60 biomarkers described herein. Examples of PLK inhibitors include BI 2536, Volasertib (131 6727), Wortmannin (KY 12420), Rigosertib (ON-01910), GSK461364, HMN-214, MLN0905, Ro3280, SBE 13 HCl, Centrinone (LCR-263), CFI-400945, HMN-176, Onvansertib (NMS-P937), or combinations thereof.

In some cases, one or more heat shock factor inhibitors can be administered to treat cancers with poor prognosis, such as cancers identified by measuring and/or monitoring ZNF92, any of the ET-9 biomarkers, and/or any of the ET-60 biomarkers described herein. Examples of heat shock factor inhibitors include one or more of the following Tanespimycin (17-AAG), Pimitespib (TAS-116, Luninespib (NVP-AUY922), Alvespimycin (17-DMAG) HCl, Ganetespib (STA-9090), Onalespib (AT13387), Gleldananycin (NSC 122750), SNX-2112 (PF-04928473), PF-04929113 (SNX-5422), KW-2478, Cucurbitacin D, VER155008, VER-50589, CH5138303, VER-49009, NMS-E973, Zelavespib (PU-H71), HSP990 (NVP-HSP990), XL888 NVP-BEP800, 131113021 or a combination thereof. Such heat shock factor inhibitors can be obtained from Tocris.com.

As used herein, “solid tumor” is intended to include, but not be limited to, the following sarcomas and carcinomas: fibrosarcoma, myxosarcoma, liposarcoma, chondrosarcoma, osteogenic sarcona, chordoma, angiosarcorna, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminonma, embryonal carcinoma, Wilms' tumor, cervical cancer, testicular tumor, lung carcinoma, small cell lung carcinoma, bladder carcinoma, epithelial carcinoma, gliona, astrocytoma, medulloblastoma, craniopharyngioma, ependymoma, pinealoma, hemangioblastoma, acoustic neuroma, oligodendroglioma, meningioma, melanomna, neuroblastorna, and retinoblastoma. Solid tumor is also intended to encompass epithelial cancers.

### Zinc Finger Protein (ZNF92)

ZNF92 is a zinc finger protein that functions as transcription factor that binds nucleic acids and regulates transcription. The ZNF92 gene is located on chromosome 7 (Gene ID: 168374; location NC_000007.14 (65373855.65401 136), An example of an amino acid sequence for ZNF92 isoform 1 is available as UNIPROT accession no.

```
                10 20 30 40
MGPLTFRDVK IEFSLEEWQC LDTAQRNLYR DVMLENYRNL
                50 60 70 80
VFLGIAVSKP DLITWLEQGK EPWNLKRHEM VDKTPVMCSH
                90 100 110 120
FAQDVWPEHS IKDSFQKVIL RTYGKYGHEN LQLRKDHKSV
              130 140 150 160
DACKVYKGGY NGLNQCLTTT DSKIFQCDKY VKVFHKFPNV
              170 180 190 200
NRNKIRHTGK KPFKCKNRGK SFCMLSQLTQ HKKIHTREYS
              210 220 230 240
YKCEECGKAF NWSSTLTKHK IIHTGEKPYK CEECGKAFNR
              250 260 270 280
SSNLTKHKII HTGEKPYKCE ECGKAFNRSS TLTKHKRIHT
              290 300 310 320
EEKPYKCEEC GKAFNQFSIL NKHKRIHMED KPYKCEECGK
              330 340 350 360
AFRVFSILKK HKIIHTGEKP YKCEECGKAF NQFSNLTKHK
              370 380 390 400
IIHTGEKPYK CDECGKAFNQ SSTLTKHKRI HTGEKPYKCE
              410 420 430 440
ECGKAFKQSS TLTEHKIIHT GEKPYKCEKC GKAFSWSSAF
              450 460 470 480
TKHKRNHMED KPYKCEECGK AFSVFSTLTK HKIIHTREKP
              490 500 510 520
YKCEECGKAF NQSSIFTKHK IIHTEGKSYK CEKCGNAFNQ
              530 540 550 560
SSNLTARKII YTGEKPYKYE ECDKAFNKFS TLITHQIIYT
              570 580
GEKPCKHECG RAFNKSSNYT KEKLQT
```

A cDNA sequence encoding the SEQ ID NO:1 ZNF92 protein is available as NCBI accession no. BC040594.1, shown below as SEQ ID NO:2

| | |
|---|---|
| 1 | CTCTCGCTGC AGCCGGCGCT CCACGTCTAG TCTTCACTGC |
| 41 | TCTGCGTCCT GTGCTGATAA AGGCTCGCCG CTGTGACCCT |
| 81 | GTTACCTGCA AGAACTTGGA GGTTCACAGC TAAGACGCCA |
| 121 | GGACCCCCTG GAAGCCTAGA AATGGGACCA CTGACATTTA |
| 161 | GGGATGTGAA AATAGAATTC TCTCTAGAGG AATGGCAATG |
| 201 | CCTGGACACT GCGCAGCGGA ATTTATATAG AGATGTGATG |
| 241 | TTAGAGAACT ACAGAAACCT GGTCTTCCTT GGTATTGCTG |
| 281 | TCTCTAAGCC AGACCTGATC ACCTGGCTGG AGCAAGGAAA |
| 321 | AGAGCCCTGG AATCTGAAGA GACATGAGAT GGTAGACAAA |
| 361 | ACCCCAGTTA TGTGTTCTCA TTTTGCCCAA GATGTTTGGC |
| 401 | CAGAGCACAG CATAAAAGAT TCTTTCCAAA AAGTGATACT |
| 441 | GAGAACATAT GGAAAATATG GACATGAGAA TTTACAGCTA |
| 481 | AGAAAAGACC ATAAAAGTGT GGATGCATGT AAGGTGTACA |
| 521 | AAGGAGGTTA TAATGGACTT AACCAGTGTT TGACAACTAC |
| 561 | TGACAGCAAG ATATTTCAGT GTGATAAATA TGTGAAAGTC |
| 601 | TTTCATAAAT TTCCAAATGT AAATAGAAAT AAGATAAGAC |
| 641 | ATACTGGAAA GAAACCTTTC AAATGTAAAA ACCGTGGCAA |
| 681 | ATCATTTTGC ATGCTTTCAC AATTAACTCA ACATAAGAAA |
| 721 | ATTCATACTA GAGAGTATTC TTACAAATGT GAAGAATGTG |
| 761 | GTAAAGCCTT TAACTGGTCC TCAACCCTTA CTAAACATAA |
| 801 | GATAATTCAT ACTGGAGAAA AACCCTACAA ATGTGAAGAA |
| 841 | TGTGGCAAAG CTTTTAACCG GTCCTCAAAT CTTACTAAAC |
| 881 | ATAAAATAAT TCATACTGGA GAGAAACCCT ACAAATGTGA |
| 921 | AGAATGTGGC AAAGCTTTTA ACCGGTCCTC AACCCTTACT |
| 961 | AAACATAAAA GAATTCATAC AGAAGAGAAA CCCTACAAAT |
| 1001 | GTGAAGAATG TGGCAAGGCC TTTAACCAGT TCTCGATTCT |
| 1041 | TAATAAACAT AAGAGAATTC ATATGGAAGA TAAACCCTAC |
| 1081 | AAATGTGAAG AATGTGGCAA AGCCTTTAGA GTATTCTCAA |
| 1121 | TTCTTAAAAA ACATAAGATA ATCCATACTG GGGAAAAACC |
| 1161 | ATACAAATGT GAAGAATGTG GCAAAGCCTT TAACCAGTTC |
| 1201 | TCAAACCTTA CTAAACATAA GATAATTCAT ACTGGAGAGA |
| 1241 | AACCCTACAA ATGTGATGAA TGTGGCAAAG CCTTTAACCA |
| 1281 | GTCCTCAACC CTTACTAAAC ATAAAAGAAT TCATACGGGA |
| 1321 | GAAAAACCCT ACAAATGTGA AGAATGTGGC AAAGCTTTTA |
| 1361 | AACAGTCCTC AACCCTTACT GAACATAAGA TAATTCATAC |
| 1401 | TGGAGAGAAA CCCTACAAAT GTGAAAAATG TGGCAAGGCC |
| 1441 | TTTAGCTGGT CCTCAGCTTT TACTAAACAT AAGAGAAATC |
| 1481 | ATATGGAAGA TAAACCCTAC AAATGTGAAG AATGTGGCAA |
| 1521 | AGCCTTTAGT GTATTCTCAA CCCTTACTAA ACATAAAATA |
| 1561 | ATTCATACTA GAGAAAAACC CTACAAATGT GAAGAATGTG |
| 1601 | GCAAAGCCTT TAACCAGTCC TCAATTTTTA CTAAACATAA |
| 1641 | GATAATTCAC ACTGAAGGGA AATCCTACAA ATGTGAAAAA |
| 1681 | TGTGGCAATG CTTTTAACCA GTCCTCAAAC CTTACTGCAC |
| 1721 | GTAAGATAAT TTATACTGGA GAGAAACCCT ACAAATATGA |
| 1761 | AGAATGTGAC AAAGCCTTTA ACAAGTTCTC AACCCTTATT |
| 1801 | ACACATCAGA TAATTTATAC TGGAGAGAAA CCCTGCAAAC |
| 1841 | ATGAATGTGG CAGAGCCTTT AACAAATCCT CAAATTATAC |
| 1881 | TAAAGAGAAA CTACAAACCT GAAAGATGTG ACAATGATTT |
| 1921 | TCACTACACC TCAAACTTTT CTAAACATAA ACCATATTGG |
| 1961 | TGCCCTAGAA ATGTGAGGAA TATGACAAGG ACTTTAAATG |
| 2001 | GTTGTCACGC TTGATTGTAG GTAAGATAAT TTATATTGGA |
| 2041 | GAAAAATCCT CCAAGTATGA AGAATGTGGC AAACTTTTAA |
| 2081 | CCAATCCTCA CACCTTATTG CACAGGAAAG CATTTATACT |
| 2121 | TGAGAAAAAT TGTATAAAGA ATATGGAAAA GCCATTTATA |
| 2161 | TCTGCTCACA TGTAAAAACA TCAGTTCATA CTTAATAAAA |
| 2201 | TGCAATTACC GTCAAATCTT TCAGAAAATA TAAGCCTTTA |
| 2241 | ATACGAGGAA GAGTATTCTT AAGATGAACA TTACAAATAG |
| 2281 | AAAGAGGGTT GTAGTACCTT TAGTTTTATG ATAGATCTTA |
| 2321 | TTGTACACAT TTTGTACCAG AGGAAAACCC TAAAGCATTA |
| 2361 | GTTGCTCAAA CTTTGTTCGA CATCAGGGAA TTTGTATTGG |
| 2401 | AGAAAAACCC TGCAAATGTA ATAAATATGG AAAAACATTT |
| 2441 | TTTCAAAAAC TACAGCTTGG AAAACATCAG AGAGTTCATA |
| 2481 | CTAAAATATA TTTTTGCAGA TGCAGTAAAT ATGAAAAATA |
| 2521 | TTTAATCCCA AATTAAGTCT ATGTAAATAT CAGAATTCAC |
| 2561 | AGTAGAAATC ATAAGGCATA AGGCACTGAT ACTTCAGACA |
| 2601 | TTACACTAAA TTAGAGTGTT GAGTATAGGA GATCCAAAAC |
| 2641 | TAAAATTGTT AGGTAAGTTA TTTATATATA ACTTTAAAAG |
| 2681 | AAGTAGAAGA TTTTTTGGAG ATTTATAATT ACATTCAAAG |
| 2721 | TATACTTTTT TCTTGAAAAA AATTACAGAT TTTTTGAAAA |
| 2761 | GCAATTGATG TAATTTAACT CTCAAATTCA TGTTTTTCTT |
| 2801 | CATTCCTATT ATATTCACAT GTGAAAGCAA GTGATCTGTT |
| 2841 | GTTGCTGAAT CAGAGATATG AGAGATTCTT TTTTATAGGT |
| 2881 | GGGCATTATT TATGCCCCTT TCTGTGGAAG AGTAAGAAAA |
| 2921 | TTAAAATACA AGATGCATGA GGAAAATGTA GAGATGCTCT |
| 2961 | TTGTGATTAA CTTAGAATAT TAAGTGCTAC TTGACGTACA |
| 3001 | TGTTCAGACT AACATTCTTT TGCAGTATAG TGAGAAAAAA |
| 3041 | ACATTTTAAA ATTAATTATC ATTTTGTTGA TTGTGCTTTT |
| 3081 | ATGTAATAAA ATGCAGTACT TTAAAACAAA AAAAAAAAAA |
| 3121 | AAA |

The ET-9 signature genes are listed below in Table 1 with UNIPROT accession numbers and examples of amino acid sequences.

TABLE 1
ET-9 signature genes

| Entrez ID | ET-9 Name & Example of Human Amino Acid Sequence |
|---|---|
| 9289 | GPRS6 (Adhesion G protein-coupled receptor G1; |
| Uniprot | SEQ ID NO: 3) |
| Q9Y653 | 10 20 30 40 50 |
| NCBI mRNA | MTPQSLLQTT LFLLSLLFLV QGAHGRGHRE DERFCSQRNQ THRSSLHYKP |
| AY358400.1 | 60 70 80 90 100 |
| | TPDLRISIEN SEEALTVHAP FPAAHPASRS FPDPRGLYHF CLYWNRHAGR |
| | 110 120 130 140 150 |
| | LHLLYGKRDF LLSDKASSLL CFQHQEESLA QGPPLLATSV TSWWSPQNIS |
| | 160 170 180 190 200 |
| | LPSAASFTFS FHSPPHTAAH NASVDMCELK RDLQLLSQFL KHPQKASRRP |
| | 210 220 230 240 250 |
| | SAAPASQQLQ SLESKLTSVR FMGDMVSFEE DRINATVWKL QPTAGLQDLH |
| | 260 270 280 290 300 |
| | IHSRQEEEQS EIMEYSVLLP RTLFQRTKGR SGEAEKRLLL VDFSSQALFQ |
| | 310 320 330 340 350 |
| | DKNSSQVLGE KVLGIVVQNT KVANLTEPVV LTFQHQLQPK NVTLQCVFWV |
| | 360 370 380 390 400 |
| | EDPTLSSPGH WSSAGCETVR RETQTSCFCN HLTYFAVLMV SSVEVDAVHK |
| | 410 420 430 440 450 |
| | HYLSLLSYVG CVVSALACLV TIAAYLCSRV PLPCRRKPRD YTIKVHMNLL |
| | 460 470 480 490 500 |
| | LAVFLLDTSF LLSEPVALTG SEAGCRASAI FLHFSLLTCL SWMGLEGYNL |
| | 510 520 530 540 550 |
| | YRLVVEVFGT YVPGYLLKLS AMGWGFPIFL VTLVALVDVD NYGPIILAVH |
| | 560 570 580 590 600 |
| | RTPEGVIYPS MCWIRDSLVS YITNLGLFSL VFLFNMAMLA TMVVQILRLR |
| | 610 620 630 640 650 |
| | PHTQKWSHVL TLLGLSLVLG LPWALIFFSF ASGTFQLVVL YLFSIITSFQ |
| | 660 670 680 690 |
| | GFLIFIWYWS MRLQARGGPS PLKSNSDSAR LPISSGSTSS SRI |
| 84929 | FIBCD1 (Fibrinogen C domain containing 1; |
| Uniprot | SEQ ID NO: 4) |
| Q8N539 | 10 20 30 40 50 |
| NCBI mRNA | MVNDRWKTMG GAAQLEDRPR DKPQRPSCGY VLCTVLLALA VLLAVAVTGA |
| BC032953 | 60 70 80 90 100 |
| | VLFLNHAHAP GTAPPPVVST GAASANSALV TVERADSSHL SILIDPRCPD |
| | 110 120 130 140 150 |
| | LTDSFARLES AQASVLQALT EHQAQPRLVG DQEQELLDTL ADQLPRLLAR |
| | 160 170 180 190 200 |
| | ASELQTECMG LRKGHGTIGQ GLSALQSEQG RLIQLLSESQ GHMAHLVNSV |
| | 210 220 230 240 250 |
| | SDILDALQRD RGLGRPRNKA DLQRAPARGT RPRGCATGSR PRDCLDVLLS |
| | 260 270 280 290 300 |
| | GQQDDGVYSV FPTHYPAGFQ VYCDMRTDGG GWTVFQRRED GSVNFFRGWD |
| | 310 320 330 340 350 |
| | AYRDGFGRLT GEHWLGLKRI HALTTQAAYE LHVDLEDFEN GTAYARYGSF |
| | 360 370 380 390 400 |
| | GVGLFSVDPE EDGYPLTVAD YSGTAGDSLL KHSGMRFTTK DRDSDHSENN |
| | 410 420 430 440 450 |
| | CAAFYRGAWW YRNCHTSNLN GQYLRGAHAS YADGVEWSSW TGWQYSLKFS |
| | 460 |
| | EMKIRPVRED R |
| 81544 | GDPD5 (Glycerophosphodiester phosphodiesterase domain |
| Uniprot | containing 5; SEQ ID NO: 5) |
| Q8WTR4 | 10 20 30 40 50 |
| NCBI | MVRHQPLQYY EPQLCLSCLT GIYGCRWKRY QRSHDDTTPW ERLWFLLLTF |
| mRNA | 60 70 80 90 100 |
| NM_ | TFGLTLTWLY FWWEVENDYD EFNWYLYNRM GYWSDWPVPI LVTTAAAFAY |
| 030792.8 | 110 120 130 140 150 |
| | IAGLLVLALC HIAVGQQMNL HWLHKIGLVV ILASTVVAMS AVAQLWEDEW |
| | 160 170 180 190 200 |
| | EVLLISLQGT APFLHVGAVA AVTMLSWIVA GQFARAERTS SQVTILCTFF |
| | 210 220 230 240 250 |
| | TVVFALYLAP LTISSPCIME KKDLGPKPAL IGHRGAPMLA PEHTLMSFRK |
| | 260 270 280 290 300 |
| | ALEQKLYGLQ ADITISLDGV PFLMHDTTLR RTTNVEEEFP ELARRPASML |
| | 310 320 330 340 350 |
| | NWTTLQRLNA GQWFLKTDPF WTASSLSPSD HREAQNQSIC SLAELLELAK |
| | 360 370 380 390 400 |
| | GNATLLLNLR DPPREHPYRS SFINVTLEAV LHSGFPQHQV MWLPSRQRPL |
| | 410 420 430 440 450 |
| | VRKVAPGFQQ TSGSKEAVAS LRRGHIQRLN LRYTQVSRQE LRDYASWNLS |
| | 460 470 480 490 500 |
| | VNLYTVNAPW LFSLLWCAGV PSVTSDNSHA LSQVPSPLWI MPPDEYCLMW |
| | 510 520 530 540 550 |
| | VTADLVSFTL IVGIFVLQKW RIGGIRSYNP EQIMLSAAVR RTSRDVSIMK |
| | 560 570 580 590 600 |
| | EKLIFSEISD GVEVSDVLSV CSDNSYDTYA NSTATPVGPR GGGSHTKTLI |
| | ERSGR |
| 56241 | SUSD2 (Sushi domain containing 2; SEQ ID NO: 6) |
| Uniprot | 10 20 30 40 50 |
| Q9UGT4 | MKPALLPWAL LLLATALGPG PGPTADAQES CSMRCGALDG PCSCHPTCSG |
| NCBI mRNA | 60 70 80 90 100 |
| BC033107.1 | LGTCCLDFRD FCLEILPYSG SMMGGKDFVV RHFKMSSPTD ASVICRFKDS |
| | 110 120 130 140 150 |
| | IQTLGHVDSS GQVHCVSPLL YESGRIPFTV SLDNGHSFPR AGTWLAVHPN |
| | 160 170 180 190 200 |
| | KVSMMEKSEL VNETRWQYYG TANTSGNLSL TWHVKSLPTQ TITIELWGYE |
| | 210 220 230 240 250 |
| | ETGMPYSQEW TAKWSYLYPL ATHIPNSGSF TFTPKPAPPS YQRWRVGALR |
| | 260 270 280 290 300 |
| | IIDSKNYAGQ KDVQALWTND HALAWHLSDD FREDPVAWAR TQCQAWEELE |
| | 310 320 330 340 350 |
| | DQLPNFLEEL PDCPCTLTQA RADSGRFFTD YGCDMEQGSV CTYHPGAVHC |
| | 360 370 380 390 400 |
| | VRSVQASLRY GSGQQCCYTA DGTQLLTADS SGGSTPDRGH DWGAPPFRTP |
| | 410 420 430 440 450 |
| | PRVPSMSHWL YDVLSFYYCC LWAPDCPRYM QRRPSNDCRN YRPPRLASAF |
| | 460 470 480 490 500 |
| | GDPHFVTFDG TNFTFNGRGE YVLLEAALTD LRVQARAQPG TMSNGTETRG |
| | 510 520 530 540 550 |
| | TGLTAVAVQE GNSDVVEVRL ANRTGGLEVL LNQEVLSFTE QSWMDLKGMF |
| | 560 570 580 590 600 |
| | LSVAAGDRVS IMLASGAGLE VSVQGPFLSV SVLLPEKFLT HTHGLIGTLN |
| | 610 620 630 640 650 |
| | NDPTDDFTLH SGRVIPPGTS PQELFLFGAN WTVHNASSLL TYDSWFLVHN |
| | 660 670 680 690 700 |
| | FLYQPKHDPT FEPLFPSETT LNPSLAQEAA KLCGDDHFCN FDVAATGSLS |
| | 710 720 730 740 750 |
| | TGTATRVAHQ LHQRRMQSLQ PVVSCGWLAP PPNGQKEGNR YLAGSTIYFH |
| | 760 770 780 790 800 |
| | CDNGYSLAGA ETSTCQADGT WSSPTPKCQP GRSYAVLLGI IFGGLAVVAA |
| | 810 820 |
| | VALVYVLLRR RKGNTHVWGA QP |
| 27092 | CACNG4 (Calcium voltage-gated channel auxiliary subunit |
| Uniprot | gamma 4; SEQ ID NO: 7) |
| Q9UBN1 | 10 20 30 40 50 |
| NCBI mRNA | MVRCDRGLQM LLTTAGAFAA FSLMAIAIGT DYWLYSSAHI CNGTNLTMDD |
| AF162692.1 | 60 70 80 90 100 |
| | GPPPRRARGD LTHSGLWRVC CIEGIYKGHC FRINHFPEDN DYDHDSSEYL |
| | 110 120 130 140 150 |
| | LRIVRASSVF PILSTILLLL GGLCIGAGRI YSRKNNIVLS AGILFVAAGL |
| | 160 170 180 190 200 |
| | SNIIGIIVYI SSNTGDPSDK RDEDKKNHYN YGWSFYFGAL SFIVAETVGV |
| | 210 220 230 240 250 |
| | LAVNIYIEKN KELRFKTKRE FLKASSSSPY ARMPSYRYRR RRSRSSSRST |
| | 260 270 280 290 300 |
| | EASPSRDVSP MGLKITGAIP MGELSMYTLS REPLKVTTAA SYSPDQEASF |
| | 310 320 |
| | LQVHDFFQQD LKEGFHVSML NRRTTPV |
| 6376 | CX3CL1 (C-X3-C motif chemokine ligand 1; SEQ ID NO: 8) |
| Uniprot | 10 20 30 40 50 |
| P78423 | MAPISLSWLL RLATFCHLTV LLAGQHHGVT KCNITCSKMT SKIPVALLIH |
| NCBI mRNA | 60 70 80 90 100 |
| BC001163.1 | YQQNQASCGK RAIILETRQH RLFCADPKEQ WVKDAMQHLD RQAAALTRNG |
| | 110 120 130 140 150 |
| | GTFEKQIGEV KPRTTPAAGG MDESVVLEPE ATGESSSLEP TPSSQEAQRA |
| | 160 170 180 190 200 |
| | LGTSPELPTG VTGSSGTRLP PTPKAQDGGP VGTELFRVPP VSTAATWQSS |
| | 210 220 230 240 250 |
| | APHQPGPSLW AEAKTSEAPS TQDPSTQAST ASSPAPEENA PSEGQRVWGQ |
| | 260 270 280 290 300 |
| | GQSPRPENSL EREEMGPVPA HTDAFQDWGP GSMAHVSVVP VSSEGTPSRE |
| | 310 320 330 340 350 |
| | PVASGSWTPK AEEPIHATMD PQRLGVLITP VPDAQAATRR QAVGLLAFLG |
| | 360 370 380 390 |
| | LLFCLGVAMF TYQSLQGCPR KMAGEMAEGL RYIPRSCGSN SYVLVPV |
| 3488 | IGFBP5 (insulin like growth factor binding protein 5; |
| Uniprot | SEQ ID NO: 9) |
| P24593 | 10 20 30 40 50 |
| NCBI mRNA | MVLLTAVLLL LAAYAGPAQS LGSFVHCEPC DEKALSMCPP SPLGCELVKE |
| AF055033.1 | 60 70 80 90 100 |
| | PGCGCCMTCA LAEGQSCGVY TERCAQGLRC LPRQDEEKPL HALLHGRGVC |
| | 110 120 130 140 150 |
| | LNEKSYREQV KIERDSREHE EPTTSEMAEE TYSPKIFRPK HTRISELKAE |
| | 160 170 180 190 200 |
| | AVKKDRRKKL TQSKFVGGAE NTAHPRIISA PEMRQESEQG PCRRHMEASL |
| | 210 220 230 240 250 |
| | QELKASPRMV PRAVYLPNCD RKGFYKRKQC KPSRGRKRGI CWCVDKYGMK |
| | 260 270 |
| | LPGMEYVDGD FQCHTFDSSN VE |
| 4135 | MAP6 (microtubule associated protein 6; SEQ ID NO: 10) |
| Uniprot | 10 20 30 40 50 |
| Q96JE9 | MAWPCITRAC CIARFWNQLD KADIAVPLVF TKYSEATEHP GAPPQPPPPQ |
| NCBI mRNA | 60 70 80 90 100 |
| BC139780.1 | QQAQPALAPP SARAVAIETQ PAQGELDAVA RATGPAPGPT GEREPAAGPG |
| | 110 120 130 140 150 |
| | RSGPGPGLGS GSTSGPADSV MRQDYRAWKV QRPEPSCRPR SEYQPSDAPF |
| | 160 170 180 190 200 |
| | ERETQYQKDF RAWPLPRRGD HPWIPKPVQI SAASQASAPI LGAPKRRPQS |
| | 210 220 230 240 250 |
| | QERWPVQAAA EAREQEAAPG GAGGLAAGKA SGADERDTRR KAGPAWIVRR |
| | 260 270 280 290 300 |
| | AEGLGHEQTP LPAAQAQVQA TGPEAGRGRA AADALNRQIR EEVASAVSSS |
| | 310 320 330 340 350 |
| | YRNEFRAWTD IKPVKPIKAK PQYKPPDDKM VHETSYSAQF KGEASKPTTA |
| | 360 370 380 390 400 |
| | DNKVIDRRRI RSLYSEPFKE PPKVEKPSVQ SSKPKKTSAS HKPTRKAKDK |
| | 410 420 430 440 450 |
| | QAVSGQAAKK KSAEGPSTTK PDDKEQSKEM NNKLAEAKES LAQPVSDSSK |
| | 460 470 480 490 500 |
| | TQGPVATEPD KDQGSVVPGL LKGQGPMVQE PLKKQGSVVP GPPKDLGPMI |
| | 510 520 530 540 550 |
| | PLPVKDQDHT VPEPLKNESP VISAPVKDQG PSVPVPPKNQ SPMVPAKVKD |
| | 560 570 580 590 600 |
| | QGSVVPESLK DQGPRIPEPV KNQAPMVPAP VKDEGPMVSA SVKDQGPMVS |
| | 610 620 630 640 650 |
| | APVKDQGPIV PAPVKGEGPI VPAPVKDEGP MVSAPIKDQD PMVPEHPKDE |
| | 660 670 680 690 700 |
| | SAMATAPIKN QGSMVSEPVK NQGLVVSGPV KDQDVVVPEH AKVHDSAVVA |
| | 710 720 730 740 750 |
| | PVKNQGPVVP ESVKNQDPIL PVLVKDQGPT VLQPPKNQGR IVPEPLKNQV |
| | 760 770 780 790 800 |
| | PIVPVPLKDQ DPLVPVPAKD QGPAVPEPLK TQGPRDPQLP TVSPLPRVMI |
| | 810 |
| | PTAPHTEYIE SSP |
| 26112 | CCDC69 coiled-coil domain containing 69 (SEQ ID NO: 11) |
| Uniprot | 10 20 30 40 50 |
| A6NI79 | MGCRHSRLSS CKPPKKKRQE PEPEQPPRPE PHELGPLNGD TAITVQLCAS |
| NCBI mRNA | 60 70 80 90 100 |
| NM_015621.3 | EEAERHQKDI TRILQQHEEE KKKWAQQVEK ERELELRDRL DEQQRVLEGK |
| | 110 120 130 140 150 |
| | NEEALQVERA SYEQEKEALT HSFREASSTQ QETIDRLTSQ LEAFQAKMKR |
| | 160 170 180 190 200 |
| | VEESILSRNY KKHIQDYGSP SQFWEQELES LHFVIEMKNE RIHELDRRLI |
| | 210 220 230 240 250 |
| | LMETVKEKNL ILEEKITTLQ QENEDLHVRS RNQVVLSRQL SEDLLLTREA |
| | 260 270 280 290 |
| | LEKEVQLRRQ LQQEKEELLY RVLGANASPA FPLAPVTPTE VSFLAT |

TABLE 2
ET-60 signature genes

| ID | ET-60 Name & Example of Human Amino Acid Sequence |
|---|---|
| ABTB1 | Ankyrin repeat and BTB/POZ domain-containing protein 1 (SEQ ID NO: 12) |
| Uniprot | 10 20 30 40 50 |
| Q969K4 | MDTSDLFASC RKGDVGRVRY LLEQRDVEVN VRDKWDSTPL YYACLCGHEE |
| NCBI mRNA | 60 70 80 90 100 |
| NM_032548.4 | LVLYLLANGA RCEANTFDGE RCLYGALSDP IRRALRDYKQ VTASCRRRDY |
| | 110 120 130 140 150 |
| | YDDFLQRLLE QGIHSDVVFV VHGKPFRVHR CVLGARSAYF ANMLDTKWKG |
| | 160 170 180 190 200 |
| | KSVVVLRHPL INPVAFGALL QYLYTGRLDI GVEHVSDCER LAKQCQLWDL |
| | 210 220 230 240 250 |
| | LSDLEAKCEK VSEFVASKPG TCVKVITIEP PPADPRLRED MALLADCALP |
| | 260 270 280 290 300 |
| | PELRGDLWEL PFPCPDGFNS CPDICFRVAG CSFLCHKAFF CGRSDYFRAL |
| | 310 320 330 340 350 |
| | LDDHFRESEE PATSGGPPAV TLHGISPDVE THVLYYMYSD HTELSPEAAY |
| | 360 370 380 390 400 |
| | DVLSVADMYL LPGLKRLCGR SLAQMLDEDT VVGVWRVAKL FRLARLEDQC |
| | 410 420 430 440 450 |
| | TEYMAKVIEK LVEREDEVEA VKEEAAAVAA ROETDSIPLV DDIRFHVAST |
| | 460 470 |
| | VQTYSAIEEA QQRLRALEDL LVSIGLDC |
| BCAS4 | Breast carcinoma-amplified sequence 4 (SEQ ID NO :13) |
| Uniprot | 10 20 30 40 50 |
| Q8TDM0 | MQRTGGGAPR PGRNHGLPGS LRQPDPVALL MLLVDADQPE PMRSGARELA |
| | 60 70 80 90 100 |
| | LFLTPEPGAE AKEVEETIEG MLLRLEEFCS LADLIRSDTS QILEENIPVL |
| | 110 120 130 140 150 |
| | KAKLTEMRGI YAKVDRLEAF VKMVGHHVAF LEADVLQAER DHGAFPQALR |
| | 160 170 180 190 200 |
| | RWLGSAGLPS FRNVECSGTI PARCNLRLPG SSDSPASASQ VAGITEVTCT |
| | 210 |
| | GARDVRAAHT V |
| BNIPL | Bcl-2/adenovirus E1B 19 kDa-interacting protein 2-like protein (SEQ |
| Uniprot | ID NO: 14) |
| Q7Z465 | 10 20 30 40 50 |
| | MGTIQEAGKK TDVGVREIAE APELGAALRH GELELKEEWQ DEEFPRLLPE |
| | 60 70 80 90 100 |
| | EAGTSEDPED PKGDSQAAAG TPSTLALCGQ RPMRKRLSAP ELRLSLTKGP |
| | 110 120 130 140 150 |
| | GNDGASPTQS APSSPDGSSD LEIDELETPS DSEQLDSGHE FEWEDELPRA |
| | 160 170 180 190 200 |
| | EGLGTSETAE RLGRGCMWDV TGEDGHHWRV FRMGPREQRV DMTVIEPYKK |
| | 210 220 230 240 250 |
| | VLSHGGYHGD GLNAVILFAS CYLPRSSIPN YTYVMEHLER YMVGTLELLV |
| | 260 270 280 290 300 |
| | AENYLLVHLS GGTSRAQVPP LSWIRQCYRT LDRRLRKNLR ALVVVHATWY |
| | 310 320 330 340 350 |
| | VKAFLALLRP FISSKFTRKI RFLDSLGELA QLISLDQVHI PEAVRQLDRD |
| | LHGSGGT |
| BOC | Brother of CDO (SEQ ID NO: 15) |
| Uniprot | 10 20 30 40 50 |
| Q9BWV1 | MLRGTMTAWR GMRPEVTLAC LLLATAGCFA DLNEVPQVTV QPASTVQKPG |
| | 60 70 80 90 100 |
| | GTVILGCVVE PPRMNVTWRL NGKELNGSDD ALGVLITHGT LVITALNNHT |
| | 110 120 130 140 150 |
| | VGRYQCVARM PAGAVASVPA TVTLANLQDF KLDVQHVIEV DEGNTAVIAC |
| | 160 170 180 190 200 |
| | HLPESHPKAQ VRYSVKQEWL EASRGNYLIM PSGNLQIVNA SQEDEGMYKC |
| | 210 220 230 240 250 |
| | AAYNPVTQEV KTSGSSDRLR VRRSTAEAAR IIYPPEAQTI IVTKGQSLIL |
| | 260 270 280 290 300 |
| | ECVASGIPPP RVTWAKDGSS VTGYNKTRFL LSNLLIDTTS EEDSGTYRCM |
| | 310 320 330 340 350 |
| | ADNGVGQPGA AVILYNVQVF EPPEVTMELS QLVIPWGQSA KLTCEVRGNP |
| | 360 370 380 390 400 |
| | PPSVLWLRNA VPLISSQRLR LSRRALRVLS MGPEDEGVYQ CMAENEVGSA |
| | 410 420 430 440 450 |
| | HAVVQLRTSR PSITPRLWQD AELATGTPPV SPSKLGNPEQ MLRGQPALPR |
| | 460 470 480 490 500 |
| | PPTSVGPASP QCPGEKGQGA PAEAPIILSS PRTSKTDSYE LVWRPRHEGS |
| | 510 520 530 540 550 |
| | GRAPILYYVV KHRKVTNSSD DWTISGIPAN QHRLTLTRLD PGSLYEVEMA |
| | 560 570 580 590 600 |
| | AYNCAGEGQT AMVTFRTGRR PKPEIMASKE QQIQRDDPGA SPQSSSQPDH |
| | 610 620 630 640 650 |
| | GRISPPEAPD RPTISTASET SVYVTWIPRG NGGFPIQSFR VEYKKLKKVG |
| | 660 670 680 690 700 |
| | DWILATSAIP PSRLSVEITG LEKGTSYKFR VRALNMLGES EPSAPSRPYV |
| | 710 720 730 740 750 |
| | VSGYSGRVYE RPVAGPYITF TDAVNETTIM LKWMYIPASN NNTPIHGFYI |
| | 760 770 780 790 800 |
| | YYRPTDSDND SDYKKDMVEG DKYWHSISHL QPETSYDIKM QCFNEGGESE |
| | 810 820 830 840 850 |
| | FSNVMICETK ARKSSGQPGR LPPPTLAPPQ PPLPETIERP VGTGAMVARS |
| | 860 870 880 890 900 |
| | SDLPYLIVGV VLGSIVLIIV TFIPFCLWRA WSKQKHTTDL GFPRSALPPS |
| | 910 920 930 940 950 |
| | CPYTMVPLGG LPGHQASGQP YLSGISGRAC ANGIHMNRGC PSAAVGYPGM |
| | 960 970 980 990 1000 |
| | KPQQHCPGEL QQQSDTSSLL RQTHLGNGYD PQSHQITRGP KSSPDEGSFL |
| | 1010 1020 1030 1040 1050 |
| | YTLPDDSTHQ LLQPHHDCCQ RQEQPAAVGQ SGVRRAPDSP VLEAVWDPPF |
| | 1060 1070 1080 1090 1100 |
| | HSGPPCCLGL VPVEEVDSPD SCQVSGGDWC POHPVGAYVG QEPGMQLSPG |
| | 1110 |
| | PLVRVSFETP PLTI |
| CACNG4 | Voltage-dependent calcium channel gamma-4 subunit (SEQ ID NO: 16) |
| Uniprot | 10 20 30 40 50 |
| Q9UBN1 | MVRCDRGLQM LLTTAGAFAA FSLMAIAIGT DYWLYSSAHI CNGTNLTMDD |
| | 60 70 80 90 100 |
| | GPPPRRARGD LTHSGLWRVC CIEGIYKGHC FRINHFPEDN DYDHDSSEYL |
| | 110 120 130 140 150 |
| | LRIVRASSVF PILSTILLLL GGLCIGAGRI YSRKNNIVLS AGILFVAAGL |
| | 160 170 180 190 200 |
| | SNIIGIIVYI SSNTGDPSDK RDEDKKNHYN YGWSFYFGAL SFIVAETVGV |
| | 210 220 230 240 250 |
| | LAVNIYIEKN KELRFKTKRE FLKASSSSPY ARMPSYRYRR RRSRSSSRST |
| | 260 270 280 290 300 |
| | EASPSRDVSP MGLKITGAIP MGELSMYTLS REPLKVTTAA SYSPDQEASE |
| | 310 320 |
| | LQVHDFFQQD LKEGFHVSML NRRTTPV |
| CCDC69 | Voltage-dependent calcium channel gamma-4 subunit (SEQ ID NO: 17) |
| Uniprot | 10 20 30 40 50 |
| Q9UBN1 | MVRCDRGLQM LLTTAGAFAA FSLMAIAIGT DYWLYSSAHI CNGTNLTMDD |
| | 60 70 80 90 100 |
| | GPPPRRARGD LTHSGLWRVC CIEGIYKGHC FRINHFPEDN DYDHDSSEYL |
| | 110 120 130 140 150 |
| | LRIVRASSVF PILSTILLLL GGLCIGAGRI YSRKNNIVLS AGILFVAAGL |
| | 160 170 180 190 200 |
| | SNIIGIIVYI SSNTGDPSDK RDEDKKNHYN YGWSFYFGAL SFIVAETVGV |
| | 210 220 230 240 250 |
| | LAVNIYIEKN KELRFKTKRE FLKASSSSPY ARMPSYRYRR RRSRSSSRST |
| | 260 270 280 290 300 |
| | EASPSRDVSP MGLKITGAIP MGELSMYTLS REPLKVTTAA SYSPDQEASF |
| | 310 320 |
| | LQVHDFFQQD LKEGFHVSML NRRTTPV |
| CCND2 | G1/S-specific cyclin-D2 (SEQ ID NO: 18) |
| Uniprot | 10 20 30 40 50 |
| P30279 | MELLCHEVDP VRRAVRDRNL LRDDRVLQNL LTIEERYLPQ CSYFKCVQKD |
| | 60 70 80 90 100 |
| | IQPYMRRMVA TWMLEVCEEQ KCEEEVFPLA MNYLDRFLAG VPTPKSHLQL |
| | 110 120 130 140 150 |
| | LGAVCMFLAS KLKETSPLTA EKLCIYTDNS IKPQELLEWE LVVLGKLKWN |
| | 160 170 180 190 200 |
| | LAAVTPHDFI EHILRKLPQQ REKLSLIRKH AQTFIALCAT DFKFAMYPPS |
| | 210 220 230 240 250 |
| | MIATGSVGAA ICGLQQDEEV SSLTCDALTE LLAKITNTDV DCLKACQEQI |
| | 260 270 280 |
| | EAVLLNSLQQ YRQDQRDGSK SEDELDQAST PTDVRDIDL |
| CPA4 | Carboxypeptidase A4 (SEQ ID NO: 19) |
| Uniprot | 10 20 30 40 50 |
| Q9UI42 | MRWILFIGAL IGSSICGQEK FFGDQVLRIN VRNGDEISKL SQLVNSNNLK |
| | 60 70 80 90 100 |
| | LNFWKSPSSF NRPVDVLVPS VSLQAFKSFL RSQGLEYAVT IEDLQALLDN |
| | 110 120 130 140 150 |
| | EDDEMQHNEG QERSSNNFNY GAYHSLEAIY HEMDNIAADF PDLARRVKIG |
| | 160 170 180 190 200 |
| | HSFENRPMYV LKFSTGKGVR RPAVWLNAGI HSREWISQAT AIWTARKIVS |
| | 210 220 230 240 250 |
| | DYQRDPAITS ILEKMDIFLL PVANPDGYVY TQTQNRLWRK TRSRNPGSSC |
| | 260 270 280 290 300 |
| | IGADPNRNWN ASFAGKGASD NPCSEVYHGP HANSEVEVKS VVDFIQKHGN |
| | 310 320 330 340 350 |
| | FKGFIDLHSY SQLLMYPYGY SVKKAPDAEE LDKVARLAAK ALASVSGTEY |
| | 360 370 380 390 400 |
| | QVGPTCTTVY PASGSSIDWA YDNGIKFAFT FELRDTGTYG FLLPANQIIP |
| | 410 420 |
| | TAEETWLGLK TIMEHVRDNL Y |
| CROCC | Rootletin (SEQ ID NO: 20) |
| Uniprot | 10 20 30 40 50 |
| Q5TZA2 | MSLGLARAQE VELTLETVIQ TLESSVLCQE KGLGARDLAQ DAQITSLPAL |
| | 60 70 80 90 100 |
| | IREIVTRNLS QPESPVLLPA TEMASLLSLQ EENQLLQQEL SRVEDLLAQS |
| | 110 120 130 140 150 |
| | RAERDELAIK YNAVSERLEQ ALRLEPGELE TQEPRGLVRQ SVELRRQLQE |
| | 160 170 180 190 200 |
| | EQASYRRKLQ AYQEGQQRQA QLVQRLQGKI LQYKKRCSEL EQQLLERSGE |
| | 210 220 230 240 250 |
| | LEQQRLRDTE HSQDLESALI RLEEEQQRSA SLAQVNAMLR EQLDQAGSAN |
| | 260 270 280 290 300 |
| | QALSEDIRKV TNDWTRCRKE LEHREAAWRR EEESFNAYFS NEHSRLLLLW |
| | 310 320 330 340 350 |
| | RQVVGFRRLV SEVKMFTERD LLQLGGELAR TSRAVQEAGL GLSTGLRLAE |
| | 360 370 380 390 400 |
| | SRAEAALEKQ ALLQAQLEEQ LRDKVIREKD LAQQQMQSDL DKADLSARVT |
| | 410 420 430 440 450 |
| | ELGLAVKRLE KQNLEKDQVN KDLTEKLEAL ESLRLQEQAA LETEDGEGLQ |
| | 460 470 480 490 500 |
| | QTLRDLAQAV LSDSESGVQL SGSERTADAS NGSLRGISGQ RTPSPPRRSS |
| | 510 520 530 540 550 |
| | PGRGRSPRRG PSPACSDSST LALIHSALHK RQLQVQDMRG RYEASQDLLG |
| | 560 570 580 590 600 |
| | TERKQLSDSE SERRALEEQL QRLRDKTDGA MQAHEDAQRE VQRLRSANEL |
| | 610 620 630 640 650 |
| | LSREKSNLAH SLQVAQQQAE ELRQEREKLQ AAQEELRRQR DRLEEEQEDA |
| | 660 670 680 690 700 |
| | VQDGARVRRE LERSHRQLEQ LEGKRSVLAK ELVEVREALS RATLQRDMLQ |
| | 710 720 730 740 750 |
| | AEKAEVAEAL TKAEAGRVEL EISMTKLRAE EASLQDSLSK LSALNESLAQ |
| | 760 770 780 790 800 |
| | DKLDLNRLVA QLEEEKSALQ GRQRQAEQEA TVAREEQERL EELRLEQEVA |
| | 810 820 830 840 850 |
| | RQGLEGSERV AEQAQEALEQ QLPTLRHERS QLQEQLAQLS RQLSGREQEL |
| | 860 870 880 890 900 |
| | EQARREAQRQ VEALERAARE KEALAKEHAG LAVQLVAAER EGRTLSEEAT |
| | 910 920 930 940 950 |
| | RLRLEKEALE GSLFEVQRQL AQLEARREQL EAEGQALLLA KETLTGELAG |
| | 960 970 980 990 1000 |
| | LRQQIIATQE KASLDKELMA QKLVQAEREA QASLREQRAA HEEDLQRLQR |
| | 1010 1020 1030 1040 1050 |
| | EKEAAWRELE AERAQLQSQL QREQEELLAR LEAEKEELSE EIAALQQERD |
| | 1060 1070 1080 1090 1100 |
| | EGLLLAESEK QQALSLKESE KTALSEKLMG TRHSLATISL EMERQKRDAQ |
| | 1110 1120 1130 1140 1150 |
| | SRQEQDRSTV NALTSELRDL RAQREEAAAA HAQEVRRLQE QARDLGKQRD |
| | 1160 1170 1180 1190 1200 |
| | SCLREAEELR TQLRLLEDAR DGLRRELLEA QRKLRESQEG REVQRQEAGE |
| | 1210 1220 1230 1240 1250 |
| | LRRSLGEGAK EREALRRSNE ELRSAVKKAE SERISIKLAN EDKEQKLALL |
| | 1260 1270 1280 1290 1300 |
| | EEARTAVGKE AGELRTGLQE VERSRLEARR ELQELRRQMK MLDSENTRLG |
| | 1310 1320 1330 1340 1350 |
| | RELAELQGRL ALGERAEKES RRETLGLRQR LLKGEASLEV MRQELQVAQR |
| | 1360 1370 1380 1390 1400 |
| | KLQEQEGEFR TRERRLLGSL EEARGTEKQQ LDHARGLELK LEAARAEAAE |
| | 1410 1420 1430 1440 1450 |
| | LGLRLSAAEG RAQGLEAELA RVEVQRRAAE AQLGGLRSAL RRGLGLGRAP |
| | 1460 1470 1480 1490 1500 |
| | SPAPRPVPGS PARDAPAEGS GEGLNSPSTL ECSPGSQPPS PGPATSPASP |
| | 1510 1520 1530 1540 1550 |
| | DLDPEAVRGA LREFLQELRS AQRERDELRT QTSALNRQLA EMEAERDSAT |
| | 1560 1570 1580 1590 1600 |
| | SRARQLQKAV AESEEARRSV DGRLSGVQAE LALQEESVRR SERERRATLD |
| | 1610 1620 1630 1640 1650 |
| | QVATLERSLQ ATESELRASQ EKISKMKANE TKLEGDKRRL KEVLDASESR |
| | 1660 1670 1680 1690 1700 |
| | TVKLELQRRS LEGELQRSRL GLSDREAQAQ ALQDRVDSLQ RQVADSEVKA |
| | 1710 1720 1730 1740 1750 |
| | GTLQLTVERL NGALAKVEES EGALRDKVRG LTEALAQSSA SLNSTRDKNL |
| | 1760 1770 1780 1790 1800 |
| | HLQKALTACE HDRQVLQERL DAARQALSEA RKQSSSIGEQ VQTLRGEVAD |
| | 1810 1820 1830 1840 1850 |
| | LELQRVEAEG QLQQLREVER QRQEGEAAAL NTVQKLQDER RLLQERLGSL |
| | 1860 1870 1880 1890 1900 |
| | QRALAQLEAE KREVERSALR LEKDRVALRR TLDKVEREKL RSHEDTVRLS |
| | 1910 1920 1930 1940 1950 |
| | AEKGRLDRTL TGAELELAEA QRQIQQLEAQ VVVLEQSHSP AQLEVDAQQQ |
| | 1960 1970 1980 1990 2000 |
| | QLELQQEVER IRSAQAQTER TLEARERAHR QRVRGLEEQV STLKGQLQQE |
| | 2010 |
| | LRRSSAPFSP PSGPPEK |
| CSK | Tyrosine-protein kinase CSK (SEQ ID NO: 21) |
| Uniprot | 10 20 30 40 50 |
| P41240 | MSAIQAAWPS GTECIAKYNF HGTAEQDLPF CKGDVLTIVA VIKDPNWYKA |
| | 60 70 80 90 100 |
| | KNKVGREGII PANYVQKREG VKAGTKLSLM PWFHGKITRE QAERLLYPPE |
| | 110 120 130 140 150 |
| | TGLFLVREST NYPGDYTLCV SCDGKVEHYR IMYHASKLSI DEEVYFENLM |
| | 160 170 180 190 200 |
| | QLVEHYTSDA DGLCTRLIKP KVMEGTVAAQ DEFYRSGWAL NMKELKLLQT |
| | 210 220 230 240 250 |
| | IGKGEFGDVM LGDYRGNKVA VKCIKNDATA QAFLAEASVM TQLRHSNEVQ |
| | 260 270 280 290 300 |
| | LLGVIVEEKG GLYIVTEYMA KGSLVDYLRS RGRSVLGGDC LLKESLDVCE |
| | 310 320 330 340 350 |
| | AMEYLEGNNF VHRDLAARNV LVSEDNVAKV SDFGLTKEAS STQDTGKLPV |
| | 360 370 380 390 400 |
| | KWTAPEALRE KKESTKSDVW SFGILLWEIY SFGRVPYPRI PLKDVVPRVE |
| | 410 420 430 440 450 |
| | KGYKMDAPDG CPPAVYEVMK NCWHLDAAMR PSFLQLREQL EHIKTHELHL |
| CUX1 | Homeobox protein cut-like 1 (SEQ ID NO: 22) |
| Uniprot | 10 20 30 40 50 |
| P39880 | MICVAGARLK RELDATATVL ANRQDESEQS RKRLIEQSRE FKKNTPEDLR |
| | 60 70 80 90 100 |
| | KQVAPLLKSF QGEIDALSKR SKEAEAAFLN VYKRLIDVPD PVPALDLGQQ |
| | 110 120 130 140 150 |
| | LQLKVQRLHD IETENQKLRE TLEEYNKEFA EVKNQEVTIK ALKEKIREYE |
| | 160 170 180 190 200 |
| | QTLKNQAETI ALEKEQKLQN DEAEKERKLQ ETQMSTTSKL EEAEHKVQSL |
| | 210 220 230 240 250 |
| | QTALEKTRTE LEDLKTKYDE ETTAKADEIE MIMTDLERAN QRAEVAQREA |
| | 260 270 280 290 300 |
| | ETLREQLSSA NHSLQLASQI QKAPDVEQAI EVLTRSSLEV ELAAKEREIA |
| | 310 320 330 340 350 |
| | QLVEDVQRLQ ASLTKLRENS ASQISQLEQQ LSAKNSTLKQ LEEKLKGQAD |
| | 360 370 380 390 400 |
| | YEEVKKELNI LKSMEFAPSE GAGTQDAAKP LEVLLLEKNR SLQSENAALR |
| | 410 420 430 440 450 |
| | ISNSDLSGSA RRKGKDQPES RRPGSLPAPP PSQLPRNPGE QASNINGTHQ |
| | 460 470 480 490 500 |
| | FSPAGLSQDF FSSSLASPSL PLASTGKFAL NSLLQRQLMQ SFYSKAMQEA |
| | 510 520 530 540 550 |
| | GSTSMIESTG PYSTNSISSQ SPLQQSPDVN GMAPSPSQSE SAGSVSEGEE |
| | 560 570 580 590 600 |
| | MDTAEIARQV KEQLIKHNIG QRIFGHYVLG LSQGSVSEIL ARPKPWNKLT |
| | 610 620 630 640 650 |
| | VRGKEPFHKM KQFLSDEQNI LALRSIQGRQ RENPGQSLNR LFQEVPKRRN |
| | 660 670 680 690 700 |
| | GSEGNITTRI RASETGSDEA IKSILEQAKR ELQVQKTAEP AQPSSASGSG |
| | 710 720 730 740 750 |
| | NSDDAIRSIL QQARREMEAQ QAALDPALKQ APLSQSDITI LTPKLLSTSP |
| | 760 770 780 790 800 |
| | MPTVSSYPPL AISLKKPSAA PEAGASALPN PPALKKEAQD APGLDPQGAA |
| | 810 820 830 840 850 |
| | DCAQGVLRQV KNEVGRSGAW KDHWWSAVQP ERRNAASSEE AKAEETGGGK |
| | 860 870 880 890 900 |
| | EKGSGGSGGG SQPRAERSQL QGPSSSEYWK EWPSAESPYS QSSELSLTGA |
| | 910 920 930 940 950 |
| | SRSETPQNSP LPSSPIVPMS KPTKPSVPPL TPEQYEVYMY QEVDTIELTR |
| | 960 970 980 990 1000 |
| | QVKEKLAKNG ICQRIFGEKV LGLSQGSVSD MLSRPKPWSK LTQKGREPFI |
| | 1010 1020 1030 1040 1050 |
| | RMQLWINGEL GQGVLPVQGQ QQGPVLHSVT SLQDPLQQGC VSSESTPKTS |
| | 1060 1070 1080 1090 1100 |
| | ASCSPAPESP MSSSESVKSL TELVQQPCPP IEASKDSKPP EPSDPPASDS |
| | 1110 1120 1130 1140 1150 |
| | QPTTPLPLSG HSALSIQELV AMSPELDTYG ITKRVKEVLT DNNLGQRLEG |
| | 1160 1170 1180 1190 1200 |
| | ETILGLTQGS VSDLLARPKP WHKLSLKGRE PEVRMQLWLN DPNNVEKIMD |
| | 1210 1220 1230 1240 1250 |
| | MKRMEKKAYM KRRHSSVSDS QPCEPPSVGT EYSQGASPQP QHQLKKPRVV |
| | 1260 1270 1280 1290 1300 |
| | LAPEEKEALK RAYQQKPYPS PKTIEDLATQ LNLKTSTVIN WEHNYRSRIR |
| | 1310 1320 1330 1340 1350 |
| | RELFIEEIQA GSQGQAGASD SPSARSGRAA PSSEGDSCDG VEATEGPGSA |
| | 1360 1370 1380 1390 1400 |
| | DTEEPKSQGE AEREEVPRPA EQTEPPPSGT PGPDDARDDD HEGGPVEGPG |
| | 1410 1420 1430 1440 1450 |
| | PLPSPASATA TAAPAAPEDA ATSAAAAPGE GPAAPSSAPP PSNSSSSSAP |
| | 1460 1470 1480 1490 1500 |
| | RRPSSLQSLF GLPEAAGARD SRDNPLRKKK AANLNSIIHR LEKAASREEP |
| | IEWEF |
| CX3CL1 | Fractalkine (SEQ ID NO: 23) |
| Uniprot | 10 20 30 40 50 |
| P78423 | MAPISLSWLL RLATFCHLTV LLAGQHHGVT KCNITCSKMT SKIPVALLIH |
| | 60 70 80 90 100 |
| | YQQNQASCGK RAIILETRQH RIFCADPKEQ WVKDAMQHLD RQAAALTRNG |
| | 110 120 130 140 150 |
| | GTFEKQIGEV KPRTTPAAGG MDESVVLEPE ATGESSSLEP TPSSQEAQRA |
| | 160 170 180 190 200 |
| | LGTSPELPTG VTGSSGTRLP PTPKAQDGGP VGTELFRVPP VSTAATWQSS |
| | 210 220 230 240 250 |
| | APHQPGPSLW AEAKTSEAPS TQDPSTQAST ASSPAPEENA PSEGQRVWGQ |
| | 260 270 280 290 300 |
| | GQSPRPENSL EREEMGPVPA HTDAFQDWGP GSMAHVSVVP VSSEGTPSRE |
| | 310 320 330 340 350 |
| | PVASGSWTPK AEEPIHATMD PQRLGVLITP VPDAQAATRR QAVGLLAFLG |
| | 360 370 380 390 |
| | LLFCLGVAMF TYQSLQGCPR KMAGEMAEGL RYIPRSCGSN SYVLVPV |
| CYP2S1 | Cytochrome P450 2S1 (SEQ ID NO: 24) |
| Uniprot | 10 20 30 40 50 |
| Q96SQ9 | MEATGTWALL LALALLLLLT LALSGTRARG HLPPGPTPLP LLGNLLQLRP |
| | 60 70 80 90 100 |
| | GALYSGLMRL SKKYGPVFTI YLGPWRPVVV LVGQEAVREA LGGQAEEFSG |
| | 110 120 130 140 150 |
| | RGTVAMLEGT FDGHGVFFSN GERWRQLRKF TMLALRDLGM GKREGEELIQ |
| | 160 170 180 190 200 |
| | AEARCLVETF QGTEGRPEDP SLLLAQATSN VVCSLLFGLR FSYEDKEFQA |
| | 210 220 230 240 250 |
| | VVRAAGGTLL GVSSQGGQTY EMFSWFLRPL PGPHKQLLHH VSTLAAFTVR |
| | 260 270 280 290 300 |
| | QVQQHQGNLD ASGPARDLVD AFLLKMAQEE QNPGTEFINK NMLMTVIYLL |
| | 310 320 330 340 350 |
| | FAGTMTVSTT VGYTLLLLMK YPHVQKWVRE ELNRELGAGQ APSLGDRTRL |
| | 360 370 380 390 400 |
| | PYTDAVLHEA QRLLALVPMG IPRTLMRTTR ERGYTLPQGT EVFPLLGSIL |
| | 410 420 430 440 450 |
| | HDPNIFKHPE EFNPDRELDA DGRERKHEAF LPFSLGKRVC LGEGLAKAEL |
| | 460 470 480 490 500 |
| | FLFFTTILQA FSLESPCPPD TLSLKPTVSG LENIPPAFQL QVRPTDLHST |
| | TQTR |
| DEF6 | Differentially expressed in FDCP 6 homolog (SEQ ID NO: 25) |
| Uniprot | 10 20 30 40 50 |
| Q9H4E7 | MALRKELLKS IWYAFTALDV EKSGKVSKSQ LKVLSHNLYT VLHIPHDPVA |
| | 60 70 80 90 100 |
| | LEEHERDDDD GPVSSQGYMP YLNKYILDKV EEGAFVKEHF DELCWTLTAK |
| | 110 120 130 140 150 |
| | KNYRADSNGN SMLSNQDAFR LWCLENFLSE DKYPLIMVPD EVEYLLKKVL |
| | 160 170 180 190 200 |
| | SSMSLEVSLG ELEELLAQEA QVAQTTGGLS VWQFLELENS GRCLRGVGRD |
| | 210 220 230 240 250 |
| | TLSMAIHEVY QELIQDVLKQ GYLWKRGHLR RNWAERWFQL QPSCLCYFGS |
| | 260 270 280 290 300 |
| | EECKEKRGII PLDAHCCVEV LPDRDGKRCM FCVKTANRTY EMSASDTRQR |
| | 310 320 330 340 350 |
| | QEWTAAIQMA IRLQAEGKTS LHKDLKQKRR EQREQRERRR AAKEEELLRL |
| | 360 370 380 390 400 |
| | QQLQEEKERK LQELELLQEA QRQAERLIQE EEERRRSQHR ELQQALEGQL |
| | 410 420 430 440 450 |
| | REAEQARASM QAEMELKEEE AARQRQRIKE LEEMQQRLQE ALQLEVKARR |
| | 460 470 480 490 500 |
| | DEESVRIAQT RLLEEEEEKL KQLMQLKEEQ ERYIERAQQE KEELQQEMAQ |
| | 510 520 530 540 550 |
| | QSRSLQQAQQ QLEEVRQNRQ RADEDVEAAQ RKLRQASTNV KHWNVQMNRL |
| | 560 570 580 590 600 |
| | MHPIEPGDKR PVTSSSFSGF QPPLLAHRDS SLKRLTRWGS QGNRTPSPNS |
| | 610 620 630 |
| | NEQQKSINGG DEAPAPASTP QEDKLDPAPE N |
| DKK3 | Dickkopf-related protein 3 (SEQ ID NO: 26) |
| Uniprot | 10 20 30 40 50 |
| Q9UBP4 | MQRLGATLLC LLLAAAVPTA PAPAPTATSA PVKPGPALSY PQEEATLNEM |
| | 60 70 80 90 100 |
| | FREVEELMED TQHKLRSAVE EMEAEEAAAK ASSEVNLANL PPSYHNETNT |
| | 110 120 130 140 150 |
| | DTKVGNNTIH VHREIHKITN NQTGQMVESE TVITSVGDEE GRRSHECIID |
| | 160 170 180 190 200 |
| | EDCGPSMYCQ FASFQYTCQP CRGQRMLCTR DSECCGDQLC VWGHCTKMAT |
| | 210 220 230 240 250 |
| | RGSNGTICDN QRDCQPGLCC AFQRGLLFPV CTPLPVEGEL CHDPASRLLD |
| | 260 270 280 290 300 |
| | LITWELEPDG ALDRCPCASG LLCQPHSHSL VYVCKPTFVG SRDQDGEILL |
| | 310 320 330 340 350 |
| | PREVPDEYEV GSFMEEVRQE LEDLERSLTE EMALREPAAA AAALLGGEEI |
| ECH1 | Delta(3,5)-Delta(2,4)-dienoyl-CoA isomerase, mitochondrial (SEQ ID |
| Uniprot | NO: 27) |
| Q13011 | 10 20 30 40 50 |
| | MAAGIVASRR LRDLLTRRLT GSNYPGLSIS LRLTGSSAQE EASGVALGEA |
| | 60 70 80 90 100 |
| | PDHSYESLRV TSAQKHVLHV QLNRPNKRNA MNKVEWREMV ECENKISRDA |
| | 110 120 130 140 150 |
| | DCRAVVISGA GKMFTAGIDL MDMASDILQP KGDDVARISW YLRDIITRYQ |
| | 160 170 180 190 200 |
| | ETENVIERCP KPVIAAVHGG CIGGGVDLVT ACDIRYCAQD AFFQVKEVDV |
| | 210 220 230 240 250 |
| | GLAADVGTLQ RLPKVIGNQS LVNELAFTAR KMMADEALGS GLVSRVEPDK |
| | 260 270 280 290 300 |
| | EVMLDAALAL AAEISSKSPV AVQSTKVNLL YSRDHSVAES LNYVASWNMS |
| | 310 320 |
| | MLQTQDLVKS VQATTENKEL KTVTESKL |
| ENO3 | Beta-enolase (SEQ ID NO: 28) |
| Uniprot | 10 20 30 40 50 |
| P13929 | MAMQKIFARE ILDSRGNPTV EVDLHTAKGR FRAAVPSGAS TGIYEALELR |
| | 60 70 80 90 100 |
| | DGDKGRYLGK GVLKAVENIN NTLGPALLQK KLSVVDQEKV DKEMIELDGT |
| | 110 120 130 140 150 |
| | ENKSKFGANA ILGVSLAVCK AGAAEKGVPL YRHIADLAGN PDLILPVPAF |
| | 160 170 180 190 200 |
| | NVINGGSHAG NKLAMQEFMI LPVGASSFKE AMRIGAEVYH HLKGVIKAKY |
| | 210 220 230 240 250 |
| | GKDATNVGDE GGFAPNILEN NEALELLKTA IQAAGYPDKV VIGMDVAASE |
| | 260 270 280 290 300 |
| | FYRNGKYDLD EKSPDDPARH ITGEKLGELY KSFIKNYPVV SIEDPFDQDD |
| | 310 320 330 340 350 |
| | WATWTSFLSG VNIQIVGDDL TVINPKRIAQ AVEKKACNCL LLKVNQIGSV |
| | 360 370 380 390 400 |
| | TESIQACKLA QSNGWGVMVS HRSGETEDTF IADLVVGLCT GQIKTGAPCR |
| | 410 420 430 |
| | SERLAKYNQL MRIEEALGDK AIFAGRKERN PKAK |
| EPHB3 | Ephrin type-B receptor 3 (SEQ ID NO: 29) |
| Uniprot | 10 20 30 40 50 |
| P54753 | MARARPPPPP SPPPGLLPLL PPLLLLPLLL LPAGCRALEE TLMDTKWVTS |
| | 60 70 80 90 100 |
| | ELAWTSHPES GWEEVSGYDE AMNPIRTYQV CNVRESSQNN WLRTGFIWRR |
| | 110 120 130 140 150 |
| | DVQRVYVELK FTVRDCNSIP NIPGSCKETF NLFYYEADSD VASASSPEWM |
| | 160 170 180 190 200 |
| | ENPYVKVDTI APDESFSRLD AGRVNTKVRS FGPLSKAGFY LAFQDQGACM |
| | 210 220 230 240 250 |
| | SLISVRAFYK KCASTTAGFA LFPETLTGAE PTSLVIAPGT CIPNAVEVSV |
| | 260 270 280 290 300 |
| | PLKLYCNGDG EWMVPVGACT CATGHEPAAK ESQCRPCPPG SYKAKQGEGP |
| | 310 320 330 340 350 |
| | CLPCPPNSRT TSPAASICTC HNNFYRADSD SADSACTTVP SPPRGVISNV |
| | 360 370 380 390 400 |
| | NETSLILEWS EPRDIGGRDD LLYNVICKKC HGAGGASACS RCDDNVEEVP |
| | 410 420 430 440 450 |
| | RQLGLTERRV HISHLLAHTR YTFEVQAVNG VSGKSPLPPR YAAVNITTNQ |
| | 460 470 480 490 500 |
| | AAPSEVPTLR LHSSSGSSLT LSWAPPERPN GVILDYEMKY FEKSEGIAST |
| | 510 520 530 540 550 |
| | VTSQMNSVQL DGLRPDARYV VQVRARTVAG YGQYSRPAEF ETTSERGSGA |
| | 560 570 580 590 600 |
| | QQLQEQLPLI VGSATAGIVE VVAVVVIAIV CLRKQRHGSD SEYTEKLQQY |
| | 610 620 630 640 650 |
| | IAPGMKVYID PETYEDPNEA VREFAKEIDV SCVKIEEVIG AGEFGEVCRG |
| | 660 670 680 690 700 |
| | RLKQPGRREV FVAIKTLKVG YTERQRRDEL SEASIMGQFD HPNIIRLEGV |
| | 710 720 730 740 750 |
| | VIKSRPVMIL TEFMENCALD SFLRLNDGQF TVIQLVGMLR GIAAGMKYLS |
| | 760 770 780 790 800 |
| | EMNYVHRDLA ARNILVNSNL VCKVSDEGLS RFLEDDPSDP TYTSSLGGKI |
| | 810 820 830 840 850 |
| | PIRWTAPEAI AYRKFTSASD VWSYGIVMWE VMSYGERPYW DMSNQDVINA |
| | 860 870 880 890 900 |
| | VEQDYRLPPP MDCPTALHQL MLDCWVRDRN LRPKESQIVN TLDKLIRNAA |
| | 910 920 930 940 950 |
| | SLKVIASAQS GMSQPLLDRT VPDYTTETTV GDWLDAIKMG RYKESFVSAG |
| | 960 970 980 990 |
| | FASEDLVAQM TAEDLLRIGV TLAGHQKKIL SSIQDMRLQM NQTLPVQV |
| FAM116B | DENN domain-containing protein 6B (DENND6B or FAM116B) (SEQ ID NO: 30) |
| Uniprot | 10 20 30 40 50 |
| Q8NEG7 | MDALLGTGPR RARGCLGAAG PTSSGRAART PAAPWARFSA WLECVCVVTF |
| | 60 70 80 90 100 |
| | DLELGQALEL VYPNDERLTD KEKSSICYLS FPDSHSGCLG DTQFSERMRQ |
| | 110 120 130 140 150 |
| | CGGQRSPWHA DDRHYNSRAP VALQREPAHY FGYVYFRQVK DSSVKRGYFQ |
| | 160 170 180 190 200 |
| | KSLVLVSRLP FVRLFQALLS LIAPEYFDKL APCLEAVCSE IDQWPAPAPG |
| | 210 220 230 240 250 |
| | QTLNLPVMGV VVQVRIPSRV DKSESSPPKQ FDQENLLPAP VVLASVHELD |
| | 260 270 280 290 300 |
| | LERCFRPVLT HMQTIWELML LGEPLLVLAP SPDVSSEMVL ALTSCLQPLR |
| | 310 320 330 340 350 |
| | FCCDERPYFT IHDSEFKEFT TRTQAPPNVV LGVINPFFIK TLQHWPHILR |
| | 360 370 380 390 400 |
| | VGEPKMSGDL PKQVKLKKPS RIKTLDTKPG LYTAYTAHLH RDKALLKRLL |
| | 410 420 430 440 450 |
| | KGVQKKRPSD VQSALLRRHL LELTQSFIIP LEHYMASLMP LQKSITPWKT |
| | 460 470 480 490 500 |
| | PPQIQPESQD DELRSLEHAG PQLTCILKGD WLGLYRREFK SPHEDGWYRQ |
| | 510 520 530 540 550 |
| | RHKEMALKLE ALHLEAICEA NIETWMKDKS EVEVVDLVLK LREKLVRAQG |
| | 560 570 580 |
| | HQLPVKEATL QRAQLYIETV IGSLPKDLQA VLCPP |
| FAM46B | Terminal nucleotidyltransferase 5B (TENT5B or FAM46B) (SEQ ID NO: 31) |
| Uniprot | 10 20 30 40 50 |
| Q96A09 | MMPSESGAER RDRAAAQVGT AAATAVATAA PAGGGPDPEA LSAFPGRHLS |
| | 60 70 80 90 100 |
| | GLSWPQVKRL DALLSEPIPI HGRGNEPTLS VQPRQIVQVV RSTLEEQGLH |
| | 110 120 130 140 150 |
| | VHSVRLHGSA ASHVLHPESG LGYKDLDLVF RVDLRSEASE QLTKAVVLAC |
| | 160 170 180 190 200 |
| | LLDELPAGVS RAKITPLTLK EAYVQKLVKV CTDSDRWSLI SLSNKSGKNV |
| | 210 220 230 240 250 |
| | ELKFVDSVRR QFEFSIDSFQ IILDSLLLFG QCSSTPMSEA FHPTVTGESL |
| | 260 270 280 290 300 |
| | YGDFTEALEH LRHRVIATRS PEEIRGGGLL KYCHLLVRGE RPRPSTDVRA |
| | 310 320 330 340 350 |
| | LQRYMCSRFF IDFPDLVEQR RTLERYLEAH FGGADAARRY ACLVTLHRVV |
| | 360 370 380 390 400 |
| | NESTVCLMNH ERRQTLDLIA ALALQALAEQ GPAATAALAW RPPGTDGVVP |
| | 410 420 |
| | ATVNYYVTPV QPLLAHAYPT WLPCN |
| FCHO1 | F-BAR domain only protein 1 (SEQ ID NO: 32) |
| Uniprot | 10 20 30 40 50 |
| O14526 | MSYFGEHEWG EKNHGFEVLY HSVKQGPIST KELADFIRER ATIEETYSKA |
| | 60 70 80 90 100 |
| | MAKLSKLASN GTPMGTFAPL WEVERVSSDK LALCHLELTR KLQDLIKDVL |
| | 110 120 130 140 150 |
| | RYGEEQLKTH KKCKEEVVST LDAVQVLSGV SQLLPKSREN YLNRCMDQER |
| | 160 170 180 190 200 |
| | LRRESTSQKE MDKAETKTKK AAESLRRSVE KYNSARADFE QKMLDSALRE |
| | 210 220 230 240 250 |
| | QAMEETHLRH MKALLGSYAH SVEDTHVQIG QVHEEFKQNI ENVSVEMLLR |
| | 260 270 280 290 300 |
| | KFAESKGTGR EKPGPLDFEA YSAAALQEAM KRLRGAKAFR LPGLSRRERE |
| | 310 320 330 340 350 |
| | PEPPAAVDFL EPDSGTCPEV DEEGFTVRPD VTQNSTAEPS RESSSDSDED |
| | 360 370 380 390 400 |
| | DEEPRKFYVH IKPAPARAPA CSPEAAAAQL RATAGSLILP PGPGGTMKRH |
| | 410 420 430 440 450 |
| | SSRDAAGKPQ RPRSAPRTSS CAERLQSEEQ VSKNLEGPPL ESAFDHEDET |
| | 460 470 480 490 500 |
| | GSSSLGFTSS PSPFSSSSPE NVEDSGLDSP SHAAPGPSPD SWVPRPGTPQ |
| | 510 520 530 540 550 |
| | SPPSCRAPPP EARGIRAPPL PDSPQPLASS PGPWGLEALA GGDLMPAPAD |
| | 560 570 580 590 600 |
| | PTAREGLAAP PRRIRSRKVS CPLTRSNGDL SRSLSPSPLG SSAASTALER |
| | 610 620 630 640 650 |
| | PSFLSQTGHG VSRGPSPVVL GSQDALPIAT AFTEYVHAYF RGHSPSCLAR |
| | 660 670 680 690 700 |
| | VTGELTMTEP AGIVRVFSGT PPPPVISERL VHTTAIEHFQ PNADLLESDP |
| | 710 720 730 740 750 |
| | SQSDPETKDF WINMAALTEA LQRQAEQNPT ASYYNVVLLR YQFSRPGPQS |
| | 760 770 780 790 800 |
| | VPLQLSAHWQ CGATLTQVSV EYGYRPGATA VPTPLINVQI LLPVGEPVIN |
| | 810 820 830 840 850 |
| | VRLQPAATWN LEEKRLTWRL PDVSEAGGSG RLSASWEPLS GPSTPSPVAA |
| | 860 870 880 |
| | QFTSEGTTLS GVDLELVGSG YRMSLVKRRF ATGMYLVSC |
| FGF1 | Fibroblast growth factor 1 (SEQ ID NO: 33) |
| Uniprot | 10 20 30 40 50 |
| P05230 | MAEGEITTFT ALTEKENLPP GNYKKPKLLY CSNGGHELRI LPDGTVDGTR |
| | 60 70 80 90 100 |
| | DRSDQHIQLQ LSAESVGEVY IKSTETGQYL AMDTDGLLYG SQTPNEECLF |
| | 110 120 130 140 150 |
| | LERLEENHYN TYISKKHAEK NWFVGLKKNG SCKRGPRTHY GQKAILFLPL |
| | PVSSD |
| FIBCD1 | Fibrinogen C domain-containing protein 1 (SEQ ID NQ: 34) |
| Uniprot | 10 20 30 40 50 |
| Q8N539 | MVNDRWKTMG GAAQLEDRPR DKPQRPSCGY VLCTVLLALA VLLAVAVTGA |
| | 60 70 80 90 100 |
| | VLFLNHAHAP GTAPPPVVST GAASANSALV TVERADSSHL SILIDPRCPD |
| | 110 120 130 140 150 |
| | LTDSFARLES AQASVLQALT EHQAQPRLVG DQEQELLDTL ADQLPRLLAR |
| | 160 170 180 190 200 |
| | ASELQTECMG LRKGHGTLGQ GLSALQSEQG RLIQLLSESQ GHMAHLVNSV |
| | 210 220 230 240 250 |
| | SDILDALQRD RGLGRPRNKA DLQRAPARGT RPRGCATGSR PRDCLDVLLS |
| | 260 270 280 290 300 |
| | GQQDDGVYSV FPTHYPAGEQ VYCDMRTDGG GWTVFQRRED GSVNEFRGWD |
| | 310 320 330 340 350 |
| | AYRDGFGRLT GEHWIGLKRI HALTTQAAYE LHVDLEDFEN GTAYARYGSF |
| | 360 370 380 390 400 |
| | GVGLESVDPE EDGYPLTVAD YSGTAGDSLL KHSGMRETTK DRDSDHSENN |
| | 410 420 430 440 450 |
| | CAAFYRGAWW YRNCHTSNLN GQYLRGAHAS YADGVEWSSW TGWQYSLKES |
| | 460 |
| | EMKIRPVRED R |
| FZD2 | Frizzled-2 (SEQ ID NO: 35) |
| Uniprot | 10 20 30 40 50 |
| Q14332 | MRPRSALPRL LLPLLLLPAA GPAQFHGEKG ISIPDHGFCQ PISIPLCTDI |
| | 60 70 80 90 100 |
| | AYNQTIMPNL LGHTNQEDAG LEVHQFYPLV KVQCSPELRF FLCSMYAPVC |
| | 110 120 130 140 150 |
| | TVLEQAIPPC RSICERARQG CEALMNKFGF QWPERLRCEH FPRHGAEQIC |
| | 160 170 180 190 200 |
| | VGQNHSEDGA PALLTTAPPP GLQPGAGGTP GGPGGGGAPP RYATLEHPFH |
| | 210 220 230 240 250 |
| | CPRVLKVPSY LSYKELGERD CAAPCEPARP DGSMFFSQEE TRFARLWILT |
| | 260 270 280 290 300 |
| | WSVLCCASTF FTVTTYLVDM QRFRYPERPI IFLSGCYTMV SVAYIAGFVL |
| | 310 320 330 340 350 |
| | QERVVCNERF SEDGYRTVVQ GTKKEGCTIL FMMLYFFSMA SSIWWVILSL |
| | 360 370 380 390 400 |
| | TWFLAAGMKW GHEAIEANSQ YFHLAAWAVP AVKTITILAM GQIDGDLLSG |
| | 410 420 430 440 450 |
| | VCFVGLNSLD PLRGFVLAPL FVYLFIGTSF LLAGFVSLFR IRTIMKHDGT |
| | 460 470 480 490 500 |
| | KTEKLERLMV RIGVESVLYT VPATIVIACY FYEQAFREHW ERSWVSQHCK |
| | 510 520 530 540 550 |
| | SLAIPCPAHY TPRMSPDFTV YMIKYLMTLI VGITSGFWIW SGKTLHSWRK |
| | 560 |
| | FYTRLTNSRH GETTV |
| GDPD5 | Glycerophosphodiester phosphodiesterase domain-containing protein 5 |
| Uniprot | (SEQ ID NO: 36) |
| Q8WTR4 | 10 20 30 40 50 |
| | MVRHQPLQYY EPQLCLSCLT GIYGCRWKRY QRSHDDTTPW ERLWELLLTF |
| | 60 70 80 90 100 |
| | TFGLTLTWLY FWWEVHNDYD EENWYLYNRM GYWSDWPVPI LVTTAAAFAY |
| | 110 120 130 140 150 |
| | IAGLLVLALC HIAVGQQMNL HWLHKIGLVV ILASTVVAMS AVAQLWEDEW |
| | 160 170 180 190 200 |
| | EVLLISLQGT APFLHVGAVA AVTMLSWIVA GQFARAERTS SQVTILCTFF |
| | 210 220 230 240 250 |
| | TVVFALYLAP LTISSPCIME KKDLGPKPAL IGHRGAPMLA PEHTIMSERK |
| | 260 270 280 290 300 |
| | ALEQKLYGLQ ADITISLDGV PELMHDTTER RTINVEEEFP ELARRPASML |
| | 310 320 330 340 350 |
| | NWTTLQRENA GQWELKTDPF WTASSISPSD HREAQNQSIC SLAELLELAK |
| | 360 370 380 390 400 |
| | GNATLLLNLR DPPREHPYRS SFINVTLEAV LHSGFPQHQV MWLPSRQRPL |
| | 410 420 430 440 450 |
| | VRKVAPGFQQ TSGSKEAVAS LRRGHIQRLN LRYTQVSRQE LRDYASWNLS |
| | 460 470 480 490 500 |
| | VNLYTVNAPW LESLIWCAGV PSVTSDNSHA LSQVPSPLWI MPPDEYCLMW |
| | 510 520 530 540 550 |
| | VTADLVSFTL IVGIFVLQKW RIGGIRSYNP EQIMLSAAVR RTSRDVSIMK |
| | 560 570 580 590 600 |
| | EKLIFSEISD GVEVSDVLSV CSDNSYDTYA NSTATPVGPR GGGSHTKTLI |
| | ERSGR |
| GPR56 | Adhesion G-protein coupled receptor G1 (SEQ ID NO: 37) |
| Uniprot | 10 20 30 40 50 |
| Q9Y653 | MTPQSLLQTT LFLLSLLFLV QGAHGRGHRE DERFCSQRNQ THRSSLHYKP |
| | 60 70 80 90 100 |
| | TPDLRISIEN SEEALTVHAP FPAAHPASRS FPDPRGLYHF CLYWNRHAGR |
| | 110 120 130 140 150 |
| | LHLLYGKRDF LLSDKASSLL CFQHQEESLA QGPPLLATSV TSWWSPQNIS |
| | 160 170 180 190 200 |
| | LPSAASETFS FHSPPHTAAH NASVDMCELK RDLQLLSQFL KHPQKASRRP |
| | 210 220 230 240 250 |
| | SAAPASQQLQ SLESKLTSVR FMGDMVSFEE DRINATVWKL QPTAGLQDLH |
| | 260 270 280 290 300 |
| | IHSRQEEEQS EIMEYSVLLP RTLFQRTKGR SGEAEKRLLL VDESSQALFQ |
| | 310 320 330 340 350 |
| | DKNSSQVLGE KVLGIVVQNT KVANLTEPVV LTFQHQLQPK NVTLQCVEWV |
| | 360 370 380 390 400 |
| | EDPTLSSPGH WSSAGCETVR RETQTSCFCN HLTYFAVLMV SSVEVDAVHK |
| | 410 420 430 440 450 |
| | HYLSLLSYVG CVVSALACLV TIAAYICSRV PLPCRRKPRD YTIKVHMNLL |
| | 460 470 480 490 500 |
| | LAVELLDTSE LLSEPVALTG SEAGCRASAI FLHFSLLTCL SWMGLEGYNL |
| | 510 520 530 540 550 |
| | YRIVVEVEGT YVPGYLLKLS AMGWGFPIFL VTLVALVDVD NYGPIILAVH |
| | 560 570 580 590 600 |
| | RTPEGVIYPS MCWIRDSLVS YITNLGLESE VELENMAMLA TMVVQILRLR |
| | 610 620 630 640 650 |
| | PHTQKWSHVL TLLGLSLVLG LPWALIFFSF ASGTFQLVVL YLFSIITSFQ |
| | 660 670 680 690 |
| | GFLIFIWYWS MRLQARGGPS PLKSNSDSAR LPISSGSTSS SRI |
| HDAC11 | Histone deacetylase 11 (SEQ ID NO: 38) |
| Uniprot | 10 20 30 40 50 |
| Q96DB2 | MLHTTQLYQH VPETRWPIVY SPRYNITEMG LEKLHPEDAG KWGKVINELK |
| | 60 70 80 90 100 |
| | EEKLLSDSML VEAREASEED LLVVHTRRYL NELKWSFAVA TITEIPPVIE |
| | 110 120 130 140 150 |
| | LPNELVQRKV LRPLRTQTGG TIMAGKLAVE RGWAINVGGG FHHCSSDRGG |
| | 160 170 180 190 200 |
| | GFCAYADITL AIKFLFERVE GISRATIIDL DAHQGNGHER DFMDDKRVYI |
| | 210 220 230 240 250 |
| | MDVYNRHIYP GDREAKQAIR RKVELEWGTE DDEYLDKVER NIKKSLQEHL |
| | 260 270 280 290 300 |
| | PDVVVYNAGT DILEGDRIGG LSISPAGIVK RDELVERMVR GRRVPILMVT |
| | 310 320 330 340 |
| | SGGYQKRTAR IIADSILNLF GLGLIGPESP SVSAQNSDTP LLPPAVP |
| HSA011916 | CTD nuclear envelope phosphatase 1 (CTDNEP1 or DULLARD) |
| Uniprot | (SEQ ID NO: 39) |
| O95476 | 10 20 30 40 50 |
| | MMRTQCLLGL RTFVAFAAKL WSFFIYLLRR QIRTVIQYQT VRYDILPLSP |
| | 60 70 80 90 100 |
| | VSRNRLAQVK RKILVLDLDE TLIHSHHDGV LRPTVRPGTP PDFILKVVID |
| | 110 120 130 140 150 |
| | KHPVRFFVHK RPHVDEFLEV VSQWYELVVF TASMEIYGSA VADKLDNSRS |
| | 160 170 180 190 200 |
| | ILKRRYYRQH CTLELGSYIK DLSVVHSDLS SIVILDNSPG AYRSHPDNAI |
| | 210 220 230 240 |
| | PIKSWFSDPS DTALLNLLPM LDALRFTADV RSVLSRNLHQ HRLW |
| ID3 | DNA-binding protein inhibitor ID-3 (SEQ ID NO: 40) |
| Uniprot | 10 20 30 40 50 |
| Q02535 | MKALSPVRGC YEAVCCLSER SLAIARGRGK GPAAEEPLSL LDDMNHCYSR |
| | 60 70 80 90 100 |
| | LRELVPGVPR GTQLSQVEIL QRVIDYILDL QVVLAEPAPG PPDGPHLPIQ |
| | 110 |
| | TAELTPELVI SNDKRSFCH |
| IER5L | Immediate early response gene 5-like protein (SEQ ID NO: 41) |
| Uniprot | 10 20 30 40 50 |
| QST953 | MECALDAQSL ISISLRKIHS SRTQRGGIKL HKNLLVSYVL RNARQLYLSE |
| | 60 70 80 90 100 |
| | RYAELYRRQQ QQQQQQPPHH QHQHLAYAAP GMPASAADEG PLQLGGGGDA |
| | 110 120 130 140 150 |
| | EAREPAARHQ LHQLHQLHQL HLQQQLHQHQ HPAPRGCAAA AAAGAPAGGA |
| | 160 170 180 190 200 |
| | GALSELPGCA ALQPPHGAPH RGQPLEPLQP GPAPLPLPLP PPAPAALCPR |
| | 210 220 230 240 250 |
| | DPRAPAACSA PPGAAPPAAA ASPPASPAPA SSPGFYRGAY PTPSDEGLHC |
| | 260 270 280 290 300 |
| | SSQTTVLDLD THVVTTVENG YLHQDCCASA HCPCCGQGAP GPGLASAAGC |
| | 310 320 330 340 350 |
| | KRKYYPGQEE EEDDEEDAGG LGAEPPGGAP FAPCKRARFE DFCPDSSPDA |
| | 360 370 380 390 400 |
| | SNISNLISIF GSGFSGLVSR QPDSSEQPPP LNGQLCAKQA LASLGAWTRA |
| | IVAF |
| IGFBP5 | Insulin-like growth factor-binding protein 5 (SEQ ID NO: 42) |
| Uniprot | 10 20 30 40 50 |
| P24593 | MVLLTAVLLL LAAYAGPAQS LGSFVHCEPC DEKALSMCPP SPLGCELVKE |
| | 60 70 80 90 100 |
| | PGCGCCMTCA LAEGQSCGVY TERCAQGLRC LPRQDEEKPL HALLHGRGVC |
| | 110 120 130 140 150 |
| | LNEKSYREQV KIERDSREHE EPTTSEMAEE TYSPKIFRPK HTRISELKAE |
| | 160 170 180 190 200 |
| | AVKKDRRKKL TQSKFVGGAE NTAHPRIISA PEMRQESEQG PCRRHMEASL |
| | 210 220 230 240 250 |
| | QELKASPRMV PRAVYLPNCD RKGFYKRKQC KPSRGRKRGI CWCVDKYGMK |
| | 260 270 |
| | LPGMEYVDGD FQCHTEDSSN VE |
| IL6 | Interleukin-6 (SEQ ID NO: 43) |
| Uniprot | 10 20 30 40 50 |
| P05231 | MNSESTSAFG PVAFSLGLLL VLPAAFPAPV PPGEDSKDVA APHRQPLTSS |
| | 60 70 80 90 100 |
| | ERIDKQIRYI LDGISALRKE TQNKSNMCES SKEALAENNL NLPKMAEKDG |
| | 110 120 130 140 150 |
| | CFQSGENEET CLVKIITGLL EFEVYLEYLQ NRFESSEEQA RAVQMSTKVL |
| | 160 170 180 190 200 |
| | IQFLQKKAKN LDAITTPDPT TNASLLTKLQ AQNQWLQDMT THLILRSFKE |
| | 210 |
| | FLQSSLRALR QM |
| KRT7 | Keratin, type II cytoskeletal 7 (SEQ ID NO: 44) |
| Uniprot | 10 20 30 40 50 |
| P08729 | MSIHESSPVF TSRSAAFSGR GAQVRLSSAR PGGLGSSSLY GLGASRPRVA |
| | 60 70 80 90 100 |
| | VRSAYGGPVG AGIREVTINQ SLLAPLRLDA DPSLQRVRQE ESEQIKTINN |
| | 110 120 130 140 150 |
| | KFASFIDKVR FLEQQNKLLE TKWILLQEQK SAKSSRLPDI FEAQIAGLRG |
| | 160 170 180 190 200 |
| | QLEALQVDGG RLEAELRSMQ DVVEDEKNKY EDEINHRTAA ENEFVVLKKD |
| | 210 220 230 240 250 |
| | VDAAYMSKVE LEAKVDALND EINFLRTLNE TELTELQSQI SDTSVVLSMD |
| | 260 270 280 290 300 |
| | NSRSLDLDGI IAEVKAQYEE MAKCSRAEAE AWYQTKFETL QAQAGKHGDD |
| | 310 320 330 340 350 |
| | LRNTRNEISE MNRAIQRLQA EIDNIKNQRA KLEAAIAEAE ERGELALKDA |
| | 360 370 380 390 400 |
| | RAKQEELEAA LQRGKQDMAR QLREYQELMS VKLALDIEIA TYRKLLEGEE |
| | 410 420 430 440 450 |
| | SRLAGDGVGA VNISVMNSTG GSSSGGGIGL TLGGTMGSNA LSFSSSAGPG |
| | 460 |
| | LLKAYSIRTA SASRRSARD |
| LAMA5 | Laminin subunit alpha-5 (SEQ ID NO: 45) |
| Uniprot | 10 20 30 40 50 |
| O15230 | MAKRLCAGSA LCVRGPRGPA PLLLVGLALL GAARAREEAG GGFSLHPPYF |
| | 60 70 80 90 100 |
| | NLAEGARIAA SATCGEEAPA RGSPRPTEDL YCKLVGGPVA GGDPNQTIRG |
| | 110 120 130 140 150 |
| | QYCDICTAAN SNKAHPASNA IDGTERWWQS PPLSRGLEYN EVNVTLDLGQ |
| | 160 170 180 190 200 |
| | VFHVAYVLIK FANSPRPDLW VLERSMDEGR TYQPWQFFAS SKRDCLEREG |
| | 210 220 230 240 250 |
| | PQTLERITRD DAAICTTEYS RIVPLENGEI VVSLVNGRPG AMNESYSPLL |
| | 260 270 280 290 300 |
| | REFTKATNVR LRFLRTNTLL GHLMGKALRD PTVTRRYYYS IKDISIGGRC |
| | 310 320 330 340 350 |
| | VCHGHADACD AKDPTDPERL QCTCQHNTCG GTCDRCCPGF NQQPWKPATA |
| | 360 370 380 390 400 |
| | NSANECQSCN CYGHATDCYY DPEVDRRRAS QSLDGTYQGG GVCIDCQHHT |
| | 410 420 430 440 450 |
| | TGVNCERCLP GFYRSPNHPL DSPHVQRRCN CESDETDGTC EDLTGRCYCR |
| | 460 470 480 490 500 |
| | PNFSGERCDV CAEGETGFPS CYPTPSSSND TREQVLPAGQ IVNCDCSAAG |
| | 510 520 530 540 550 |
| | TQGNACRKDP RVGRCLCKPN FQGTHCELCA PGFYGPGCQP CQCSSPGVAD |
| | 560 570 580 590 600 |
| | DRCDPDTGQC RCRVGFEGAT CDRCAPGYFH FPLCQLCGCS PAGTLPEGCD |
| | 610 620 630 640 650 |
| | EAGRCLCQPE FAGPHCDRCR PGYHGFPNCQ ACTCDPRGAL DQLCGAGGLC |
| | 660 670 680 690 700 |
| | RCRPGYTGTA CQECSPGEHG FPSCVPCHCS AEGSLHAACD PRSGQCSCRP |
| | 710 720 730 740 750 |
| | RVTGLRCDTC VPGAYNFPYC EAGSCHPAGL APVDPALPEA QVPCMCRAHV |
| | 760 770 780 790 800 |
| | EGPSCDRCKP GFWGLSPSNP EGCTRCSCDL RGTLGGVAEC QPGTGQCECK |
| | 810 820 830 840 850 |
| | PHVCGQACAS CKDGFFGLDQ ADYFGCRSCR CDIGGALGQS CEPRTGVCRC |
| | 860 870 880 890 900 |
| | RPNTQGPTCS EPARDHYLPD LHHLRLELEE AATPEGHAVR FGENPLEFEN |
| | 910 920 930 940 950 |
| | FSWRGYAQMA PVQPRIVARL NITSPDLFWL VERYVNRGAM SVSGRVSVRE |
| | 960 970 980 990 1000 |
| | EGRSATCANC TAQSQPVAFP PSTEPAFITV PQRGFGEPFV LNPGTWALRV |
| | 1010 1020 1030 1040 1050 |
| | EAEGVLLDYV VLLPSAYYEA ALLQLRVTEA CTYRPSAQQS GDNCLLYTHL |
| | 1060 1070 1080 1090 1100 |
| | PLDGFPSAAG LEALCRQDNS LPRPCPTEQL SPSHPPLITC TGSDVDVQLQ |
| | 1110 1120 1130 1140 1150 |
| | VAVPQPGRYA LVVEYANEDA RQEVGVAVHT PQRAPQQGLL SLHPCLYSTL |
| | 1160 1170 1180 1190 1200 |
| | CRGTARDTQD HLAVFHLDSE ASVRITAEQA RFFLHGVTLV PIEEFSPEFV |
| | 1210 1220 1230 1240 1250 |
| | EPRVSCISSH GAFGPNSAAC LPSREPKPPQ PIILRDCQVI PLPPGLPLTH |
| | 1260 1270 1280 1290 1300 |
| | AQDLTPAMSP AGPRPRPPTA VDPDAEPTLL REPQATVVFT THVPTLGRYA |
| | 1310 1320 1330 1340 1350 |
| | FLLHGYQPAH PTFPVEVLIN AGRVWQGHAN ASFCPHGYGC RTLVVCEGQA |
| | 1360 1370 1380 1390 1400 |
| | LLDVTHSELT VTVRVPKGRW LWLDYVLVVP ENVYSEGYLR EEPLDKSYDF |
| | 1410 1420 1430 1440 1450 |
| | ISHCAAQGYH ISPSSSSLFC RNAAASLSLE YNNGARPCGC HEVGATGPTC |
| | 1460 1470 1480 1490 1500 |
| | EPEGGQCPCH AHVIGRDCSR CATGYWGFPN CRPCDCGARL CDELTGQCIC |
| | 1510 1520 1530 1540 1550 |
| | PPRTIPPDCL LCQPQTFGCH PLVGCEECNC SGPGIQELTD PTCDTDSGQC |
| | 1560 1570 1580 1590 1600 |
| | KCRPNVTGRR CDTCSPGFHG YPRCRPCDCH EAGTAPGVCD PLTGQCYCKE |
| | 1610 1620 1630 1640 1650 |
| | NVQGPKCDQC SLGTESLDAA NPKGCTRCFC FGATERCRSS SYTRQEFVDM |
| | 1660 1670 1680 1690 1700 |
| | EGWVLLSTDR QVVPHERQPG TEMLRADLRH VPEAVPEAFP ELYWQAPPSY |
| | 1710 1720 1730 1740 1750 |
| | LGDRVSSYGG TLRYELHSET QRGDVFVPME SRPDVVLQGN QMSITFLEPA |
| | 1760 1770 1780 1790 1800 |
| | YPTPGHVHRG QLQLVEGNER HTETRNTVSR EELMMVLASL EQLQIRALFS |
| | 1810 1820 1830 1840 1850 |
| | QISSAVFLRR VALEVASPAG QGALASNVEL CLCPASYRGD SCQECAPGFY |
| | 1860 1870 1880 1890 1900 |
| | RDVKGLEIGR CVPCQCHGHS DRCLPGSGVC VDCQHNTEGA HCERCQAGFV |
| | 1910 1920 1930 1940 1950 |
| | SSRDDPSAPC VSCPCPLSVP SNNFAEGCVL RGGRTQCLCK PGYAGASCER |
| | 1960 1970 1980 1990 2000 |
| | CAPGFFGNPL VLGSSCQPCD CSGNGDPNLL FSDCDPLTGA CRGCLRHTTG |
| | 2010 2020 2030 2040 2050 |
| | PRCEICAPGF YGNALLPGNC TRCDCTPCGT EACDPHSGHC LCKAGVTGRR |
| | 2060 2070 2080 2090 2100 |
| | CDRCQEGHFG FDGCGGCRPC ACGPAAEGSE CHPQSGQCHC RPGTMGPQCR |
| | 2110 2120 2130 2140 2150 |
| | ECAPGYWGLP EQGCRRCQCP GGRCDPHTGR CNCPPGLSGE RCDTCSQQHQ |
| | 2160 2170 2180 2190 2200 |
| | VPVPGGPVGH SIHCEVCDHC VVLLLDDLER AGALLPATHE QLRGINASSM |
| | 2210 2220 2230 2240 2250 |
| | AWARLHRINA SIADLQSQLR SPLGPRHETA QQLEVLEQQS TSLGQDARRL |
| | 2260 2270 2280 2290 2300 |
| | GGQAVGTRDQ ASQLLAGTEA TLGHAKTLLA AIRAVDRTLS ELMSQTGHLG |
| | 2310 2320 2330 2340 2350 |
| | LANASAPSGE QLLRTLAEVE RLLWEMRARD LGAPQAAAEA ELAAAQRLLA |
| | 2360 2370 2380 2390 2400 |
| | RVQEQLSSLW EENQALATQT RDRLAQHEAG LMDLREALNR AVDATREAQE |
| | 2410 2420 2430 2440 2450 |
| | LNSRNQERLE EALQRKQELS RDNATLQATL HAARDTLASV FRLLHSLDQA |
| | 2460 2470 2480 2490 2500 |
| | KEELERLAAS LDGARTPLLQ RMQTESPAGS KLRLVEAAEA HAQQLGQLAL |
| | 2510 2520 2530 2540 2550 |
| | NISSIILDVN QDRLTQRAIE ASNAYSRILQ AVQAAEDAAG QALQQADHTW |
| | 2560 2570 2580 2590 2600 |
| | ATVVRQGLVD RAQQLLANST ALEEAMLQEQ QRLGLVWAAL QGARTQLRDV |
| | 2610 2620 2630 2640 2650 |
| | RAKKDQLEAH IQAAQAMLAM DTDETSKKIA HAKAVAAEAQ DTATRVQSQL |
| | 2660 2670 2680 2690 2700 |
| | QAMQENVERW QGQYEGLRGQ DIGQAVLDAG HSVSTLEKTL PQLLAKLSIL |
| | 2710 2720 2730 2740 2750 |
| | ENRGVHNASL ALSASIGRVR ELIAQARGAA SKVKVPMKEN GRSGVQLRTP |
| | 2760 2770 2780 2790 2800 |
| | RDLADLAAYT ALKFYLQGPE PEPGQGTEDR FVMYMGSRQA TGDYMGVSLR |
| | 2810 2820 2830 2840 2850 |
| | DKKVHWVYQL GEAGPAVISI DEDIGEQFAA VSLDRTLQFG HMSVTVERQM |
| | 2860 2870 2880 2890 2900 |
| | IQETKGDTVA PGAEGLLNIR PDDFVEYVGG YPSTFTPPPL LRFPGYRGCI |
| | 2910 2920 2930 2940 2950 |
| | EMDTLNEEVV SLYNFERTFQ LDTAVDRPCA RSKSTGDPWL TDGSYLDGTG |
| | 2960 2970 2980 2990 3000 |
| | FARISFDSQI STTKRFEQEL RLVSYSGVLF FLKQQSQFLC LAVQEGSEVL |
| | 3010 3020 3030 3040 3050 |
| | LYDFGAGLKK AVPLQPPPPL TSASKAIQVF LLGGSRKRVL VRVERATVYS |
| | 3060 3070 3080 3090 3100 |
| | VEQDNDLELA DAYYLGGVPP DQLPPSLRRL FPTGGSVRGC VKGIKALGKY |
| | 3110 3120 3130 3140 3150 |
| | VDLKRINTTG VSAGCTADLL VGRAMTEHGH GELRLALSNV APLTGNVYSG |
| | 3160 3170 3180 3190 3200 |
| | FGFHSAQDSA LLYYRASPDG LCQVSLQQGR VSLQLLRTEV KTQAGFADGA |
| | 3210 3220 3230 3240 3250 |
| | PHYVAFYSNA TGVWLYVDDQ LQQMKPHRGP PPELQPQPEG PPRLLLGGLP |
| | 3260 3270 3280 3290 3300 |
| | ESGTIYNESG CISNVFVQRL LGPQRVEDLQ QNLGSVNVST GCAPALQAQT |
| | 3310 3320 3330 3340 3350 |
| | PGLGPRGLQA TARKASRRSR QPARHPACML PPHLRTTRDS YQFGGSLSSH |
| | 3360 3370 3380 3390 3400 |
| | LEFVGILARH RNWPSLSMHV LPRSSRGLLL FTARLRPGSP SLALFLSNGH |
| | 3410 3420 3430 3440 3450 |
| | FVAQMEGLGT RLRAQSRQRS RPGRWHKVSV RWEKNRILLV TDGARAWSQE |
| | 3460 3470 3480 3490 3500 |
| | GPHRQHQGAE HPQPHTLFVG GLPASSHSSK LPVTVGESGC VKRLRLHGRP |
| | 3510 3520 3530 3540 3550 |
| | LGAPTRMAGV TPCILGPLEA GLFFPGSGGV ITLDLPGATL PDVGLELEVR |
| | 3560 3570 3580 3590 3600 |
| | PLAVIGLIFH EGQARTPPYL QLQVTEKQVL LRADDGAGEF STSVTRPSVL |
| | 3610 3620 3630 3640 3650 |
| | CDGQWHRLAV MKSGNVLRLE VDAQSNHTVG PLLAAAAGAP APLYIGGLPE |
| | 3660 3670 3680 3690 |
| | PMAVQPWPPA YCGCMRRLAV NRSPVAMTRS VEVHGAVGAS GCPAA |
| LIMK2 | LIM domain kinase 2 (SEQ ID NO: 46) |
| Uniprot | 10 20 30 40 50 |
| P53671 | MSALAGEDVW RCPGCGDHIA PSQIWYRTVN ETWHGSCERC SECQDSLTNW |
| | 60 70 80 90 100 |
| | YYEKDGKLYC PKDYWGKEGE FCHGCSLLMT GPEMVAGEFK YHPECFACMS |
| | 110 120 130 140 150 |
| | CKVIIEDGDA YALVQHATLY CGKCHNEVVL APMFERISTE SVQEQLPYSV |
| | 160 170 180 190 200 |
| | TLISMPATTE GRRGFSVSVE SACSNYATTV QVKEVNRMHI SPNNRNAIHP |
| | 210 220 230 240 250 |
| | GDRILEINGT PVRTERVEEV EDAISQTSQT LQLLIEHDPV SQRLDQLRLE |
| | 260 270 280 290 300 |
| | ARLAPHMQNA GHPHALSTED TKENLEGTLR RRSLRRSNSI SKSPGPSSPK |
| | 310 320 330 340 350 |
| | EPLLFSRDIS RSESLRCSSS YSQQIFRPCD LIHGEVLGKG FFGQAIKVTH |
| | 360 370 380 390 400 |
| | KATGKVMVMK ELIRCDEETQ KTELTEVKVM RSLDHPNVLK FIGVLYKDKK |
| | 410 420 430 440 450 |
| | LNLLTEYIEG GTLKDFLRSM DPFPWQQKVR FAKGIASGMA YLHSMCIIHR |
| | 460 470 480 490 500 |
| | DLNSHNCLIK LDKTVVVADF GLSRLIVEER KRAPMEKATT KKRTLRKNDR |
| | 510 520 530 540 550 |
| | KKRYTVVGNP YWMAPEMING KSYDETVDIF SEGIVICEII GQVYADPDCL |
| | 560 570 580 590 600 |
| | PRTLDFGLNV KLFWEKFVPT DCPPAFFPLA AICCRLEPES RPAFSKLEDS |
| | 610 620 630 |
| | FEALSLYLGE LGIPLPAELE ELDHTVSMQY GLTRDSPP |
| LOXL1 | Lysyl oxidase homolog 1 (SEQ ID NO: 47) |
| Uniprot | 10 20 30 40 50 |
| Q08397 | MALARGSRQL GALVWGACLC VLVHGQQAQP GQGSDPARWR QLIQWENNGQ |
| | 60 70 80 90 100 |
| | VYSLINSGSE YVPAGPQRSE SSSRVLLAGA PQAQQRRSHG SPRRRQAPSL |
| | 110 120 130 140 150 |
| | PLPGRVGSDT VRGQARHPFG FGQVPDNWRE VAVGDSTGMA RARTSVSQQR |
| | 160 170 180 190 200 |
| | HGGSASSVSA SAFASTYRQQ PSYPQQFPYP QAPFVSQYEN YDPASRTYDQ |
| | 210 220 230 240 250 |
| | GFVYYRPAGG GVGAGAAAVA SAGVIYPYQP RARYEEYGGG EELPEYPPQG |
| | 260 270 280 290 300 |
| | FYPAPERPYV PPPPPPPDGL DRRYSHSLYS EGTPGFEQAY PDPGPEAAQA |
| | 310 320 330 340 350 |
| | HGGDPRLGWY PPYANPPPEA YGPPRALEPP YLPVRSSDTP PPGGERNGAQ |
| | 360 370 380 390 400 |
| | QGRLSVGSVY RPNQNGRGLP DLVPDPNYVQ ASTYVQRAHL YSLRCAAEEK |
| | 410 420 430 440 450 |
| | CLASTAYAPE ATDYDVRVLL REPQRVKNQG TADFLPNRPR HTWEWHSCHQ |
| | 460 470 480 490 500 |
| | HYHSMDEFSH YDLLDAATGK KVAEGHKASF CLEDSTCDEG NLKRYACTSH |
| | 510 520 530 540 550 |
| | TQGLSPGCYD TYNADIDCQW IDITDVQPGN YILKVHVNPK YIVLESDEIN |
| | 560 570 |
| | NVVRCNIHYT GRYVSATNCK IVQS |
| LOXL2 | Lysyl oxidase homolog 2 (SEQ ID NO: 48) |
| Uniprot | 10 20 30 40 50 |
| Q9Y4K0 | MERPLCSHLC SCLAMLALLS PLSLAQYDSW PHYPEYFQQP APEYHQPQAP |
| | 60 70 80 90 100 |
| | ANVAKIQLRL AGQKRKHSEG RVEVYYDGQW GTVCDDDESI HAAHVVCREL |
| | 110 120 130 140 150 |
| | GYVEAKSWTA SSSYGKGEGP IWLDNLHCTG NEATLAACTS NGWGVTDCKH |
| | 160 170 180 190 200 |
| | TEDVGVVCSD KRIPGFKEDN SLINQIENLN IQVEDIRIRA ILSTYRKRTP |
| | 210 220 230 240 250 |
| | VMEGYVEVKE GKTWKQICDK HWTAKNSRVV CGMFGFPGER TYNTKVYKMF |
| | 260 270 280 290 300 |
| | ASRRKQRYWP FSMDCTGTEA HISSCKLGPQ VSLDPMKNVT CENGLPAVVS |
| | 310 320 330 340 350 |
| | CVPGQVESPD GPSRERKAYK PEQPLVRLRG GAYIGEGRVE VLKNGEWGTV |
| | 360 370 380 390 400 |
| | CDDKWDLVSA SVVCRELGFG SAKEAVTGSR LGQGIGPIHL NEIQCTGNEK |
| | 410 420 430 440 450 |
| | SIIDCKENAE SQGCNHEEDA GVRCNTPAMG LQKKLRINGG RNPYEGRVEV |
| | 460 470 480 490 500 |
| | LVERNGSLVW GMVCGQNWGI VEAMVVCRQL GLGFASNAFQ ETWYWHGDVN |
| | 510 520 530 540 550 |
| | SNKVVMSGVK CSGTELSLAH CRHDGEDVAC PQGGVQYGAG VACSETAPDL |
| | 560 570 580 590 600 |
| | VLNAEMVQQT TYLEDRPMEM LQCAMEENCL SASAAQTDPT TGYRRLLRES |
| | 610 620 630 640 650 |
| | SQIHNNGQSD FRPKNGRHAW IWHDCHRHYH SMEVFTHYDL INLNGTKVAE |
| | 660 670 680 690 700 |
| | GHKASFCLED TECEGDIQKN YECANFGDQG ITMGCWDMYR HDIDCQWVDI |
| | 710 720 730 740 750 |
| | TDVPPGDYLF QVVINPNFEV AESDYSNNIM KQRSRYDGHR IWMYNCHIGG |
| | 760 770 |
| | SFSEETEKKE EHFSGLLNNQ LSPQ |
| LRP1 | Prolow-density lipoprotein receptor-related protein 1 (SEQ ID NO: 49) |
| Uniprot | 10 20 30 40 50 |
| Q07954 | MLTPPLLLLL PLLSALVAAA IDAPKTCSPK QFACRDQITC ISKGWRCDGE |
| | 60 70 80 90 100 |
| | RDCPDGSDEA PEICPQSKAQ RCQPNEHNCE GTELCVPMSR LCNGVQDCMD |
| | 110 120 130 140 150 |
| | GSDEGPHCRE LQGNCSRLGC QHHCVPTLDG PTCYCNSSFQ LQADGKTCKD |
| | 160 170 180 190 200 |
| | FDECSVYGTC SQLCTNTDGS FICGCVEGYL LQPDNRSCKA KNEPVDRPPV |
| | 210 220 230 240 250 |
| | LLIANSQNIL ATYLSGAQVS TITPTSTRQT TAMDESYANE TVCWVHVGDS |
| | 260 270 280 290 300 |
| | AAQTQLKCAR MPGLKGFVDE HTINISLSLH HVEQMAIDWL TGNFYFVDDI |
| | 310 320 330 340 350 |
| | DDRIFVCNRN GDTCVILLDL ELYNPKGIAL DPAMGKVFFT DYGQIPKVER |
| | 360 370 380 390 400 |
| | CDMDGQNRTK LVDSKIVEPH GITLDLVSRL VYWADAYLDY IEVVDYEGKG |
| | 410 420 430 440 450 |
| | RQTIIQGILI EHLYGLTVFE NYLYATNSDN ANAQQKTSVI RVNRENSTEY |
| | 460 470 480 490 500 |
| | QVVTRVDKGG ALHIYHQRRQ PRVRSHACEN DQYGKPGGCS DICLLANSHK |
| | 510 520 530 540 550 |
| | ARTCRCRSGF SLGSDGKSCK KPEHELFLVY GKGRPGIIRG MDMGAKVPDE |
| | 560 570 580 590 600 |
| | HMIPIENLMN PRALDFHAET GFIYFADTTS YLIGRQKIDG TERETILKDG |
| | 610 620 630 640 650 |
| | IHNVEGVAVD WMGDNLYWTD DGPKKTISVA RLEKAAQTRK TLIEGKMTHP |
| | 660 670 680 690 700 |
| | RAIVVDPING WMYWTDWEED PKDSRRGRLE RAWMDGSHRD IFVTSKTVLW |
| | 710 720 730 740 750 |
| | PNGLSLDIPA GRLYWVDAFY DRIETILLNG TDRKIVYEGP ELNHAFGICH |
| | 760 770 780 790 800 |
| | HGNYLFWTEY RSGSVYRLER GVGGAPPTVT LLRSERPPIF EIRMYDAQQQ |
| | 810 820 830 840 850 |
| | QVGINKCRVN NGGCSSLCLA TPGSRQCACA EDQVIDADGV TCLANPSYVP |
| | 860 870 880 890 900 |
| | PPQCQPGEFA CANSRCIQER WKCDGDNDCL DNSDEAPALC HQHTCPSDRF |
| | 910 920 930 940 950 |
| | KCENNRCIPN RWLCDGDNDC GNSEDESNAT CSARTCPPNQ FSCASGRCIP |
| | 960 970 980 990 1000 |
| | ISWTCDLDDD CGDRSDESAS CAYPTCFPLT QFTCNNGRCI NINWRCDNDN |
| | 1010 1020 1030 1040 1050 |
| | DCGDNSDEAG CSHSCSSTQF KCNSGRCIPE HWTCDGDNDC GDYSDETHAN |
| | 1060 1070 1080 1090 1100 |
| | CINQATRPPG GCHTDEFQCR LDGLCIPLRW RCDGDTDCMD SSDEKSCEGV |
| | 1110 1120 1130 1140 1150 |
| | THVCDPSVKF GCKDSARCIS KAWVCDGDND CEDNSDEENC ESLACRPPSH |
| | 1160 1170 1180 1190 1200 |
| | PCANNTSVCL PPDKLCDGND DCGDGSDEGE LCDQCSINNG GCSHNCSVAP |
| | 1210 1220 1230 1240 1250 |
| | GEGIVCSCPL GMELGPDNHT CQIQSYCAKH LKCSQKCDQN KFSVKCSCYE |
| | 1260 1270 1280 1290 1300 |
| | GWVLEPDGES CRSLDPFKPF IIFSNRHEIR RIDLHKGDYS VLVPGLRNTI |
| | 1310 1320 1330 1340 1350 |
| | ALDFHLSQSA LYWTDVVEDK IYRGKLIDNG ALTSFEVVIQ YGLATPEGLA |
| | 1360 1370 1380 1390 1400 |
| | VDWIAGNIYW VESNLDQIEV AKLDGTLRTT LLAGDIEHPR AIALDPRDGI |
| | 1410 1420 1430 1440 1450 |
| | LFWTDWDASL PRIEAASMSG AGRRTVHRET GSGGWPNGLT VDYLEKRILW |
| | 1460 1470 1480 1490 1500 |
| | IDARSDAIYS ARYDGSGHME VIRGHEFLSH PFAVTLYGGE VYWTDWRINT |
| | 1510 1520 1530 1540 1550 |
| | LAKANKWTGH NVTVVQRTNT QPFDLQVYHP SRQPMAPNPC EANGGQGPCS |
| | 1560 1570 1580 1590 1600 |
| | HICLINYNRT VSCACPHLMK LHKDNTTCYE FKKELLYARQ MEIRGVDLDA |
| | 1610 1620 1630 1640 1650 |
| | PYYNYIISFT VPDIDNVTVL DYDAREQRVY WSDVRTQAIK RAFINGTGVE |
| | 1660 1670 1680 1690 1700 |
| | TVVSADLPNA HGLAVDWVSR NLFWTSYDTN KKQINVARLD GSFKNAVVQG |
| | 1710 1720 1730 1740 1750 |
| | LEQPHGLVVH PLRGKLYWTD GDNISMANMD GSNRTLLESG QKGPVGLAID |
| | 1760 1770 1780 1790 1800 |
| | FPESKLYWIS SGNHTINRCN LDGSGLEVID AMRSQLGKAT ALAIMGDKLW |
| | 1810 1820 1830 1840 1850 |
| | WADQVSEKMG TCSKADGSGS VVLRNSTTLV MHMKVYDESI QLDHKGTNPC |
| | 1860 1870 1880 1890 1900 |
| | SVNNGDCSQL CLPTSETTRS CMCTAGYSLR SGQQACEGVG SFLLYSVHEG |
| | 1910 1920 1930 1940 1950 |
| | IRGIPLDPND KSDALVPVSG TSLAVGIDFH AENDTIYWVD MGLSTISRAK |
| | 1960 1970 1980 1990 2000 |
| | RDQTWREDVV TNGIGRVEGI AVDWIAGNIY WTDQGEDVIE VARINGSFRY |
| | 2010 2020 2030 2040 2050 |
| | VVISQGLDKP RAITVHPEKG YLFWTEWGQY PRIERSRLDG TERVVLVNVS |
| | 2060 2070 2080 2090 2100 |
| | ISWPNGISVD YQDGKLYWCD ARTDKIERID LETGENREVV LSSNNMDMES |
| | 2110 2120 2130 2140 2150 |
| | VSVFEDFIYW SDRTHANGSI KRGSKDNATD SVPLRTGIGV QLKDIKVENR |
| | 2160 2170 2180 2190 2200 |
| | DRQKGTNVCA VANGGCQQLC LYRGRGQRAC ACAHGMLAED GASCREYAGY |
| | 2210 2220 2230 2240 2250 |
| | LLYSERTILK SIHLSDERNL NAPVQPFEDP EHMKNVIALA FDYRAGTSPG |
| | 2260 2270 2280 2290 2300 |
| | TPNRIFFSDI HEGNIQQIND DGSRRITIVE NVGSVEGLAY HRGWDTLYWT |
| | 2310 2320 2330 2340 2350 |
| | SYTTSTITRH TVDQTRPGAF ERETVITMSG DDHPRAFVLD ECQNIMFWIN |
| | 2360 2370 2380 2390 2400 |
| | WNEQHPSIMR AALSGANVLT LIEKDIRTPN GLAIDHRAEK LYFSDATLDK |
| | 2410 2420 2430 2440 2450 |
| | IERCEYDGSH RYVILKSEPV HPFGLAVYGE HIFWTDWVRR AVQRANKHVG |
| | 2460 2470 2480 2490 2500 |
| | SNMKLLRVDI PQQPMGIIAV ANDINSCELS PCRINNGGCQ DLCLLTHQGH |
| | 2510 2520 2530 2540 2550 |
| | VNCSCRGGRI LQDDLTCRAV NSSCRAQDEF ECANGECINF SLTCDGVPHC |
| | 2560 2570 2580 2590 2600 |
| | KDKSDEKPSY CNSRRCKKTF RQCSNGRCVS NMLWCNGADD CGDGSDEIPC |
| | 2610 2620 2630 2640 2650 |
| | NKTACGVGEF RCRDGTCIGN SSRCNQFVDC EDASDEMNCS ATDCSSYFRL |
| | 2660 2670 2680 2690 2700 |
| | GVKGVLFQPC ERTSLCYAPS WVCDGANDCG DYSDERDCPG VKRPRCPLNY |
| | 2710 2720 2730 2740 2750 |
| | FACPSGRCIP MSWTCDKEDD CEHGEDETHC NKFCSEAQFE CQNHRCISKQ |
| | 2760 2770 2780 2790 2800 |
| | WLCDGSDDCG DGSDEAAHCE GKTCGPSSFS CPGTHVCVPE RWLCDGDKDC |
| | 2810 2820 2830 2840 2850 |
| | ADGADESIAA GCLYNSTCDD REEMCQNRQC IPKHFVCDHD RDCADGSDES |
| | 2860 2870 2880 2890 2900 |
| | PECEYPTCGP SEFRCANGRC LSSRQWECDG ENDCHDQSDE APKNPHCTSQ |
| | 2910 2920 2930 2940 2950 |
| | EHKCNASSQF LCSSGRCVAE ALLQNGQDDC GDSSDERGCH INECISRKLS |
| | 2960 2970 2980 2990 3000 |
| | GCSQDCEDLK IGFKCRCRPG FRLKDDGRTC ADVDECSTTE PCSQRCINTH |
| | 3010 3020 3030 3040 3050 |
| | GSYKCLCVEG YAPRGGDPHS CKAVTDEEPF LIFANRYYLR KLNLDGSNYT |
| | 3060 3070 3080 3090 3100 |
| | LLKQGLNNAV ALDFDYREQM IYWTDVTTQG SMIRRMHLNG SNVQVLHRTG |
| | 3110 3120 3130 3140 3150 |
| | LSNPDGLAVD WVGGNLYWCD KGRDTIEVSK LNGAYRTVLV SSGLREPRAL |
| | 3160 3170 3180 3190 3200 |
| | VVDVQNGYLY WTDWGDHSLI GRIGMDGSSR SVIVDTKITW PNGLTLDYVT |
| | 3210 3220 3230 3240 3250 |
| | ERIYWADARE DYIEFASLDG SNRHVVLSQD IPHIFALTLF EDYVYWTDWE |
| | 3260 3270 3280 3290 3300 |
| | TKSINRAHKT TGINKTLLIS TLHRPMDLHV FHALRQPDVP NHPCKVNNGG |
| | 3310 3320 3330 3340 3350 |
| | CSNICLLSPG GGHKCACPTN FYLGSDGRTC VSNCTASQFV CKNDKCIPEW |
| | 3360 3370 3380 3390 3400 |
| | WKCDTEDDCG DHSDEPPDCP EFKCRPGQFQ CSTGICTNPA FICDGDNDCQ |
| | 3410 3420 3430 3440 3450 |
| | DNSDEANCDI HVCLPSQFKC TNTNRCIPGI FRQNGQDNCG DGEDERDCPE |
| | 3460 3470 3480 3490 3500 |
| | VTCAPNQFQC SITKRCIPRV WVCDRDNDCV DGSDEPANCT QMTCGVDEFR |
| | 3510 3520 3530 3540 3550 |
| | CKDSGRCIPA RWKCDGEDDC GDGSDEPKEE CDERTCEPYQ FRCKNNRCVP |
| | 3560 3570 3580 3590 3600 |
| | GRWQCDYDND CGDNSDEESC TPRPCSESEF SCANGRCIAG RWKCDGDHDC |
| | 3610 3620 3630 3640 3650 |
| | ADGSDEKDCT PRCDMDQFQC KSGHCIPLRW RCDADADCMD GSDEEACGTG |
| | 3660 3670 3680 3690 3700 |
| | VRTCPLDEFQ CNNTLCKPLA WKCDGEDDCG DNSDENPEEC ARFVCPPNRP |
| | 3710 3720 3730 3740 3750 |
| | FRCKNDRVCL WIGRQCDGTD NCGDGTDEED CEPPTAHTTH CKDKKEFLCR |
| | 3760 3770 3780 3790 3800 |
| | NQRCLSSSLR CNMEDDCGDG SDEEDCSIDP KLISCATNAS ICGDEARCVR |
| | 3810 3820 3830 3840 3850 |
| | TEKAAYCACR SGFHTVPGQP GCQDINECLR FGTCSQLCNN TKGGHLCSCA |
| | 3860 3870 3880 3890 3900 |
| | RNFMKTHNTC KAEGSEYQVL YIADDNEIRS LFPGHPHSAY EQAFQGDESV |
| | 3910 3920 3930 3940 3950 |
| | RIDAMDVHVK AGRVYWINWH TGTISYRSLP PAAPPTTSNR HRRQIDRGVT |
| | 3960 3970 3980 3990 4000 |
| | HLNISGLKMP RGIAIDWVAG NVYWTDSGRD VIEVAQMKGE NRKTLISGMI |
| | 4010 4020 4030 4040 4050 |
| | DEPHAIVVDP LRGTMYWSDW GNHPKIETAA MDGTLRETLV QDNIQWPTGL |
| | 4060 4070 4080 4090 4100 |
| | AVDYHNERLY WADAKLSVIG SIRLNGTDPI VAADSKRGLS HPFSIDVFED |
| | 4110 4120 4130 4140 4150 |
| | YIYGVTYINN RVFKIHKFGH SPLVNLTGGL SHASDVVLYH QHKQPEVTNP |
| | 4160 4170 4180 4190 4200 |
| | CDRKKCEWLC LLSPSGPVCT CPNGKRLDNG TCVPVPSPTP PPDAPRPGTC |
| | 4210 4220 4230 4240 4250 |
| | NLQCENGGSC FLNARRQPKC RCQPRYTGDK CELDQCWEHC RNGGTCAASP |
| | 4260 4270 4280 4290 4300 |
| | SGMPTCRCPT GFTGPKCTQQ VCAGYCANNS TCTVNQGNQP QCRCLPGELG |
| | 4310 4320 4330 4340 4350 |
| | DRCQYRQCSG YCENEGTCQM AADGSRQCRC TAYFEGSRCE VNKCSRCLEG |
| | 4360 4370 4380 4390 4400 |
| | ACVVNKQSGD VTCNCTDGRV APSCLTCVGH CSNGGSCTMN SKMMPECQCP |
| | 4410 4420 4430 4440 4450 |
| | PHMTGPRCEE HVESQQQPGH IASILIPLLL LLLLVIVAGV VEWYKRRVQG |
| | 4460 4470 4480 4490 4500 |
| | AKGFQHQRMT NGAMNVEIGN PTYKMYEGGE PDDVGGLLDA DFALDPDKPT |
| | 4510 4520 4530 4540 |
| | NETNPVYATL YMGGHGSRHS LASTDEKREL LGRGPEDEIG DPLA |
| MAP6 | Microtubule-associated protein 6 (SEQ ID NO: 50) |
| Uniprot | 10 20 30 40 50 |
| Q96JE9 | MAWPCITRAC CIARFWNQLD KADIAVPLVF TKYSEATEHP GAPPQPPPPQ |
| | 60 70 80 90 100 |
| | QQAQPALAPP SARAVAIETQ PAQGELDAVA RATGPAPGPT GEREPAAGPG |
| | 110 120 130 140 150 |
| | RSGPGPGIGS GSTSGPADSV MRQDYRAWKV QRPEPSCRPR SEYQPSDAPE |
| | 160 170 180 190 200 |
| | ERETQYQKDF RAWPLPRRGD HPWIPKPVQI SAASQASAPI LGAPKRRPQS |
| | 210 220 230 240 250 |
| | QERWPVQAAA EAREQEAAPG GAGGLAAGKA SGADERDTRR KAGPAWIVRR |
| | 260 270 280 290 300 |
| | AEGLGHEQTP LPAAQAQVQA TGPEAGRGRA AADALNRQIR EEVASAVSSS |
| | 310 320 330 340 350 |
| | YRNEFRAWTD IKPVKPIKAK PQYKPPDDKM VHETSYSAQF KGEASKPTTA |
| | 360 370 380 390 400 |
| | DNKVIDRRRI RSLYSEPFKE PPKVEKPSVQ SSKPKKTSAS HKPTRKAKDK |
| | 410 420 430 440 450 |
| | QAVSGQAAKK KSAEGPSTTK PDDKEQSKEM NNKLAEAKES LAQPVSDSSK |
| | 460 470 480 490 500 |
| | TQGPVATEPD KDQGSVVPGL LKGQGPMVQE PLKKQGSVVP GPPKDLGPMI |
| | 510 520 530 540 550 |
| | PLPVKDQDHT VPEPLKNESP VISAPVKDQG PSVPVPPKNQ SPMVPAKVKD |
| | 560 570 580 590 600 |
| | QGSVVPESLK DQGPRIPEPV KNQAPMVPAP VKDEGPMVSA SVKDQGPMVS |
| | 610 620 630 640 650 |
| | APVKDQGPIV PAPVKGEGPI VPAPVKDEGP MVSAPIKDQD PMVPEHPKDE |
| | 660 670 680 690 700 |
| | SAMATAPIKN QGSMVSEPVK NQGLVVSGPV KDQDVVVPEH AKVHDSAVVA |
| | 710 720 730 740 750 |
| | PVKNQGPVVP ESVKNQDPIL PVLVKDQGET VLQPPKNQGR IVPEPLKNQV |
| | 760 770 780 790 800 |
| | PIVPVPLKDQ DPLVPVPAKD QGPAVPEPLK TQGPRDPQLP TVSPLPRVMI |
| | 810 |
| | PTAPHTEYIE SSP |
| MB | Myoglobin (SEQ ID NO: 51) |
| Uniprot | 10 20 30 40 50 |
| P02144 | MGLSDGEWQL VLNVWGKVEA DIPGHGQEVL IRLFKGHPET LEKEDKFKHL |
| | 60 70 80 90 100 |
| | KSEDEMKASE DLKKHGATVL TALGGILKKK GHHEAEIKPL AQSHATKHKI |
| | 110 120 130 140 150 |
| | PVKYLEFISE CIIQVLQSKH PGDFGADAQG AMNKALELFR KDMASNYKEL |
| | GFQG |
| MGATI | Alpha-1,3-mannosyl-glycoprotein 2-beta-N-acetylglucosaminyltransferase |
| Uniprot | (SEQ ID NO: 52) |
| P26572 | 10 20 30 40 50 |
| | MLKKQSAGLV LWGAILFVAW NALLLLFFWT RPAPGRPPSV SALDGDPASL |
| | 60 70 80 90 100 |
| | TREVIRLAQD AEVELERQRG LLQQIGDALS SQRGRVPTAA PPAQPRVPVT |
| | 110 120 130 140 150 |
| | PAPAVIPILV IACDRSTVRR CLDKLLHYRP SAELFPIIVS QDCGHEETAQ |
| | 160 170 180 190 200 |
| | AIASYGSAVT HIRQPDLSSI AVPPDHRKFQ GYYKIARHYR WALGQVFRQE |
| | 210 220 230 240 250 |
| | RFPAAVVVED DLEVAPDFFE YFRATYPLLK ADPSLWCVSA WNDNGKEQMV |
| | 260 270 280 290 300 |
| | DASRPELLYR TDEFPGLGWL LLAELWAELE PKWPKAFWDD WMRRPEQRQG |
| | 310 320 330 340 350 |
| | RACIRPEISR TMTEGRKGVS HGQFFDQHLK FIKLNQQFVH FTQLDLSYLQ |
| | 360 370 380 390 400 |
| | REAYDRDELA RVYGAPQLQV EKVRTNDRKE LGEVRVQYTG RDSFKAFAKA |
| | 410 420 430 440 |
| | LGVMDDLKSG VPRAGYRGIV TFQFRGRRVH LAPPLTWEGY DPSWN |
| MYO1B | Unconventional myosin-Ib (SEQ ID NO: 53) |
| Uniprot | 10 20 30 40 50 |
| O43795 | MAKMEVKTSL LDNMIGVGDM VLLEPINEET FINNLKKRED HSEIYTYIGS |
| | 60 70 80 90 100 |
| | VVISVNPYRS LPIYSPEKVE EYRNRNFYEL SPHIFALSDE AYRSLRDQDK |
| | 110 120 130 140 150 |
| | DQCILITGES GAGKTEASKL VMSYVAAVCG KGAEVNQVKE QLLQSNPVLE |
| | 160 170 180 190 200 |
| | AFGNAKTVRN DNSSRFGKYM DIEFDEKGDP LGGVISNYLL EKSRVVKQPR |
| | 210 220 230 240 250 |
| | GERNFHVFYQ LLSGASEELL NKLKLERDES RYNYLSLDSA KVNGVDDAAN |
| | 260 270 280 290 300 |
| | FRTVRNAMQI VGEMDHEAES VLAVVAAVLK LGNIEFKPES RVNGLDESKI |
| | 310 320 330 340 350 |
| | KDKNELKEIC ELTGIDQSVL ERAFSFRTVE AKQEKVSTTL NVAQAYYARD |
| | 360 370 380 390 400 |
| | ALAKNLYSRL FSWLVNRINE SIKAQTKVRK KVMGVLDIYG FEIFEDNSFE |
| | 410 420 430 440 450 |
| | QFIINYCNEK LQQIFIELTL KEEQEEYIRE DIEWTHIDYF NNAIICDLIE |
| | 460 470 480 490 500 |
| | NNINGILAML DEECLRPGTV TDETELEKLN QVCATHQHFE SRMSKCSREL |
| | 510 520 530 540 550 |
| | NDTSLPHSCF RIQHYAGKVL YQVEGFVDKN NDLLYRDLSQ AMWKASHALI |
| | 560 570 580 590 600 |
| | KSLFPEGNPA KINLKRPPTA GSQFKASVAT LMKNLQTKNP NYIRCIKPND |
| | 610 620 630 640 650 |
| | KKAAHIFNEA LVCHQIRYLG LLENVRVRRA GYAFRQAYEP CLERYKMLCK |
| | 660 670 680 690 700 |
| | QTWPHWKGPA RSGVEVLENE LEIPVEEYSF GRSKIFIRNP RTLFKLEDLR |
| | 710 720 730 740 750 |
| | KQRLEDLATL IQKIYRGWKC RTHFLIMKKS QIVIAAWYRR YAQQKRYQQT |
| | 760 770 780 790 800 |
| | KSSALVIQSY IRGWKARKIL RELKHQKRCK EAVTTIAAYW HGTQARRELR |
| | 810 820 830 840 850 |
| | RIKEEARNKH AIAVIWAYWL GSKARRELKR LKEEARRKHA VAVIWAYWLG |
| | 860 870 880 890 900 |
| | LKVRREYRKE FRANAGKKIY EFTLQRIVQK YFLEMKNKMP SLSPIDKNWP |
| | 910 920 930 940 950 |
| | SRPYLFLDST HKELKRIFHL WRCKKYRDQF TDQQKLIYEE KLEASELFKD |
| | 960 970 980 990 1000 |
| | KKALYPSSVG QPFQGAYLEI NKNPKYKKLK DAIEEKIIIA EVVNKINRAN |
| | 1010 1020 1030 1040 1050 |
| | GKSTSRIFLL TNNNLLLADQ KSGQIKSEVP LVDVTKVSMS SQNDGFFAVH |
| | 1060 1070 1080 1090 1100 |
| | LKEGSEAASK GDFLESSDHL IEMATKLYRT TLSQTKQKLN IEISDEFLVQ |
| | 1110 1120 1130 |
| | FRQDKVCVKF IQGNQKNGSV PTCKRKNNRL LEVAVP |
| NAB2 | NGFI-A-binding protein 2 (SEQ ID NO: 54) |
| Uniprot | 10 20 30 40 50 |
| Q15742 | MHRAPSPTAE QPPGGGDSAR RTLQPRLKPS ARAMALPRTL GELQLYRVLQ |
| | 60 70 80 90 100 |
| | RANLLSYYET FIQQGGDDVQ QLCEAGEEEF LEIMALVGMA TKPLHVRRLQ |
| | 110 120 130 140 150 |
| | KALREWATNP GLFSQPVPAV PVSSIPLFKI SETAGTRKGS MSNGHGSPGE |
| | 160 170 180 190 200 |
| | KAGSARSFSP KSPLELGEKL SPLPGGPGAG DPRIWPGRST PESDVGAGGE |
| | 210 220 230 240 250 |
| | EEAGSPPESP PAGGGVPEGT GAGGLAAGGT GGGPDRLEPE MVRMVVESVE |
| | 260 270 280 290 300 |
| | RIFRSFPRGD AGEVTSLLKL NKKLARSVGH IFEMDDNDSQ KEEEIRKYSI |
| | 310 320 330 340 350 |
| | IYGREDSKRR EGKQLSLHEL TINEAAAQFC MRDNTLLLRR VELFSLSRQV |
| | 360 370 380 390 400 |
| | ARESTYLSSL KGSRLHPEEL GGPPLKKLKQ EVGEQSHPEI QQPPPGPESY |
| | 410 420 430 440 450 |
| | VPPYRPSLEE DSASLSGESL DGHLQAVGSC PRLTPPPADL PLALPAHGLW |
| | 460 470 480 490 500 |
| | SRHILQQTLM DEGLRLARLV SHDRVGRLSP CVPAKPPLAE FEEGLLDRCP |
| | 560 570 |
| | APGPHPALVE GRRSSVKVEA EASRQ |
| PCDH1 | Protocadherin-1 (SEQ ID NO: 55) |
| Uniprot | 10 20 30 40 50 |
| Q08174 | MDSGAGGRRC PEAALLILGP PRMEHLRHSP GPGGQRILLP SMLLALLLLL |
| | 60 70 80 90 100 |
| | APSPGHATRV VYKVPEEQPP NTLIGSLAAD YGEPDVGHLY KLEVGAPYLR |
| | 110 120 130 140 150 |
| | VDGKTGDIFT TETSIDREGL RECQNQLPGD PCILEFEVSI TDLVQNGSPR |
| | 160 170 180 190 200 |
| | LLEGQIEVQD INDNTPNEAS PVITLAIPEN TNIGSLFPIP LASDRDAGPN |
| | 210 220 230 240 250 |
| | GVASYELQAG PEAQELFGLQ VAEDQEEKQP QLIVMGNLDR ERWDSYDLTI |
| | 260 270 280 290 300 |
| | KVQDGGSPPR ASSALLRVTV LDINDNAPKF ERPSYEAELS ENSPIGHSVI |
| | 310 320 330 340 350 |
| | QVKANDSDQG ANAEIEYTFH QAPEVVRRLL RLDRNTGLIT VQGPVDREDL |
| | 360 370 380 390 400 |
| | STLRESVLAK DRGTNPKSAR AQVVVTVKDM NDNAPTIEIR GIGLVTHQDG |
| | 410 420 430 440 450 |
| | MANISEDVAE ETAVALVQVS DRDEGENAAV TCVVAGDVPE QLRQASETGS |
| | 460 470 480 490 500 |
| | DSKKKYFLQT TTPLDYEKVK DYTIEIVAVD SGNPPLSSTN SLKVQVVDVN |
| | 560 570 580 590 600 |
| | DNAPVFTQSV TEVAFPENNK PGEVIAEITA SDADSGSNAE LVYSLEPEPA |
| | 560 570 580 590 600 |
| | AKGLFTISPE TGEIQVKTSL DREQRESYEL KVVAADRGSP SLQGTATVLV |
| | 610 620 630 640 650 |
| | NVLDCNDNDP KEMLSGYNFS VMENMPALSP VGMVIVIDGD KGENAQVQLS |
| | 660 670 680 690 700 |
| | VEQDNGDFVI QNGTGTILSS LSFDREQQST YTFQLKAVDG GVPPRSAYVG |
| | 710 720 730 740 750 |
| | VTINVLDEND NAPYITAPSN TSHKLLTPQT RLGETVSQVA AEDEDSGVNA |
| | 760 770 780 790 800 |
| | ELIYSIAGGN PYGLFQIGSH SGAITLEKEI ERRHHGLHRL VVKVSDRGKP |
| | 810 820 830 840 850 |
| | PRYGTALVHL YVNETLANRT LLETLLGHSL DTPLDIDIAG DPEYERSKQR |
| | 860 870 880 890 900 |
| | GNILFGVVAG VVAVALLIAL AVLVRYCRQR EAKSGYQAGK KETKDLYAPK |
| | 910 920 930 940 950 |
| | PSGKASKGNK SKGKKSKSPK PVKPVEDEDE AGLQKSLKEN IMSDAPGDSP |
| | 960 970 980 990 1000 |
| | RIHLPLNYPP GSPDLGRHYR SNSPLPSIQL QPQSPSASKK HQVVQDLPPA |
| | 1010 1020 1030 1040 1050 |
| | NTFVGTGDTT STGSEQYSDY SYRINPPKYP SKQVGQPFQL STPQPLPHPY |
| | 1060 |
| | HGAIWTEVWE |
| PDLIM1 | PDZ and LIM domain protein 1 (SEQ ID NO: 56) |
| Uniprot | 10 20 30 40 50 |
| O00151 | MTTQQIDLQG PGPWGFRLVG GKDFEQPLAI SRVTPGSKAA LANLCIGDVI |
| | 60 70 80 90 100 |
| | TAIDGENTSN MTHLEAQNRI KGCTDNLTLT VARSEHKVWS PLVTEEGKRH |
| | 110 120 130 140 150 |
| | PYKMNLASEP QEVLHIGSAH NRSAMPFTAS PASSTTARVI TNQYNNPAGL |
| | 160 170 180 190 200 |
| | YSSENISNEN NALESKTAAS GVEANSRPLD HAQPPSSIVI DKESEVYKML |
| | 210 220 230 240 250 |
| | QEKQELNEPP KQSTSELVLQ EILESEEKGD PNKPSGERSV KAPVTKVAAS |
| | 260 270 280 290 300 |
| | IGNAQKLPMC DKCGTGIVGV FVKLRDRHRH PECYVCTDCG TNLKQKGHEF |
| | 310 320 |
| | VEDQIYCEKH ARERVTPPEG YEVVTVEPK |
| PLA2G6 | 85/88 kDa calcium-independent phospholipase A2 (SEQ ID NO: 57) |
| Uniprot | 10 20 30 40 50 |
| O60733 | MQFFGRLVNT FSGVINLESN PERVKEVAVA DYTSSDRVRE EGQLILFQNT |
| | 60 70 80 90 100 |
| | PNRTWDCVLV NPRNSQSGER LEQLELEADA LVNFHQYSSQ LLPFYESSPQ |
| | 110 120 130 140 150 |
| | VLHTEVLQHL TDLIRNHPSW SVAHLAVELG IRECFHHSRI ISCANCAENE |
| | 160 170 180 190 200 |
| | EGCTPLHLAC RKGDGEILVE LVQYCHTQMD VTDYKGETVE HYAVQGDNSQ |
| | 210 220 230 240 250 |
| | VLQLLGRNAV AGLNQVNNQG LTPLHLACQL GKQEMVRVLL LCNARCNIMG |
| | 260 270 280 290 300 |
| | PNGYPIHSAM KFSQKGCAEM IISMDSSQIH SKDPRYGASP LHWAKNAEMA |
| | 310 320 330 340 350 |
| | RMLLKRGCNV NSTSSAGNTA LHVAVMRNRF DCAIVLLTHG ANADARGEHG |
| | 360 370 380 390 400 |
| | NTPLHLAMSK DNVEMIKALI VEGAEVDTPN DFGETPTFLA SKIGRLVTRK |
| | 410 420 430 440 450 |
| | AILTLLRTVG AEYCFPPIHG VPAEQGSAAP HHPFSLERAQ PPPISLNNLE |
| | 460 470 480 490 500 |
| | LQDLMHISRA RKPAFILGSM RDEKRTHDHL LCLDGGGVKG LIIIQLLIAI |
| | 510 520 530 540 550 |
| | EKASGVATKD LEDWVAGTST GGILALAILH SKSMAYMRGM YERMKDEVER |
| | 560 570 580 590 600 |
| | GSRPYESGPL EEFLKREFGE HTKMTDVRKP KVMLIGTLSD RQPAELHLER |
| | 610 620 630 640 650 |
| | NYDAPETVRE PRFNQNVNLR PPAQPSDQLV WRAARSSGAA PTYFRPNGRE |
| | 660 670 680 690 700 |
| | LDGGLLANNP TLDAMTEIHE YNQDLIRKGQ ANKVKKLSIV VSLGTGRSPQ |
| | 710 720 730 740 750 |
| | VPVTCVDVER PSNPWELAKT VEGAKELGKM VVDCCTDPDG RAVDRARAWC |
| | 760 770 780 790 800 |
| | EMVGIQYFRL NPQLGTDIML DEVSDTVLVN ALWETEVYIY EHREEFQKLI |
| | QLLLSP |
| PREX1 | Phosphatidylinositol 3,4,5-trisphosphate-dependent Rac exchanger 1 |
| Uniprot | protein (SEQ ID NO: 58) |
| Q8TCU6 | 10 20 30 40 50 |
| | MEAPSGSEPG GDGAGDCAHP DPRAPGAAAP SSGPGPCAAA RESERQLRLR |
| | 60 70 80 90 100 |
| | LCVINEILGT ERDYVGTLRF LQSAFLHRIR QNVADSVEKG LTEENVKVLE |
| | 110 120 130 140 150 |
| | SNIEDILEVH KDFLAALEYC LHPEPQSQHE LGNVFLKEKD KFCVYEEYCS |
| | 160 170 180 190 200 |
| | NHEKALRLLV ELNKIPTVRA FLLSCMLLGG RKTTDIPLEG YLLSPIQRIC |
| | 210 220 230 240 250 |
| | KYPLLLKELA KRTPGKHPDH PAVQSALQAM KTVCSNINET KRQMEKLEAL |
| | 260 270 280 290 300 |
| | EQLQSHIEGW EGSNLTDICT QLLLQGTLLK ISAGNIQERA FFLEDNLLVY |
| | 310 320 330 340 350 |
| | CKRKSRVTGS KKSTKRTKSI NGSLYIFRGR INTEVMEVEN VEDGTADYHS |
| | 360 370 380 390 400 |
| | NGYTVINGWK IHNTAKNKWF VCMAKTAEEK QKWLDAIIRE REQRESLKLG |
| | 410 420 430 440 450 |
| | MERDAYVMIA EKGEKLYHMM MNKKVNLIKD RRRKLSTVPK CELGNEFVAW |
| | 460 470 480 490 500 |
| | LLEIGEISKT EEGVNLGQAL LENGIIHHVS DKHQFKNEQV MYRFRYDDGT |
| | 510 520 530 540 550 |
| | YKARSELEDI MSKGVRLYCR LHSLYTPVIK DRDYHLKTYK SVLPGSKLVD |
| | 560 570 580 590 600 |
| | WLLAQGDCQT REEAVALGVG LCNNGEMHHV LEKSEFRDES QYFRFHADEE |
| | 610 620 630 640 650 |
| | MEGTSSKNKQ LRNDEKLVEN ILAKRLLILP QEEDYGEDIE EKNKAVVVKS |
| | 660 670 680 690 700 |
| | VQRGSLAEVA GLQVGRKIYS INEDLVELRP FSEVESILNQ SFCSRRPLRL |
| | 710 720 730 740 750 |
| | LVATKAKEII KIPDQPDTLC FQIRGAAPPY VYAVGRGSEA MAAGLCAGQC |
| | 760 770 780 790 800 |
| | ILKVNGSNVM NDGAPEVLEH FQAFRSRREE ALGLYQWIYH THEDAQEARA |
| | 810 820 830 840 850 |
| | SQEASTEDPS GEQAQEEDQA DSAFPLLSLG PRISLCEDSP MVTLTVDNVH |
| | 860 870 880 890 900 |
| | LEHGVVYEYV STAGVRCHVL EKIVEPRGCF GLTAKILEAF AANDSVEVEN |
| | 910 920 930 940 950 |
| | CRRLMALSSA IVTMPFEFFR NICDTKLESI GQRIACYQEF AAQLKSRVSP |
| | 960 970 980 990 1000 |
| | PFKQAPLEPH PLCGLDFCPT NCHINLMEVS YPKTTPSVGR SFSIRFGRKP |
| | 1010 1020 1030 1040 1050 |
| | SLIGLDPEQG HLNPMSYTQH CITTMAAPSW KCLPAAEGDP QGQGLHDGSE |
| | 1060 1070 1080 1090 1100 |
| | GPASGTIGQE DRGLSELLKQ EDREIQDAYL QLFTKLDVAL KEMKQYVTQI |
| | 1110 1120 1130 1140 1150 |
| | NRLLSTITEP TSGGSCDASL AEEASSLPLV SEESEMDRSD HGGIKKVCEK |
| | 1160 1170 1180 1190 1200 |
| | VAREDQEDSG HDTMSYRDSY SECNSNRDSV LSYTSVRSNS SYLGSDEMGS |
| | 1210 1220 1230 1240 1250 |
| | GDELPCDMRI PSDKQDKLHG CLEHLENQVD SINALLKGPV MSRAFEETKH |
| | 1260 1270 1280 1290 1300 |
| | FPMNHSLQEF KQKEECTIRG RSLIQISIQE DPWNLPNSIK TLVDNIQRYV |
| | 1310 1320 1330 1340 1350 |
| | EDGKNQLLLA LLKCTDTELQ LRRDAIFCQA LVAAVCTESK QLLAALGYRY |
| | 1360 1370 1380 1390 1400 |
| | NNNGEYEESS RDASRKWLEQ VAATGVLLHC QSLLSPATVK EERTMLEDIW |
| | 1410 1420 1430 1440 1450 |
| | VTLSELDNVT FSFKQLDENY VANTNVFYHI EGSRQALKVI FYLDSYHFSK |
| | 1460 1470 1480 1490 1500 |
| | LPSRLEGGAS LRLHTALFTK VLENVEGLPS PGSQAAEDLQ QDINAQSLEK |
| | 1510 1520 1530 1540 1550 |
| | VQQYYRKIRA FYLERSNLPT DASTTAVKID QLIRPINALD ELCRIMKSFV |
| | 1560 1570 1580 1590 1600 |
| | HPKPGAAGSV GAGLIPISSE LCYRIGACQM VMCGTGMQRS TLSVSLEQAA |
| | 1610 1620 1630 1640 1650 |
| | ILARSHGLLP KCIMQATDIM RKQGPRVEIL AKNLRVKDQM PQGAPRLYRL |
| | CQPPVDGDL |
| PRPF40B | Pre-mRNA-processing factor 40 homolog B (SEQ ID NO: 59) |
| Uniprot | 10 20 30 40 50 |
| Q6NWY9 | MMPPPFMPPP GIPPPFPPMG LPPMSQRPPA IPPMPPGILP PMLPPMGAPP |
| | 60 70 80 90 100 |
| | PLTQIPGMVP PMMPGMLMPA VPVTAATAPG ADTASSAVAG TGPPRALWSE |
| | 110 120 130 140 150 |
| | HVAPDGRIYY YNADDKQSVW EKPSVLKSKA ELLLSQCPWK EYKSDTGKPY |
| | 160 170 180 190 200 |
| | YYNNQSKESR WTRPKDLDDL EVLVKQEAAG KQQQQLPQTL QPQPPQPQPD |
| | 210 220 230 240 250 |
| | PPPVPPGPTP VPTGLLEPEP GGSEDCDVLE ATQPLEQGFL QQLEEGPSSS |
| | 260 270 280 290 300 |
| | GQHQPQQEEE ESKPEPERSG LSWSNREKAK QAFKELLRDK AVPSNASWEQ |
| | 310 320 330 340 350 |
| | AMKMVVTDPR YSALPKLSEK KQAFNAYKAQ REKEEKEEAR LRAKEAKQTL |
| | 360 370 380 390 400 |
| | QHFLEQHERM TSTTRYRRAE QTFGELEVWA VVPERDRKEV YDDVLFFLAK |
| | 410 420 430 440 450 |
| | KEKEQAKQLR RRNIQALKSI LDGMSSVNFQ TTWSQAQQYL MDNPSFAQDH |
| | 460 470 480 490 500 |
| | QLQNMDKEDA LICFEEHIRA LEREEEEERE RARLRERRQQ RKNREAFQTE |
| | 510 520 530 540 550 |
| | LDELHETGQL HSMSTWMELY PAVSTDVREA NMLGQPGSTP LDLEKFYVEE |
| | 560 570 580 590 600 |
| | LKARFHDEKK IIKDILKDRG FCVEVNTAFE DFAHVISEDK RAAALDAGNI |
| | 610 620 630 640 650 |
| | KLTENSLLEK AEAREREREK EEARRMRRRE AAFRSMLRQA VPALELGTAW |
| | 660 670 680 690 700 |
| | EEVREREVCD SAFEQITLES ERIRLFREFL QVLEQTECQH LHTKGRKHGR |
| | 710 720 730 740 750 |
| | KGKKHHHKRS HSPSGSESEE EELPPPSLRP PKRRRRNPSE SGSEPSSSLD |
| | 760 770 780 790 800 |
| | SVESGGAALG GRGSPSSHLL GADHGLRKAK KPKKKTKKRR HKSNSPESET |
| | 810 820 830 840 850 |
| | DPEEKAGKES DEKEQEQDKD RELQQAELPN RSPGFGIKKE KTGWDTSESE |
| | 860 870 |
| | LSEGELERRR RTLLQQLDDH Q |
| RABGGTA | Geranylgeranyl transferase type-2 subunit alpha (SEQ ID NO: 60) |
| Uniprot | 10 20 30 40 50 |
| Q92696 | MHGRLKVKTS EEQAEAKRLE REQKLKLYQS ATQAVFQKRQ AGELDESVLE |
| | 60 70 80 90 100 |
| | LTSQILGANP DFATIWNCRR EVLQQLETQK SPEELAALVK AELGFLESCL |
| | 110 120 130 140 150 |
| | RVNPKSYGTW HHRCWLLGRL PEPNWTRELE LCARFLEVDE RNFHCWDYRR |
| | 160 170 180 190 200 |
| | FVATQAAVPP AEELAFTDSL ITRNESNYSS WHYRSCLLPQ LHPQPDSGPQ |
| | 210 220 230 240 250 |
| | GRIPEDVLLK ELELVQNAFF TDPNDQSAWE YHRWLLGRAD PQDALRCLHV |
| | 260 270 280 290 300 |
| | SRDEACLTVS FSRPLLVGSR MEILLLMVDD SPLIVEWRTP DGRNRPSHVW |
| | 310 320 330 340 350 |
| | LCDLPAASIN DQLPQHTERV IWTAGDVQKE CVLLKGRQEG WQRDSTTDEQ |
| | 360 370 380 390 400 |
| | LERCELSVEK STVLQSELES CKELQELEPE NKWCLLTIIL LMRALDPLLY |
| | 410 420 430 440 450 |
| | EKETLQYFQT LKAVDPMRAT YLDDLRSKEL LENSVLKMEY AEVRVLHLAH |
| | 460 470 480 490 500 |
| | KDLTVICHLE QLLLVTHLDL SHNRLRTLPP ALAALRCLEV LQASDNAIES |
| | 510 520 530 540 550 |
| | LDGVTNLPRL QELLLCNNRL QQPAVLQPLA SCPRIVLLNL QGNPLCQAVG |
| | 560 |
| | ILEQLAELLP SVSSVLT |
| S100A6 | Protein S100-A6 (SEQ ID NO: 61) |
| Uniprot | 10 20 30 40 50 |
| P06703 | MACPLDQAIG LLVAIFHKYS GREGDKHTLS KKELKELIQK ELTIGSKLQD |
| | 60 70 80 90 |
| | AEIARLMEDL DRNKDQEVNE QEYVTELGAL ALIYNEALKG |
| SCNNIA | Amiloride-sensitive sodium channel subunit alpha (SEQ ID NO: 62) |
| Uniprot | 10 20 30 40 50 |
| P37088 | MEGNKLEEQD SSPPQSTPGL MKGNKREEQG LGPEPAAPQQ PTAEEEALIE |
| | 60 70 80 90 100 |
| | FHRSYREIFE FFCNNTTIHG AIRLVCSQHN RMKTAFWAVL WLCTEGMMYW |
| | 110 120 130 140 150 |
| | QFGLLFGEYF SYPVSLNINL NSDKLVFPAV TICTLNPYRY PEIKEELEEL |
| | 160 170 180 190 200 |
| | DRITEQTLED LYKYSSFTTL VAGSRSRRDL RGTLPHPLQR LRVPPPPHGA |
| | 210 220 230 240 250 |
| | RRARSVASSL RDNNPQVDWK DWKIGFQLCN QNKSDCFYQT YSSGVDAVRE |
| | 260 270 280 290 300 |
| | WYRFHYINIL SRLPETLPSL EEDTLGNFIF ACRENQVSCN QANYSHFHHP |
| | 310 320 330 340 350 |
| | MYGNCYTEND KNNSNLWMSS MPGINNGLSL MLRAEQNDFI PLLSTVTGAR |
| | 360 370 380 390 400 |
| | VMVHGQDEPA FMDDGGENLR PGVETSISMR KETLDRLGGD YGDCTKNGSD |
| | 410 420 430 440 450 |
| | VPVENLYPSK YTQQVCIHSC FQESMIKECG CAYIFYPRPQ NVEYCDYRKH |
| | 460 470 480 490 500 |
| | SSWGYCYYKL QVDESSDHLG CFTKCRKPCS VTSYQLSAGY SRWPSVTSQE |
| | 510 520 530 540 550 |
| | WVFQMLSRQN NYTVNNKRNG VAKVNIFFKE LNYKINSESP SVTMVTLLSN |
| | 560 570 580 590 600 |
| | LGSQWSIWFG SSVLSVVEMA ELVEDLLVIM FLMLLRRERS RYWSPGRGGR |
| | 610 620 630 640 650 |
| | GAQEVASTLA SSPPSHFCPH PMSLSLSQPG PAPSPALTAP PPAYATLGPR |
| | 660 |
| | PSPGGSAGAS SSTCPLGGP |
| SHC1 | SHC-transforming protein 1 (SEQ ID NO: 63) |
| Uniprot | 10 20 30 40 50 |
| P29353 | MDLLPPKPKY NPLRNESLSS LEEGASGSTP PEELPSPSAS SLGPILPPLP |
| | 60 70 80 90 100 |
| | GDDSPTTICS FFPRMSNLRL ANPAGGRPGS KGEPGRAADD GEGIVGAAMP |
| | 110 120 130 140 150 |
| | DSGPLPLLQD MNKLSGGGGR RTRVEGGQLG GEEWTRHGSF VNKPTRGWLH |
| | 160 170 180 190 200 |
| | PNDKVMGPGV SYLVRYMGCV EVLQSMRALD FNTRTQVTRE AISLVCEAVP |
| | 210 220 230 240 250 |
| | GAKGATRRRK PCSRPLSSIL GRSNLKFAGM PITLTVSTSS LNLMAADCKQ |
| | 260 270 280 290 300 |
| | IIANHHMQSI SFASGGDPDT AEYVAYVAKD PVNQRACHIL ECPEGLAQDV |
| | 310 320 330 340 350 |
| | ISTIGQAFEL RFKQYLRNPP KLVTPHDRMA GEDGSAWDEE EEEPPDHQYY |
| | 360 370 380 390 400 |
| | NDFPGKEPPL GGVVDMRIRE GAAPGAARPT APNAQTPSHL GATLPVGQPV |
| | 410 420 430 440 450 |
| | GGDPEVRKQM PPPPPCPGRE LEDDPSYVNV QNLDKARQAV GGAGPPNPAI |
| | 460 470 480 490 500 |
| | NGSAPRDLED MKPFEDALRV PPPPQSVSMA EQLRGEPWFH GKLSRREAEA |
| | 510 520 530 540 550 |
| | LLQLNGDFLV RESTTTPGQY VLTGLQSGQP KHLLLVDPEG VVRTKDHRFE |
| | 560 570 580 |
| | SVSHLISYHM DNHLPIISAG SELCLQQPVE RKL |
| SHKBP1 | SH3KBP1-binding protein 1 (SEQ ID NO: 64) |
| Uniprot | 10 20 30 40 50 |
| Q8TBC3 | MAAAATAAEG VPSRGPPGEV IHLNVGGKRF STSRQTLTWI PDSFFSSLLS |
| | 60 70 80 90 100 |
| | GRISTLKDET GAIFIDRDPT VFAPILNFLR TKELDPRGVH GSSLLHEAQF |
| | 110 120 130 140 150 |
| | YGLTPLVRRL QLREELDRSS CGNVLENGYL PPPVFPVKRR NRHSLVGPQQ |
| | 160 170 180 190 200 |
| | LGGRPAPVRR SNTMPPNLGN AGLIGRMLDE KTPPSPSGQP EEPGMVRLVC |
| | 210 220 230 240 250 |
| | GHHNWIAVAY TQFLVCYRLK EASGWQLVES SPRLDWPIER LALTARVHGG |
| | 260 270 280 290 300 |
| | ALGEHDKMVA AATGSEILLW ALQAEGGGSE IGVFHLGVPV EALFFVGNQL |
| | 310 320 330 340 350 |
| | IATSHTGRIG VWNAVTKHWQ VQEVQPITSY DAAGSFLLLG CNNGSIYYVD |
| | 360 370 380 390 400 |
| | VQKFPLRMKD NDLLVSELYR DPAEDGVTAL SVYLTPKTSD SGNWIEIAYG |
| | 410 420 430 440 450 |
| | TSSGGVRVIV QHPETVGSGP QLFQTFTVHR SPVTKIMLSE KHLISVCADN |
| | 460 470 480 490 500 |
| | NHVRTWSVTR FRGMISTQPG STPLASFKIL ALESADGHGG CSAGNDIGPY |
| | 510 520 530 540 550 |
| | GERDDQQVFI QKVVPSASQL FVRLSSTGQR VCSVRSVDGS PTTAFTVLEC |
| | 560 570 580 590 600 |
| | EGSRRLGSRP RRYLLTGQAN GSLAMWDLTT AMDGLGQAPA GGLTEQELME |
| | 610 620 630 640 650 |
| | QLEHCELAPP APSAPSWGCL PSPSPRISLT SLHSASSNTS LSGHRGSPSP |
| | 660 670 680 690 700 |
| | PQAEARRRGG GSFVERCQEL VRSGPDLRRP PTPAPWPSSG LGTPLTPPKM |
| | KLNETSE |
| SNPH | Syntaphilin (SEQ ID NO: 65) |
| Uniprot | 10 20 30 40 50 |
| O15079 | MAMSLPGSRR TSAGSRRRTS PPVSVRDAYG TSSLSSSSNS GSYKGSDSSP |
| | 60 70 80 90 100 |
| | TPRRSMKYTL CSDNHGIKPP TPEQYLTPLQ QKEVCIRHLK ARLKDTQDRL |
| | 110 120 130 140 150 |
| | QDRDTEIDDL KTQLSRMQED WIEEECHRVE AQLALKEARK EIKQLKQVID |
| | 160 170 180 190 200 |
| | TVKNNLIDKD KGLQKYFVDI NIQNKKLETL LHSMEVAQNG MAKEDGTGES |
| | 210 220 230 240 250 |
| | AGGSPARSLT RSSTYTKLSD PAVCGDRQPG DPSSGSAEDG ADSGFAAADD |
| | 260 270 280 290 300 |
| | TLSRTDALEA SSLLSSGVDC GTEETSLHSS FGLGPRFPAS NTYEKLLCGM |
| | 310 320 330 340 350 |
| | EAGVQASCMQ ERAIQTDEVQ YQPDLDTILE KVTQAQVCGT DPESGDRCPE |
| | 360 370 380 390 400 |
| | LDAHPSGPRD PNSAVVVTVG DELEAPEPIT RGPTPQRPGA NPNPGQSVSV |
| | 410 420 430 440 450 |
| | VCPMEEEEEA AVAEKEPKSY WSRHYIVDLL AVVVPAVPTV AWLCRSQRRQ |
| | 460 470 480 490 |
| | GQPIYNISSL LRGCCTVALH SIRRISCRSL SQPSPSPAGG GSQL |
| SUSD2 | Sushi domain-containing protein 2 (SEQ ID NO: 66) |
| Uniprot | 10 20 30 40 50 |
| Q9UGT4 | MKPALLPWAL LLLATALGPG PGPTADAQES CSMRCGALDG PCSCHPTCSG |
| | 60 70 80 90 100 |
| | LGTCCLDERD FCLEILPYSG SMMGGKDFVV RHFKMSSPTD ASVICREKDS |
| | 110 120 130 140 150 |
| | IQTLGHVDSS GQVHCVSPLL YESGRIPFTV SLDNGHSEPR AGTWLAVHPN |
| | 160 170 180 190 200 |
| | KVSMMEKSEL VNETRWQYYG TANTSGNLSL TWHVKSLPTQ TITIELWGYE |
| | 210 220 230 240 250 |
| | ETGMPYSQEW TAKWSYLYPL ATHIPNSGSF TETPKPAPPS YQRWRVGALR |
| | 260 270 280 290 300 |
| | IIDSKNYAGQ KDVQALWIND HALAWHLSDD FREDPVAWAR TQCQAWEELE |
| | 310 320 330 340 350 |
| | DQLPNFLEEL PDCPCTLTQA RADSGREFTD YGCDMEQGSV CTYHPGAVHC |
| | 360 370 380 390 400 |
| | VRSVQASIRY GSGQQCCYTA DGTQLLTADS SGGSTPDRGH DWGAPPERTP |
| | 410 420 430 440 450 |
| | PRVPSMSHWL YDVLSFYYCC LWAPDCPRYM QRRPSNDQRN YRPPRLASAF |
| | 460 470 480 490 500 |
| | GDPHEVTEDG TNFTENGRGE YVLLEAALTD LRVQARAQPG TMSNGTETRG |
| | 510 520 530 540 550 |
| | TGLTAVAVQE GNSDVVEVRL ANRIGGLEVL LNQEVLSFTE QSWMDLKGME |
| | 560 570 580 590 600 |
| | LSVAAGDRVS IMLASGAGLE VSVQGPFLSV SVLLPEKELT HTHGLLGTLN |
| | 610 620 630 640 650 |
| | NDPTDDETLH SGRVIPPGTS PQELFLEGAN WTVHNASSLL TYDSWELVAN |
| | 660 670 680 690 700 |
| | FLYQPKHDPT FEPLEPSETT INPSLAQEAA KLCGDDHFCN FDVAATGSLS |
| | 710 720 730 740 750 |
| | TGTATRVAHQ LHQRRMQSLQ PVVSCGWLAP PPNGQKEGNR YLAGSTIYFH |
| | 760 770 780 790 800 |
| | CDNGYSLAGA ETSTCQADGT WSSPTPKCQP GRSYAVLLGI IFGGLAVVAA |
| | 810 820 |
| | VALVYVLLRR RKGNTHVWGA QP |
| THBS1 | Thrombospondin-1 (SEQ ID NO: 67) |
| Uniprot | 10 20 30 40 50 |
| P07996 | MGLAWGLGVL FLMHVCGTNR IPESGGDNSV FDIFELTGAA RKGSGRRLVK |
| | 60 70 80 90 100 |
| | GPDPSSPAFR IEDANLIPPV PDDKFQDLVD AVRAEKGFLL LASLRQMKKT |
| | 110 120 130 140 150 |
| | RGTILALERK DHSGQVFSVV SNGKAGTLDL SLTVQGKQHV VSVEEALLAT |
| | 160 170 180 190 200 |
| | GQWKSITLEV QEDRAQLYID CEKMENAELD VPIQSVFTRD LASIARLRIA |
| | 210 220 230 240 250 |
| | KGGVNDNFQG VLQNVRFVFG TTPEDILRNK GCSSSTSVLL TLDNNVVNGS |
| | 260 270 280 290 300 |
| | SPAIRINYIG HKTKDLQAIC GISCDELSSM VLELRGERTI VTTLQDSIRK |
| | 310 320 330 340 350 |
| | VTEENKELAN ELRRPPLCYH NGVQYRNNEE WTVDSCTECH CQNSVTICKK |
| | 360 370 380 390 400 |
| | VSCPIMPCSN ATVPDGECCP RCWPSDSADD GWSPWSEWTS CSTSCGNGIQ |
| | 410 420 430 440 450 |
| | QRGRSCDSLN NRCEGSSVQT RTCHIQECDK RFKQDGGWSH WSPWSSCSVT |
| | 460 470 480 490 500 |
| | CGDGVITRIR LCNSPSPQMN GKPCEGEARE TKACKKDACP INGGWGPWSP |
| | 510 520 530 540 550 |
| | WDICSVTCGG GVQKRSRLCN NPTPQFGGKD CVGDVTENQI CNKQDCPIDG |
| | 560 570 580 590 600 |
| | CLSNPCFAGV KCTSYPDGSW KCGACPPGYS GNGIQCTDVD ECKEVPDACE |
| | 610 620 630 640 650 |
| | NHNGEHRCEN TDPGYNCLPC PPRFTGSQPF GQGVEHATAN KQVCKPRNPC |
| | 660 670 680 690 700 |
| | TDGTHDCNKN AKCNYLGHYS DPMYRCECKP GYAGNGIICG EDTDLDGWPN |
| | 710 720 730 740 750 |
| | ENLVCVANAT YHCKKDNCPN LPNSGQEDYD KDGIGDACDD DDDNDKIPDD |
| | 760 770 780 790 800 |
| | RDNCPFHYNP AQYDYDRDDV GDRCDNCPYN HNPDQADIDN NGEGDACAAD |
| | 810 820 830 840 850 |
| | IDGDGILNER DNCQYVYNVD QRDTDMDGVG DQCDNCPLEH NPDQLDSDSD |
| | 860 870 880 890 900 |
| | RIGDTCDNNQ DIDEDGHQNN LDNCPYVPNA NQADHDKDGK GDACDHDDDN |
| | 910 920 930 940 950 |
| | DGIPDDKDNC RLVPNPDQKD SDGDGRGDAC KDDEDHDSVP DIDDICPENV |
| | 960 970 980 990 1000 |
| | DISETDERRF QMIPLDPKGT SQNDPNWVVR HQGKELVQTV NCDPGLAVGY |
| | 1010 1020 1030 1040 1050 |
| | DEFNAVDESG TFFINTERDD DYAGFVEGYQ SSSRFYVVMW KQVTQSYWDT |
| | 1060 1070 1080 1090 1100 |
| | NPTRAQGYSG LSVKVVNSTT GPGEHLRNAL WHTGNTPGQV RILWHDPRHI |
| | 1110 1120 1130 1140 1150 |
| | GWKDFTAYRW RLSHRPKTGF IRVVMYEGKK IMADSGPIYD KTYAGGRLGL |
| | 1160 1170 |
| | FVESQEMVFF SDLKYECRDP |
| TMEM53 | Transmembrane protein 53 (SEQ ID NO: 68) |
| Uniprot | 10 20 30 40 50 |
| Q6P2H8 | MASAELDYTI EIPDQPCWSQ KNSPSPGGKE AETRQPVVIL LGWGGCKDKN |
| | 60 70 80 90 100 |
| | LAKYSAIYHK RGCIVIRYTA PWHMVFFSES LGIPSLRVLA QKLLELLEDY |
| | 110 120 130 140 150 |
| | EIEKEPLLFH VESNGGVMLY RYVLELLQTR RFCRLRVVGT IFDSAPGDSN |
| | 160 170 180 190 200 |
| | LVGALRALAA ILERRAAMLR LLLLVAFALV VVLFHVLLAP ITALFHTHEY |
| | 210 220 230 240 250 |
| | DRLQDAGSRW PELYLYSRAD EVVLARDIER MVEARLARRV LARSVDFVSS |
| | 260 270 |
| | AHVSHLRDYP TYYTSLCVDF MRNCVRC |
| VIPR1 | Vasoactive intestinal polypeptide receptor 1 (SEQ ID NO: 69) |
| Uniprot | 10 20 30 40 50 |
| P32241 | MRPPSPLPAR WLCVLAGALA WALGPAGGQA ARLQEECDYV QMIEVQHKQC |
| | 60 70 80 90 100 |
| | LEEAQLENET IGCSKMWDNL TQWPATPRGQ VVVLACPLIF KLESSIQGRN |
| | 110 120 130 140 150 |
| | VSRSCTDEGW THLEPGPYPI ACGLDDKAAS LDEQQTMFYG SVKTGYTIGY |
| | 160 170 180 190 200 |
| | GLSLATLLVA TAILSLFRKL HCTRNYIHMH LFISFILRAA AVFIKDLALF |
| | 210 220 230 240 250 |
| | DSGESDQCSE GSVGCKAAMV FFQYCVMANF FWLLVEGLYL YTLLAVSEFS |
| | 260 270 280 290 300 |
| | ERKYFWGYIL IGWGVPSTFT MVWTIARIHF EDYGCWDTIN SSLWWIIKGP |
| | 310 320 330 340 350 |
| | ILTSILVNFI LFICIIRILL QKLRPPDIRK SDSSPYSRLA RSTLLLIPLE |
| | 360 370 380 390 400 |
| | GVHYIMFAFF PDNFKPEVKM VFELVVGSFQ GEVVAILYCF LNGEVQAELR |
| | 410 420 430 440 450 |
| | RKWRRWHLQG VLGWNPKYRH PSGGSNGATC STQVSMLTRV SPGARRSSSF |
| | QAEVSLV |
| WNT10A | Protein Wnt-10a (SEQ ID NO: 70) |
| Uniprot | 10 20 30 40 50 |
| Q9GZT5 | MGSAHPRPWL RLRPQPQPRP ALWVLLFFLL LLAAAMPRSA PNDILDLRLP |
| | 60 70 80 90 100 |
| | PEPVINANTV CLTLPGLSRR QMEVCVRHPD VAASAIQGIQ IATHECQHQF |
| | 110 120 130 140 150 |
| | RDQRWNCSSL ETRNKIPYES PIFSRGERES AFAYAIAAAG VVHAVSNACA |
| | 160 170 180 190 200 |
| | LGKLKACGCD ASRRGDEEAF RRKLHRLQLD ALQRGKGLSH GVPEHPALPT |
| | 210 220 230 240 250 |
| | ASPGLQDSWE WGGCSPDMGF GERFSKDELD SREPHRDIHA RMRLHNNRVG |
| | 260 270 280 290 300 |
| | RQAVMENMRR KCKCHGTSGS CQLKTCWQVT PEFRTVGALL RSREHRATLI |
| | 310 320 330 340 350 |
| | RPHNRNGGQL EPGPAGAPSP APGAPGPRRR ASPADLVYFE KSPDFCEREP |
| | 360 370 380 390 400 |
| | RLDSAGTVGR LCNKSSAGSD GCGSMCCGRG HNILRQTRSE RCHCRFHWCC |
| | 410 |
| | FVVCEECRIT EWVSVCK |
| XPC | DNA repair protein complementing XP-C cells (SEQ ID NO: 71) |
| Uniprot | 10 20 30 40 50 |
| Q01831 | MARKRAAGGE PRGRELRSQK SKAKSKARRE EEEEDAFEDE KPPKKSLLSK |
| | 60 70 80 90 100 |
| | VSQGKRKRGC SHPGGSADGP AKKKVAKVTV KSENLKVIKD EALSDGDDLR |
| | 110 120 130 140 150 |
| | DFPSDLKKAH HLKRGATMNE DSNEEEEESE NDWEEVEELS EPVLGDVRES |
| | 160 170 180 190 200 |
| | TAFSRSLLPV KPVEIEIETP EQAKTRERSE KIKLEFETYL RRAMKRENKG |
| | 210 220 230 240 250 |
| | VHEDTHKVHL LCLLANGFYR NNICSQPDLH AIGLSIIPAR FTRVLPRDVD |
| | 260 270 280 290 300 |
| | TYYLSNLVKW FIGTFTVNAE LSASEQDNLQ TTLERRFAIY SARDDEELVH |
| | 310 320 330 340 350 |
| | IFLLILRALQ LLTRIVLSIQ PIPLKSATAK GKKPSKERLT ADPGGSSETS |
| | 360 370 380 390 400 |
| | SQVLENHTKP KTSKGTKQEE TEAKGTCRPS AKGKRNKGGR KKRSKPSSSE |
| | 410 420 430 440 450 |
| | EDEGPGDKQE KATQRRPHGR ERRVASRVSY KEESGSDEAG SGSDFELSSG |
| | 460 470 480 490 500 |
| | EASDPSDEDS EPGPPKQRKA PAPQRTKAGS KSASRTHRGS HRKDPSLPAA |
| | 510 520 530 540 550 |
| | SSSSSSSKRG KKMCSDGEKA EKRSIAGIDQ WLEVFCEQEE KWVCVDCVHG |
| | 560 570 580 590 600 |
| | VVGQPLTCYK YATKPMTYVV GIDSDGWVRD VTQRYDPVWM TVTRKCRVDA |
| | 610 620 630 640 650 |
| | EWWAETLRPY QSPEMDREKK EDLEFQAKHM DQPLPTAIGL YKNHPLYALK |
| | 660 670 680 690 700 |
| | RHLLKYEATY PETAAILGYC RGEAVYSRDC VHTLHSRDTW LKKARVVRLG |
| | 710 720 730 740 750 |
| | EVPYKMVKGF SNRARKARLA EPQLREENDL GLEGYWQTEE YQPPVAVDGK |
| | 760 770 780 790 800 |
| | VPRNEFGNVY LFLPSMMPIG CVQLNLPNLH RVARKLDIDC VQAITGFDEH |
| | 810 820 830 840 850 |
| | GGYSHPVTDG YIVCEEFKDV LITAWENEQA VIERKEKEKK EKRALGNWKL |
| | 860 870 880 890 900 |
| | LAKGLLIRER LKRRYGPKSE AAAPHTDAGG GLSSDEEEGT SSQAEAARIL |
| | 910 920 930 940 |
| | AASWPQNRED EEKQKLKGGP KKTKREKKAA ASHLFPFEQL |

Isoforms and variants of the ZNF92, ET-9, or ET-60 genes and gene products can be present in subjects and can be detected, measured, evaluated, and the subjects with such isoforms and variants can be treated by the methods and compositions described herein. Such isoforms and variants can have sequences with between 65-100% sequence identity to a reference sequence, for example with at least at least 65%, at least 70%, at least 80%, at least 90%, at least 95%, at least 96%, at least 97% sequence, at least 98%, at least 99%, or at least 99.5% identity to a sequence described herein or a reference sequence (such as one described in the NCBI or Uniprot databases) over a specified comparison window. Optimal alignment may be ascertained or conducted using the homology alignment algorithm of Needleman and Wunsch, J. Mol. Biol. 48:443-53 (1970).

### Definitions

The “absolute amplitude” of correlation expressions means the distance, either positive or negative, from a zero value; i.e., both correlation coefficients −0.35 and 0.35 have an absolute amplitude of 0.35. ZNF92, ET-9, or ET-60 genes, “Status” means a state of gene expression of a set of genetic markers whose expression is strongly correlated with a particular phenotype. For example, “ZNF92 status” means a state of gene expression of a set of genetic markers (e.g., ET-9 or ET-60 markers) whose expression is strongly correlated with that of the ZNIF92 gene, wherein the expression pattern of these (e.g. ET-9 or ET-60) can differ detectably between tumors expressing the ZNF92 and tumors not expressing ZNF92.

“Good prognosis” means that a patient is expected to have longer overall survival (OS), or progression-free survival (PFS), or disease-specific survival (DSS) or recurrence-free survival (RFS) compared to “poor prognosis” patients. These metrics are typically described by National Cancer Institute (NCJ) as overall survival (OS), or progression-free survival (PFS) which is the length of time during and after the treatment of cancer, that a patient lives with the disease but it does not get worse, or disease-specific survival (DSS) that is the percentage of people in a treatment group who have not died from their cancer in a defined period of time, or recurrence-free survival (RFS) that is length of time after primary treatment for a cancer ends that the patient survives without any signs or symptoms of that cancer, also called as disease-free survival (DIFS), or relapse-free survival (see website at cancer.gov/publications/dictionaries/cancer-terms/def/rfs)

“Poor prognosis” means that a patient is expected to have a shorter overall survival (OS), or progression-free survival (PFS), or disease-specific survival (DSS) or recurrence-free survival (RFS) compared to “good prognosis” patients.

“Marker” means an entire gene, mRNA, EST, or a protein product derived from that gene, where the expression or level of expression changes under different conditions, where the expression of the gene (or combination of genes) correlates with a certain condition, the gene or combination of genes is a marker for that condition.

“Marker-derived polynucleotides” means the RNA transcribed from a marker gene, any cDNA, or cRNA produced therefrom, and any nucleic acid derived therefrom, such as synthetic nucleic acid having a sequence derived from the gene corresponding to the marker gene.

A “similarity value” is a number that represents the degree of similarity between two things being compared. For example, a similarity value may be a number that indicates the overall similarity between a patient's expression profile using specific phenotype-related markers and a control specific to that phenotype (for instance, the similarity to a “good prognosis” template, where the phenotype is a good prognosis). The similarity value may be expressed as a similarity metric, such as a correlation coefficient, or may simply be expressed as the expression level difference, or the aggregate of the expression level differences, between a patient sample and a template.

The present description is further illustrated by the following examples, which should not be construed as limiting in any way.

### Example 1: HDACI and HDAC7 Co-Regulated Genes

HDACI and HDAC7 each regulate over 3,000 to 5,000 genes in different breast cancer cells, making the analysis of their downstream targets challenging.

However, gene set enrichment analysis (GSEA) was used to identify overlap among expression signatures that could be used to reveal underlying biological processes. Nine gene set collections of the Molecular Signatures Database (MSigDB) with 32,274 gene sets were used to explore the cellular pathways, processes, and genes that may be associated with the HDAC1/7-superenhancer (SE) upregulated gene signature. The top ten gene sets having the most significant overlap with HDAC1/7-SE upregulated genes in the MSigDB Hallmark collection (H, n=50) included mRNA signatures associated with epithelial-mesenchymal transition (p=2.28 e⁻⁷), K-Ras signaling (p=:3.24 ^{e-6}), apoptosis (p=1.52^{c-4}), Wnt-B-catenin signaling (p=3.06^{e-4}) hypoxia (p=4.14^{e-4}) and p53 pathway (p=: 4.14^{c-4}). All of these pathways have been implicated in metastasis or poor cancer outcome. Hence, their identification as the top-ranking signatures that overlap with the HDAC1/7-SE upregulated gene set was notable.

In the MSigDB Curated gene set (C2) collection, the top ten most enriched gene sets with significant overlap with HDAC1/7-SE upregulated genes included HDAC1 targets (p=:2.66° i) and HDAC1 and HDAC2 targets (p=:2.37^{e-6}). Identification of HDAC1 targets among the 6,290 gene sets in C2 corroborated the experimental results.

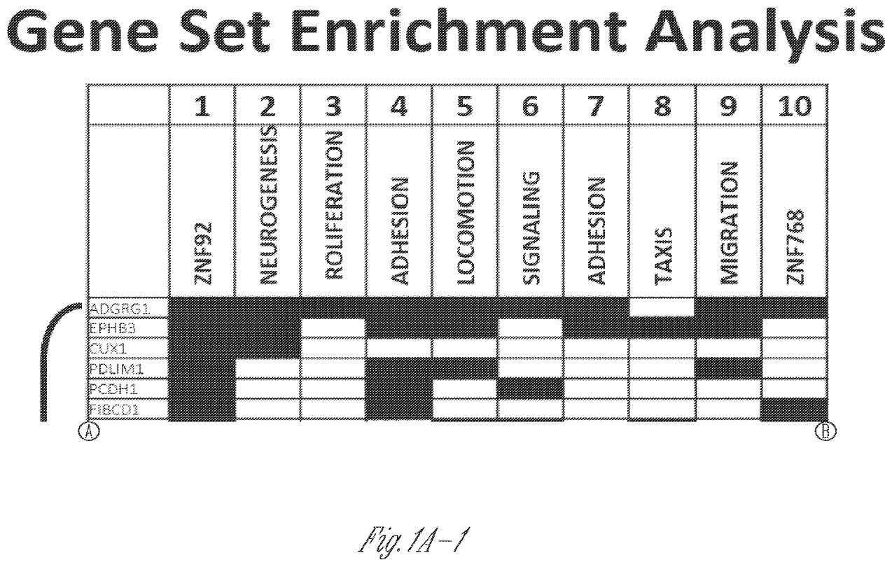

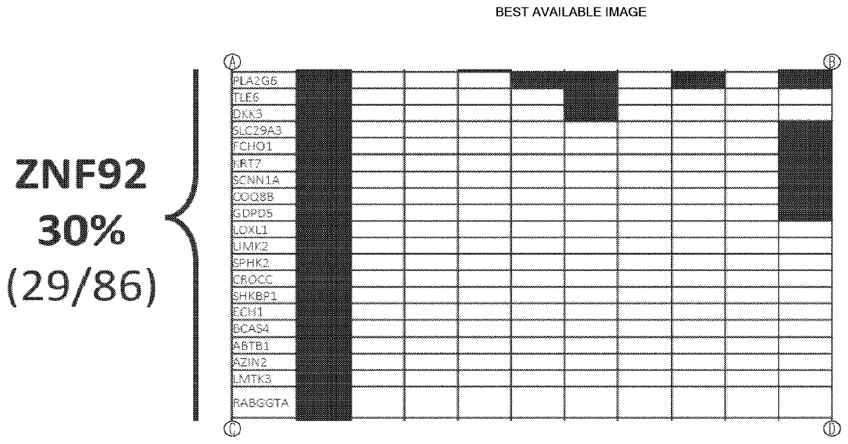

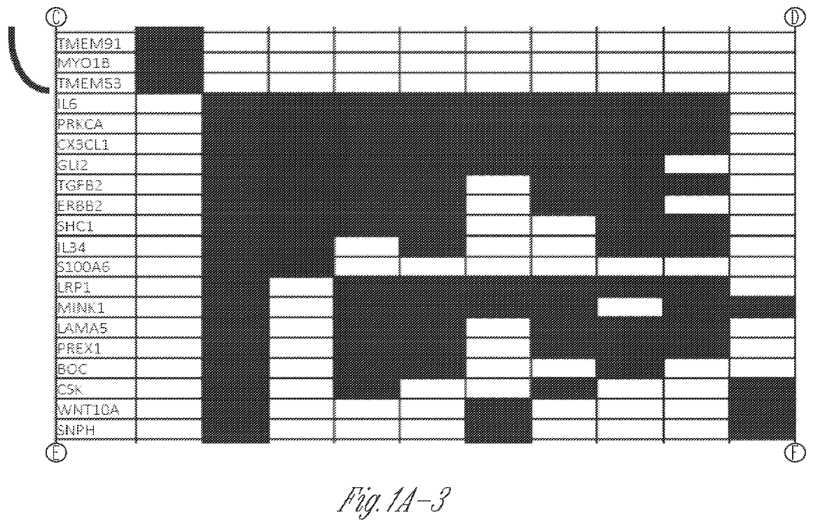

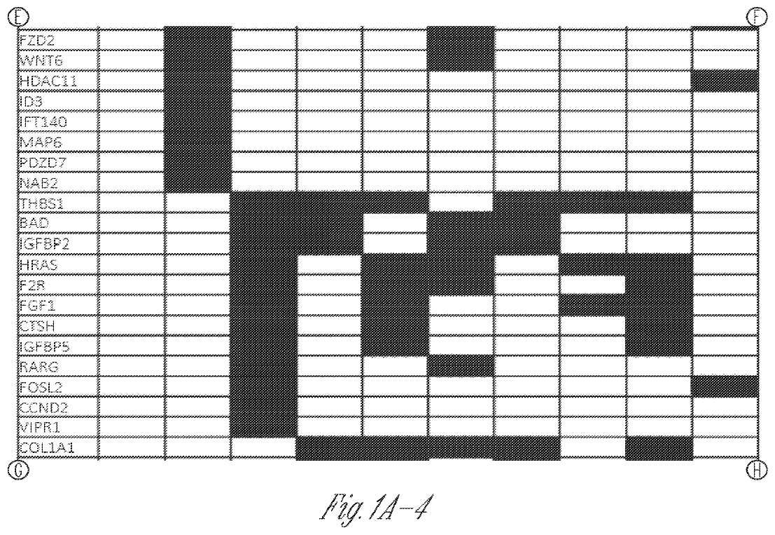

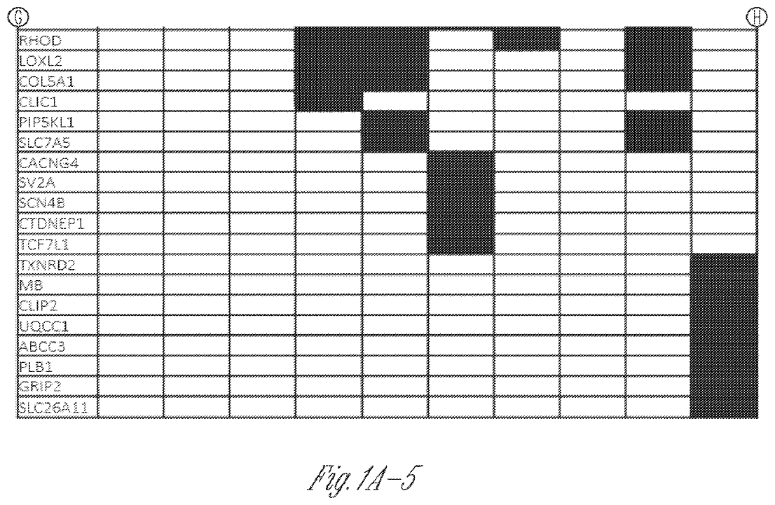

Next, a combined GSEA was carried out of C3-C8 in MSigDB, which includes gene ontology, oncogenic, immunologic, cell type, regulatory and cancer gene sets (n=16,663). This analysis revealed that the top ten enriched gene sets included a majority of HDAC1/7-SE upregulated genes (86/125), and among these, the genes with a ZNF92 binding site ranked #1 out of 16,663 signatures (FIG. 1A).

### Example 2: ZNF92 Expression in Breast Cancer

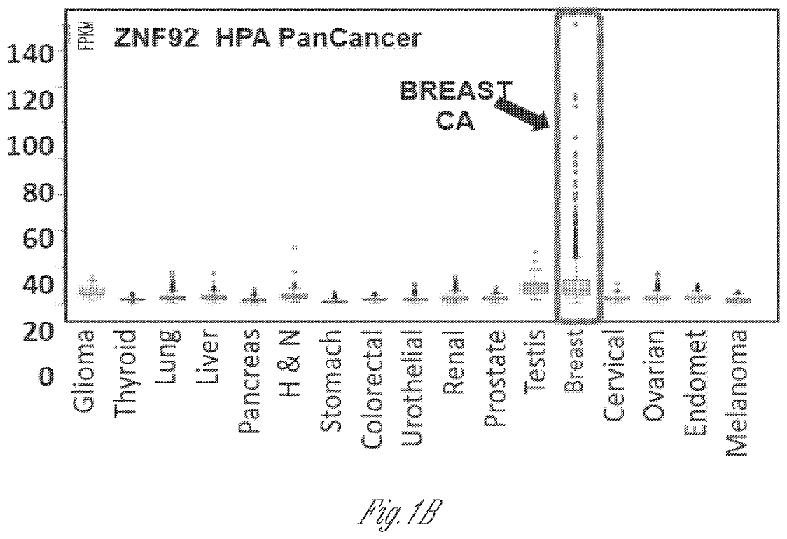

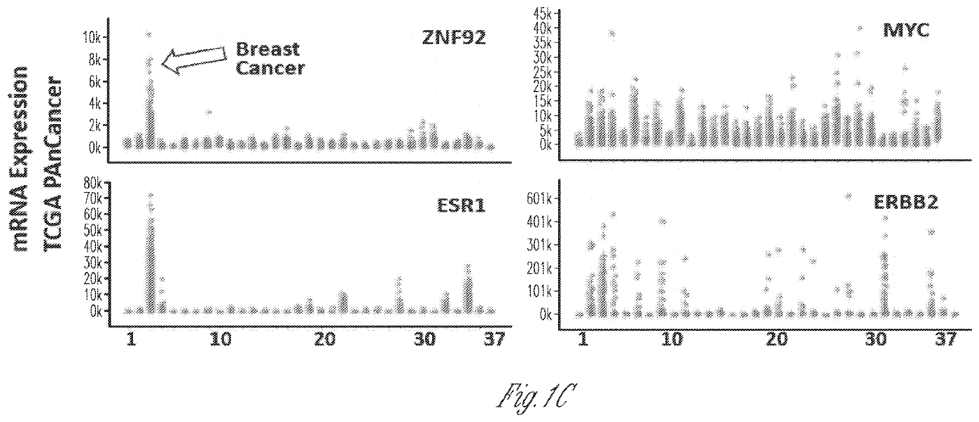

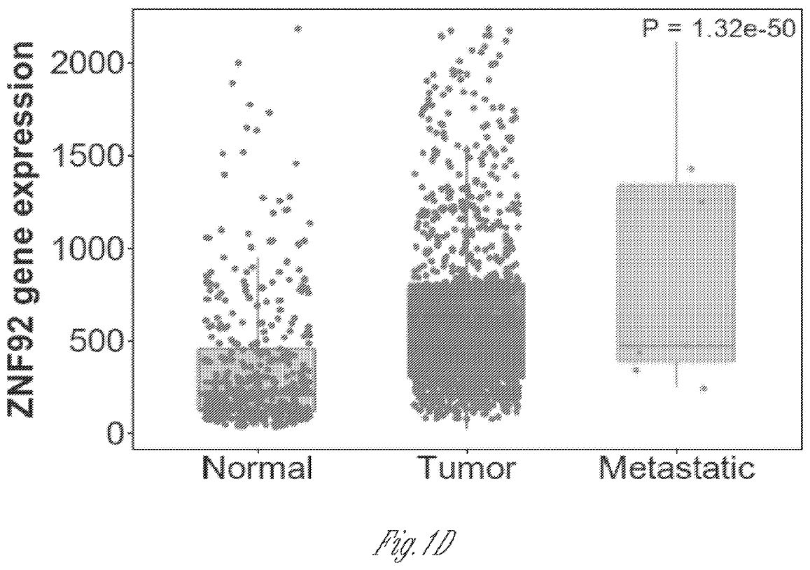

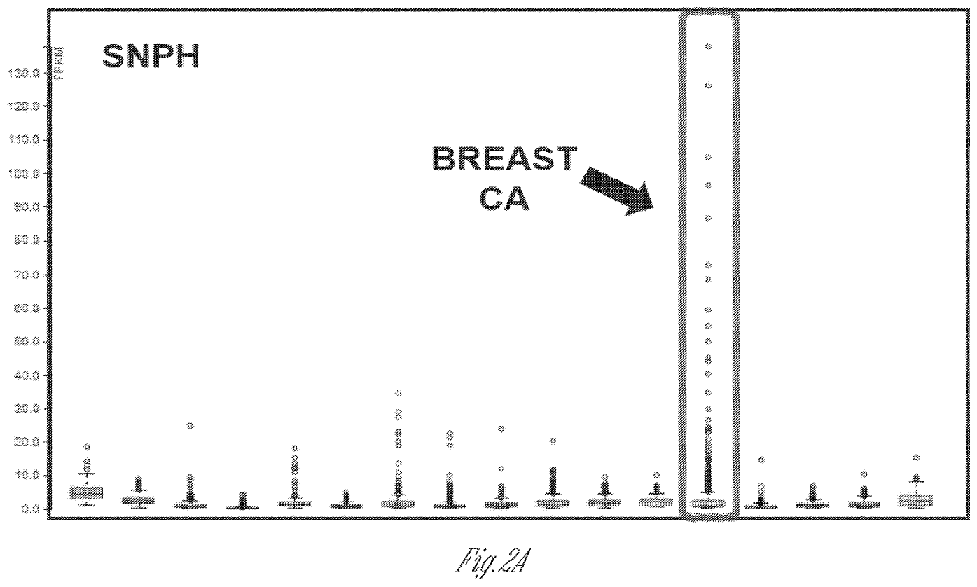

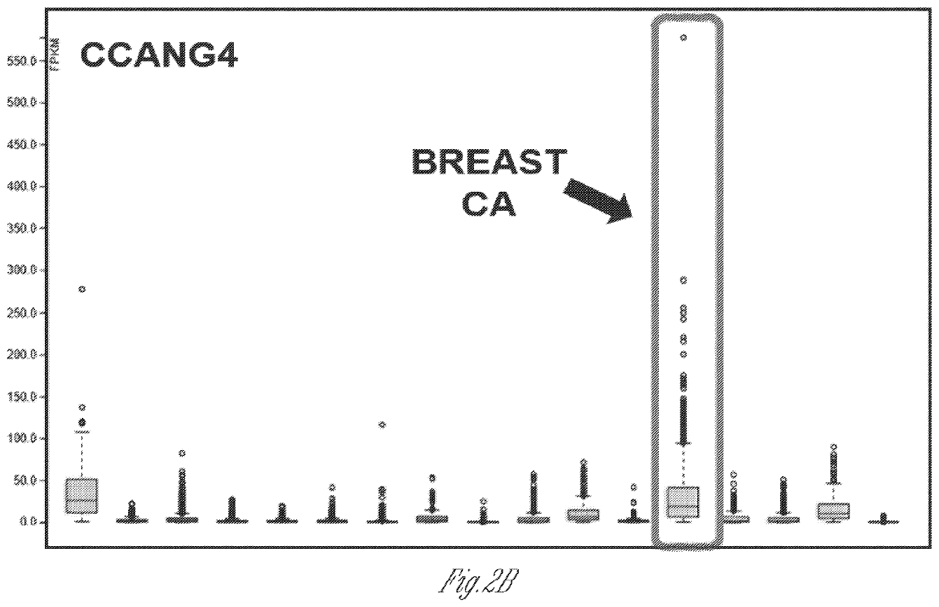

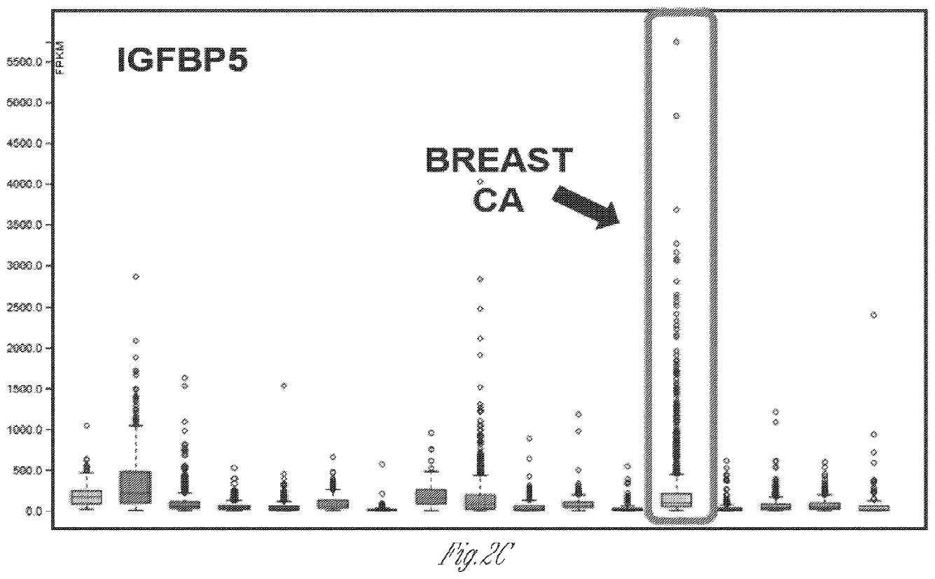

Surprisingly, the inventors determined that ZNF92 is distinctively over-expressed in breast cancer compared to all other cancer types in the Human Protein Atlas (HPA), The analysis of RNAseq data from seventeen cancer types, including 7,932 tumor samples in the-PA, revealed breast cancers with strikingly high ZNF92 expression (FIG. 1B). In contrast, ZNF768 that ranked 10th in the GSEA does not appear to have breast cancer specificity (FIG. 2). The extraordinary breast cancer-specific expression of ZNF92 in EPA was confirmed among the 37 cancer types represented in the TCGA PanCancer dataset that includes 10,528 tumor samples (Ponten et al 270 (5), 428-446, J Intern Med, 2011). Importantly, ZNF92 over-expression appears to be even more specific for breast cancer compared to benchmarks such as estrogen receptor (ER) and HER2 (FIG. 1C). In this analysis most of the oncogenes do not have any tumor type specificity (FIG. 1C). Also, using TNMplot online tools (website at //tnmplot.com/analysis/) the inventors determined that ZNF92 expression is increased between normal breast and breast tumors, with further increase in metastatic samples (FIG. 1D) (Bartha and Gyorffy, Int J Mol Sci 22(5), 2021)

ZNF92 is an exceptionally unexplored protein, as it is only mentioned in a single paper as one of eleven genes with potential changes in their splicing patterns after treatment of a liver cell line HepG2 with cholesterol-lowering drug atorvastatin (Storno et al. PloS One 9 (8) e105836, 2014). There are no studies linking ZNF92 with any cancer. Therefore, discovering the striking breast cancer specific over-expression of ZNF92 was rather unexpected.

Interestingly, several other HDAC1/7-SE upregulated targets, such as SNPH, CCANG4, PREXI, IGFBP5, IL34 and BCAS4 also demonstrate remarkable level of breast cancer associated overexpression, providing additional support for the relevance of the ET-9 and ET-60 signatures (FIG. 2).

### Example 3: ET-60 and ET-9 Signatures

The inventors then determined that a sixty gene subset of the HDAC1&7-SE upregulated genes, including 22 targets of ZNF-92, referred to herein as Epigenetic Tumor (ET-60) signature (Table 2) correlated significantly with breast cancer patient outcome as analyzed by using SurvExpress online tools (see website at (bioinformatica.mty.itesm.mx:8080/Biomatec/SurvivaXvalidatorjsp) (Aguirre-Gamboa et al.; 8 (9), e74250, PLoS One, 2013).

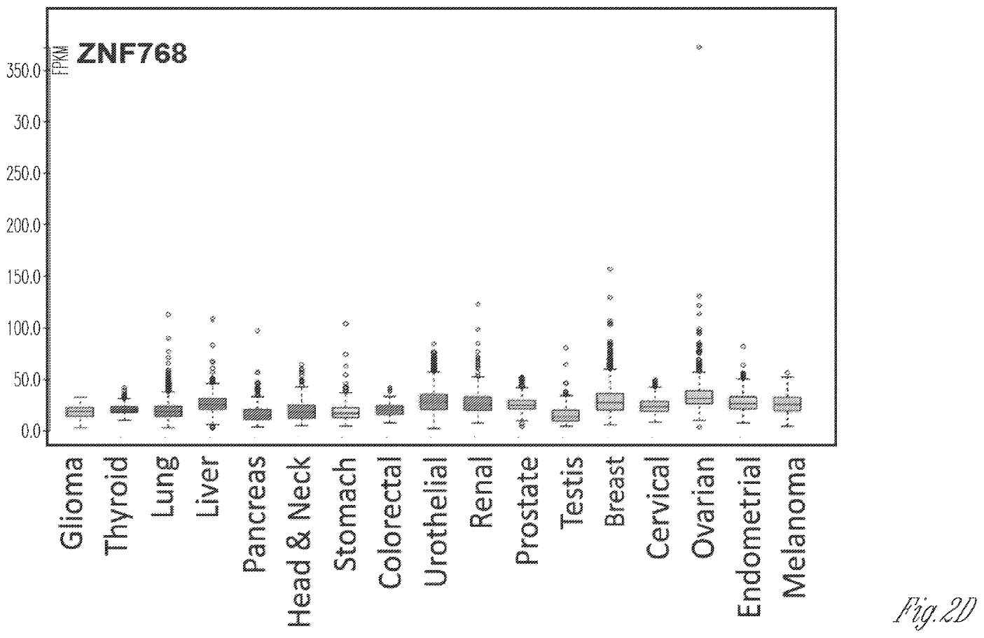

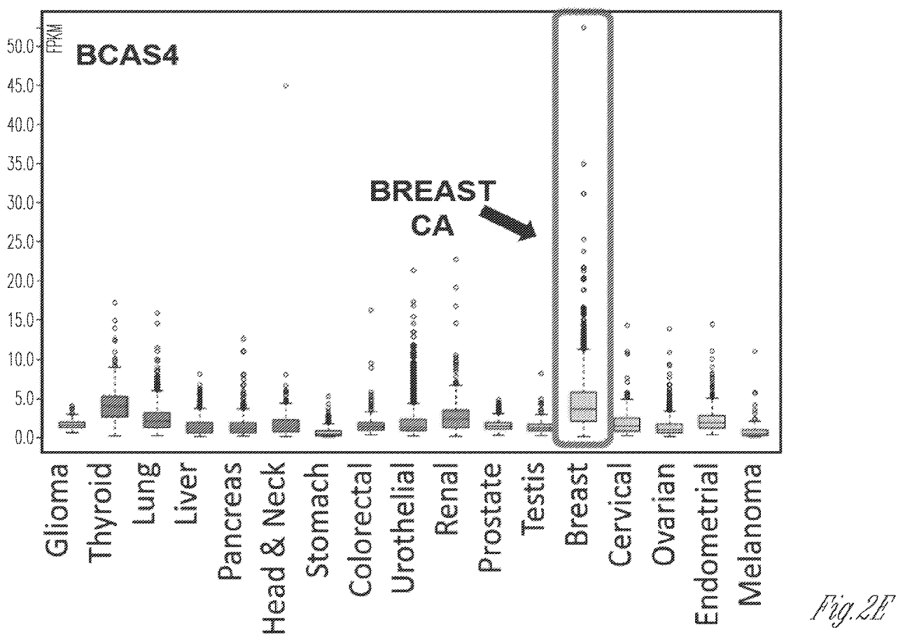

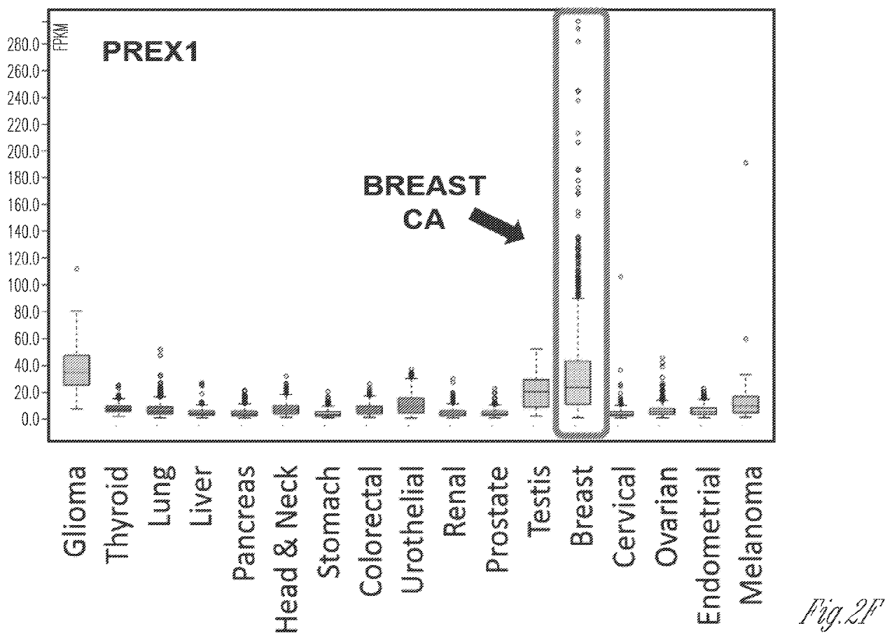

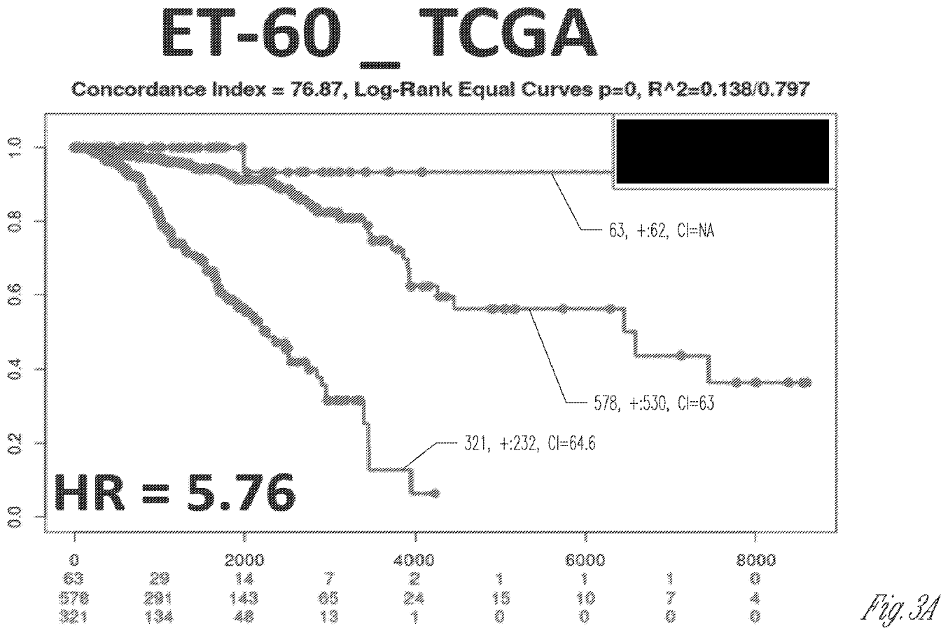

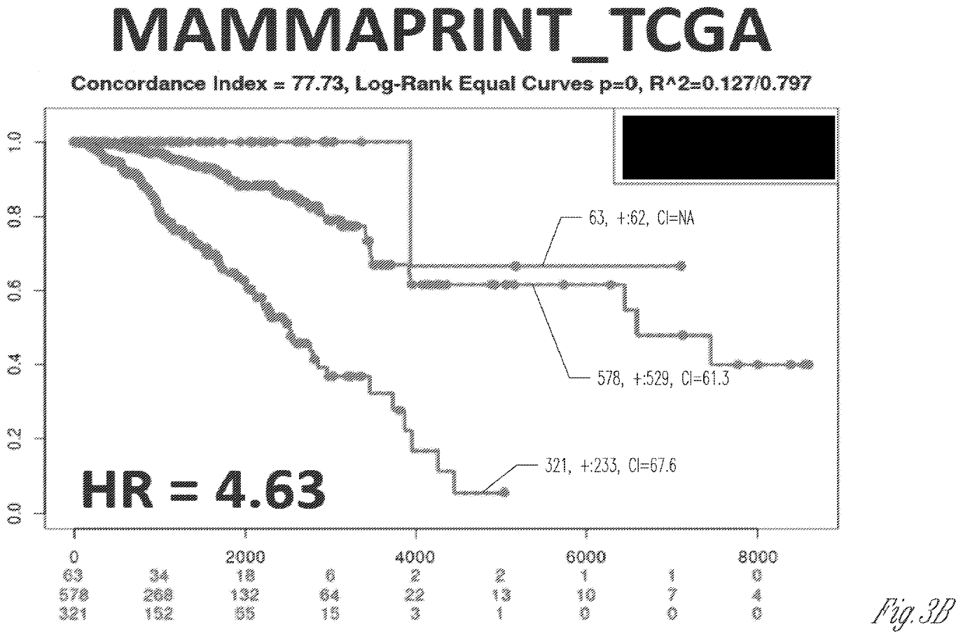

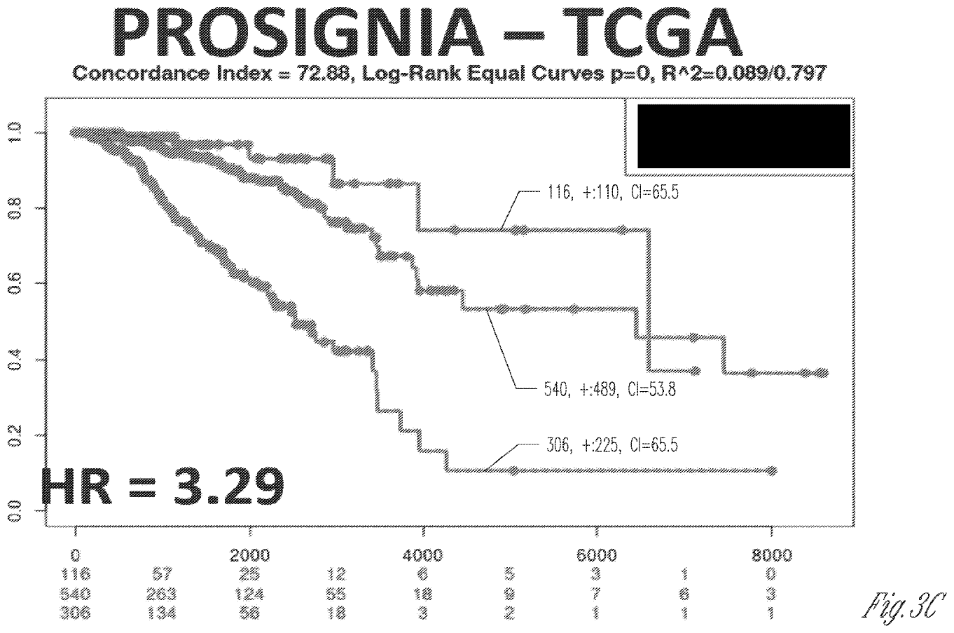

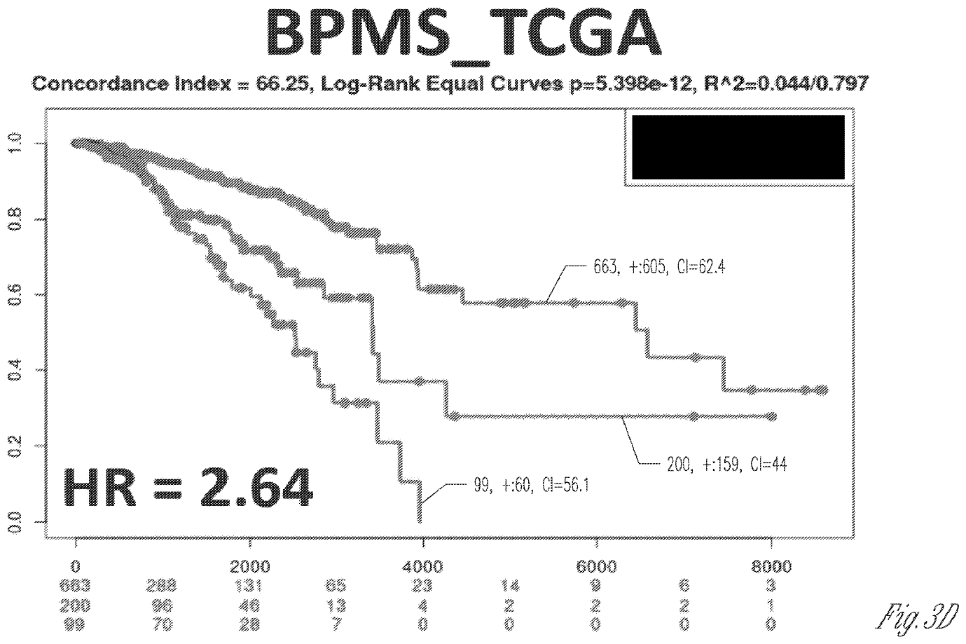

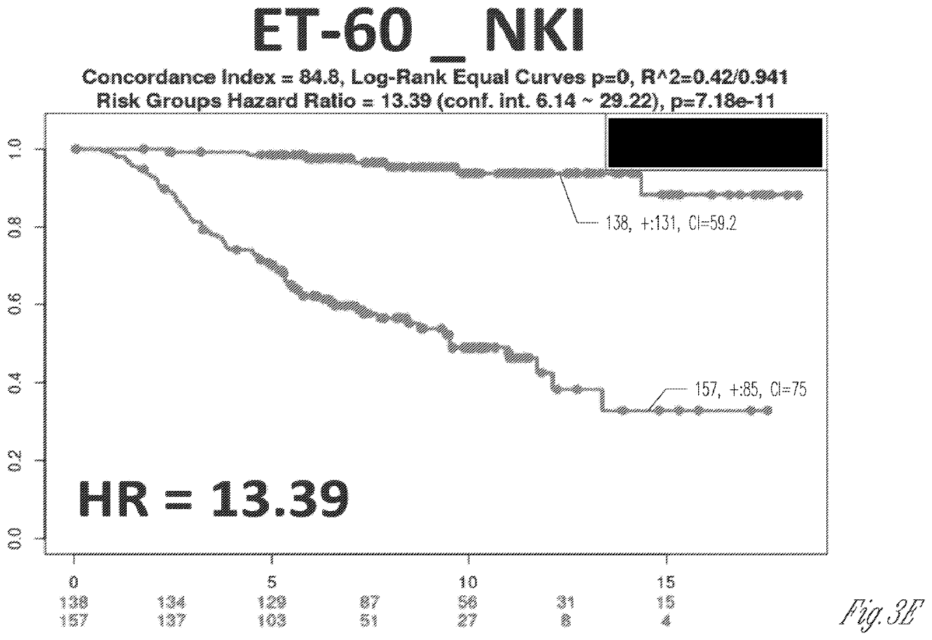

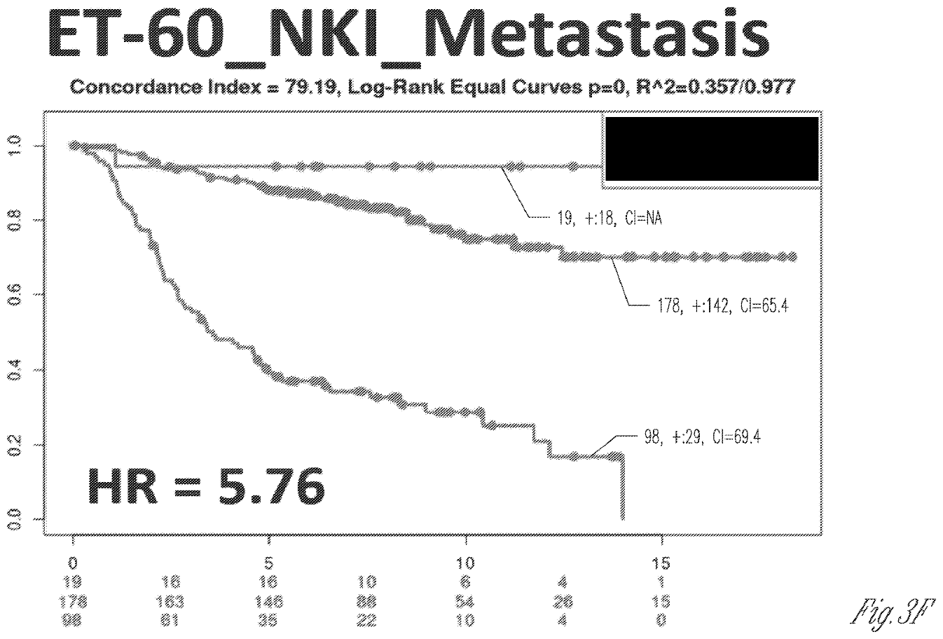

High ET-60 expression was associated with a greater hazard ratios 5.76 (C: 4.0-8.2)(Aguirre-Gamboa et al; 8 (9), e74250, PLoS One, 2013), compared to the commercially available signatures, including a 70-gene signature (Mammaprint, HR=4.6), the 50-gene signature PAM50 (Prosignia, HR=3.2) and a 25 gene signature BPMS (HR=2.6) (FIG. 3) (Lee et al. PLoS One 8(12) e82125; Nunes et al. NCI Cancer Spectr 1(1) pkx008, 2017). The hazard ratio (HR) is defined as a comparison between the probability of events in a treatment group, compared to the probability of events in a control group. For example, a hazard ratio of 3 means that three times the number of events are seen in the treatment group at any point in time.

Moreover, ET.-60 predicted shorter lag-time to metastasis in two additional datasets (NKI, HIR=5.7, and SKI HR-9.5^{e9}).

Signatures approaching 100 genes may have increased random associations (Venet et al. PloS Comput Biol. 7(10) e1002240, 2011). Translating these results into a clinical test would be more practical with a smaller number genes that can be measured with a variety of technologies. Hence, with further analysis the inventors identified the nine-gene subset from the initial sixty-eight genes, henceforth referred as Epigenetic Tumor (ET-9) signature (Table 1).

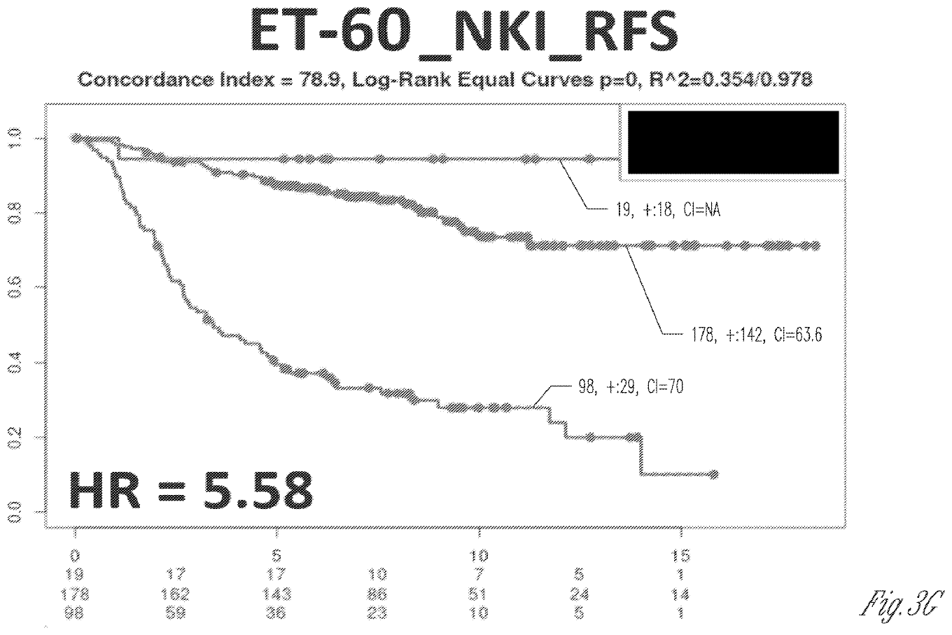

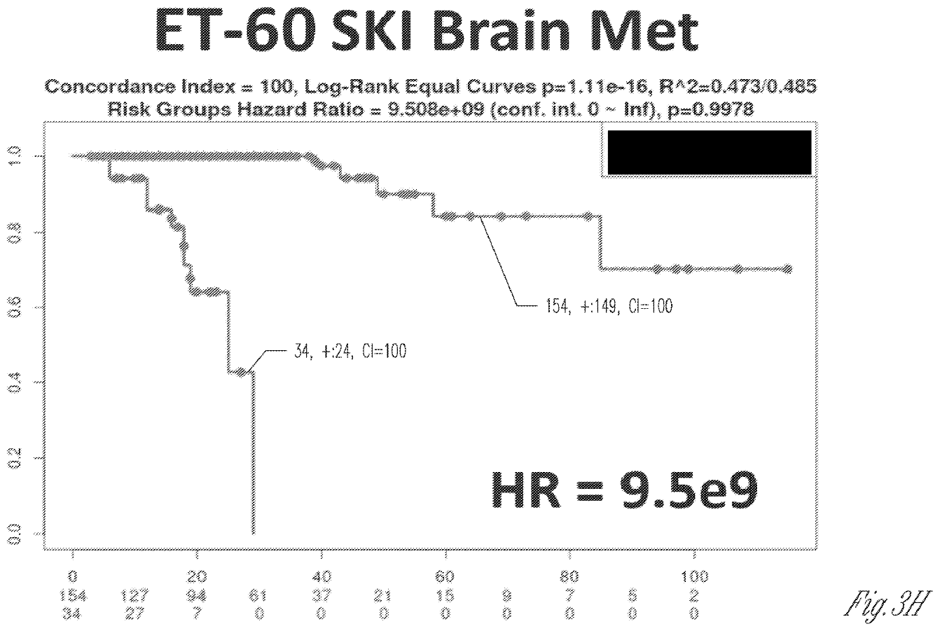

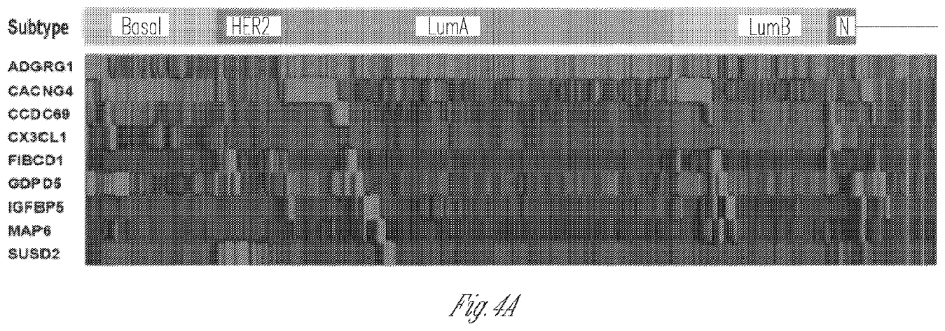

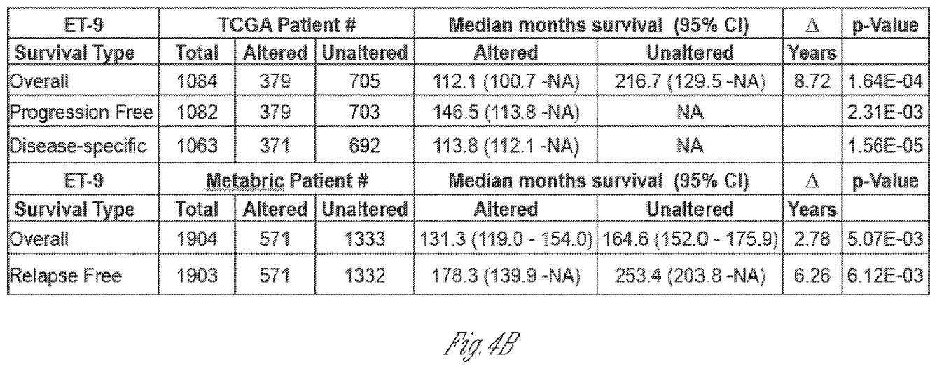

Using cBioPortal online tools (see website at cbioportal.org/) (Gao et al. Sci Signal 6(269 pl1 (2013)) the inventors found that the ET-9 genes were over-expressed in all subtypes of breast cancer in the Breast Invasive Carcinoma (TCGA, PanCancer Atlas) dataset (FIG. 4A)

### Example 4: Altered ET-9 Signature is Prognostic of Shorter Survival

This Example illustrates that the ET-9 signature can be used to identify which subjects (e.g., breast cancer patients) have a poor prognosis, thereby indicating that those subjects should have further treatment.

### Methods

Two different software packages were used to analyze the survival data, SurvExpress and Kaplan-Meier Plotter.

The prognostic significance of the ET-9 genes was individually analyzed using metasurvival analysis (see website at gent2.appex.kr/gent2/; Park et al. BMC Med Genomics 12 (Suppl 5) 101, 2019).

The SurvExpress analysis was carried out selecting; (a) censored survival days, (b) without stratification, (c) heat map by prognostic index, (d) Network none, (e) no imputation, (f) no quantization (g) advanced check, (h) attribute plot check with default options for other variables. Depending on the analysis two or three risk groups were selected, which were determined by prognostic index (risk score) estimated by beta coefficients multiplied by gene expression values. The risk groups are split by the median of the prognostic index generating risk groups of the similar number of samples.

Alternatively, Maximize Risk Groups option was used where, risk group splitting was optimized using an algorithm that decides where the partitions should be made to maximize the statistical significance of the separation of risk groups as described in the tutorial “First, the algorithm start by partitioning samples by same-size risk groups. Then a p-value is estimated by changing the cut-off point one group at the time until a certain limit (five samples or L % of samples where L=20/#risk groups). The new cut-off point is chosen so that the p-value is minimum. This process is repeated until no changes are needed” (Aguirre-Gamboa R et. sL., PLoS One. 2013 Sep. 16; 8(9):e74250. doi:10.1371/joturnal pone.0074250. PMID: 24066126; PMCIID: PMC3774754.

The Kaplan-Meier Plotter (kmplot corn/analysis/index.php?p=:service&cancer=breast) was performed using the following parameters:
Survival: RFS
Auto select best cutoff: checked
Follow up threshold: all
Censor at threshold: checked
Compute median over entire database: false
Probe set option: user selected probe set and mean expression of
selected genes
Invert HR values below 1: not checked

Several alternative approaches were tested to define comparison cohorts (a) quantile cut-off at the median, upper, and lower quartiles, (b) trichotomizing (Ti vs. T3 or Q vs Q4) which involves assigning the data into three cohorts and then omit the middle cohort, or (c) using the best available cut-off value. The results shown are with the best available cut-off value. However, it is possible to generate similar results using the quantile and trichotomizing approaches in some cases depending on the dataset. As described in the tutorial, “To find the best cutoff, [we]iterate over the input variable values from the lower quartile to the upper quartile and compute the Cox regression for each setting. The most significant cut-off value is used as the best cutoff to separate the input data into two groups.” The tutorial further stated, “In case the generated cut-off values are ambiguous (e.g., multiple cut-off values deliver very low P values), the cut-off value corresponding to the highest FR is used” (Ldnczky, Andras, and BalAzs Gvrffy. “Web-Based Survival Analysis Tool Tailored for Medical Research (KMplot): Development and Implementation.” Journal of med/cal Internet research vol. 23,7 e27633. 26 Jul. 2021, doi:10.2196/27633).

### Results

As illustrated in FIG. 4B-4D, the ET-9 signature was associated with shorter overall survival (p=1,63^{c-4}), progression free survival (p=2.31^{c-3}), and disease-specific survival (p=1.56-).

These results were confirmed in the METABRIC breast cancer dataset where ET-9 signature is associated with shorter overall (p=₅0.O₇-3) and relapse free survival (p=6.12^{e3}) (FIG. 4B). The BIC_TCGA and METABRIC datasets include 2,988 patients with over 20 years of follow up (cBioPortal) (Gao et al. Sci Signal 6 (269) pil, 2013) Analysis of these data revealed that the patients with an altered ET-9 signature have 8.7 years shorter median overall survival in the TCGA cohort (9.3 years vs. 18 years) and 6.2 year shorter relapse-free survival in the METABRIC cohort (14.9 years vs. 21.1 years) (FIG. 4 B).

It is worth noting that 6 to 9 year differential in median survival is not typical for breast prognostic signatures and demonstrates the significance of ET-9 signature.

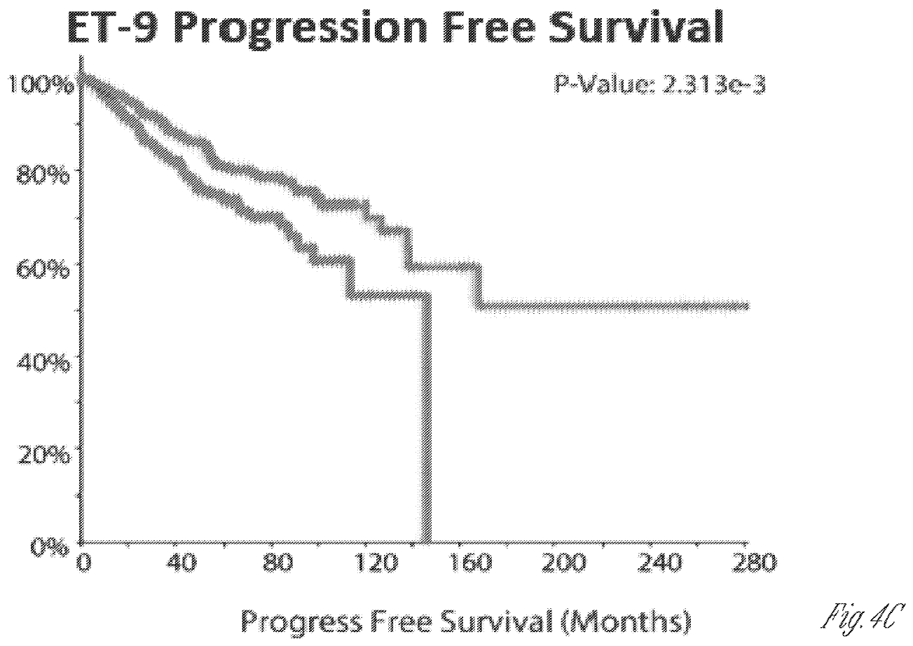

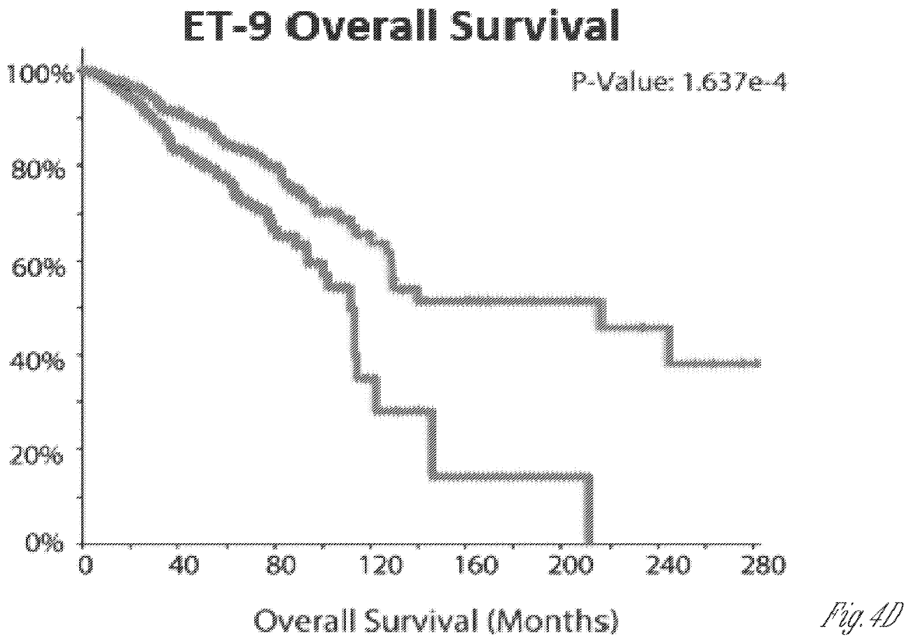

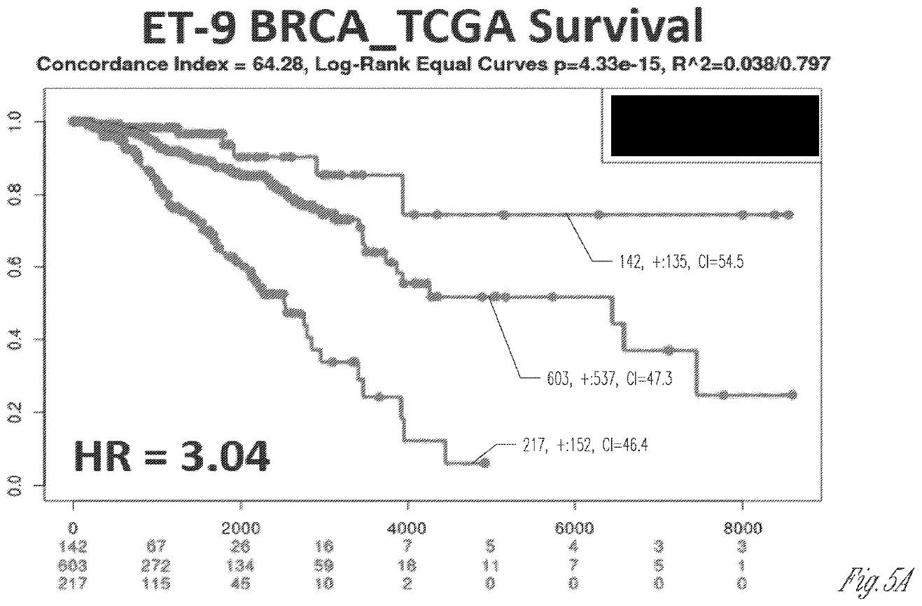

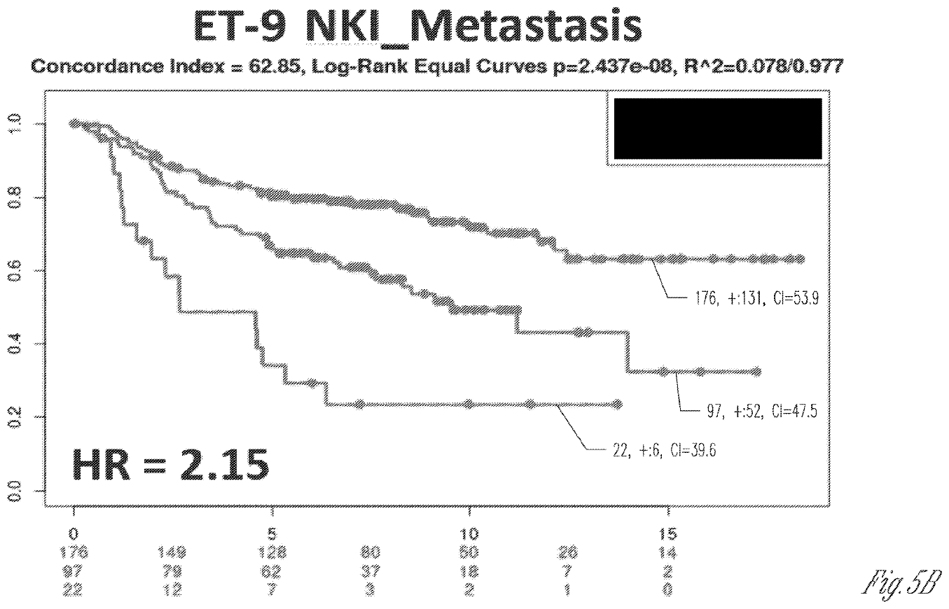

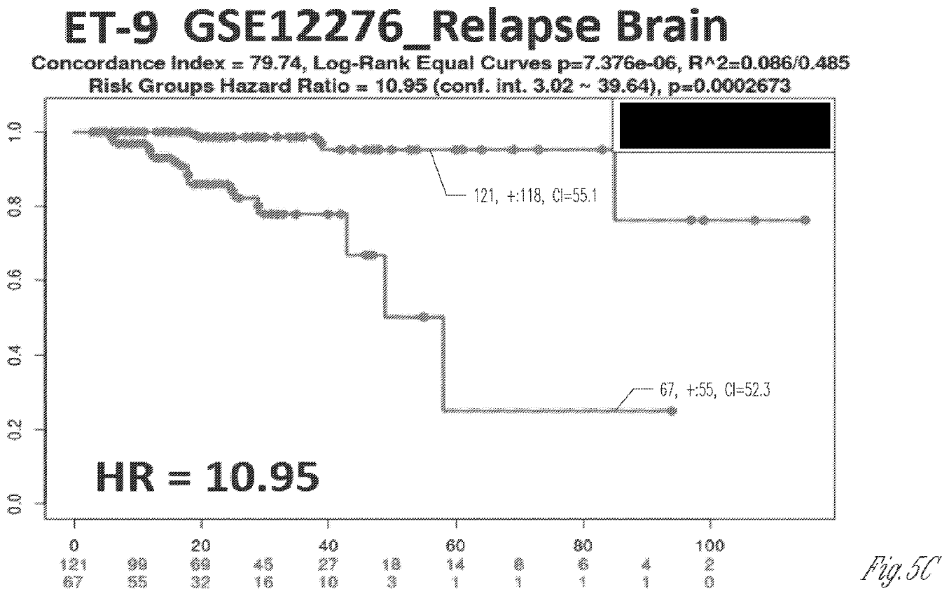

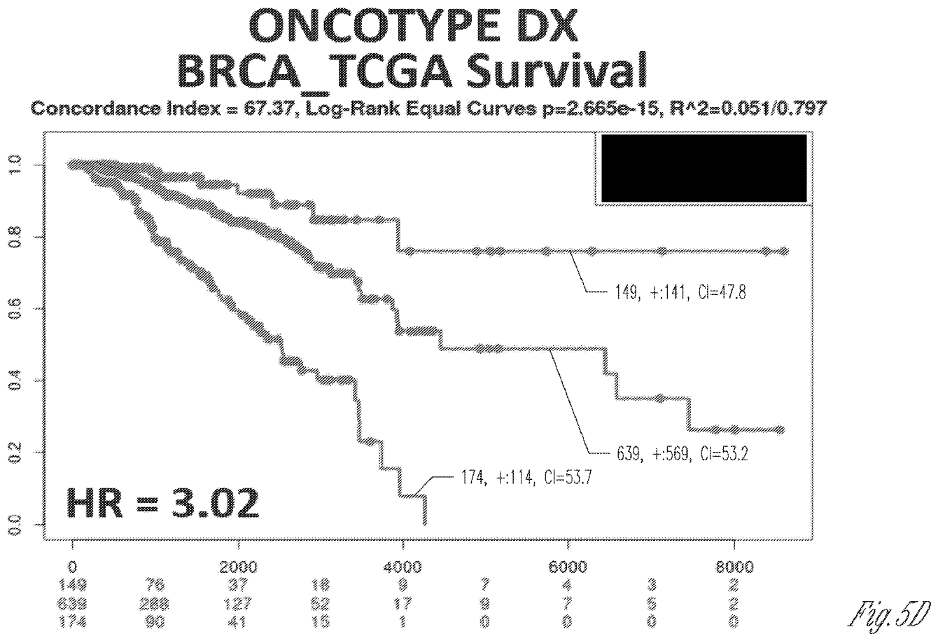

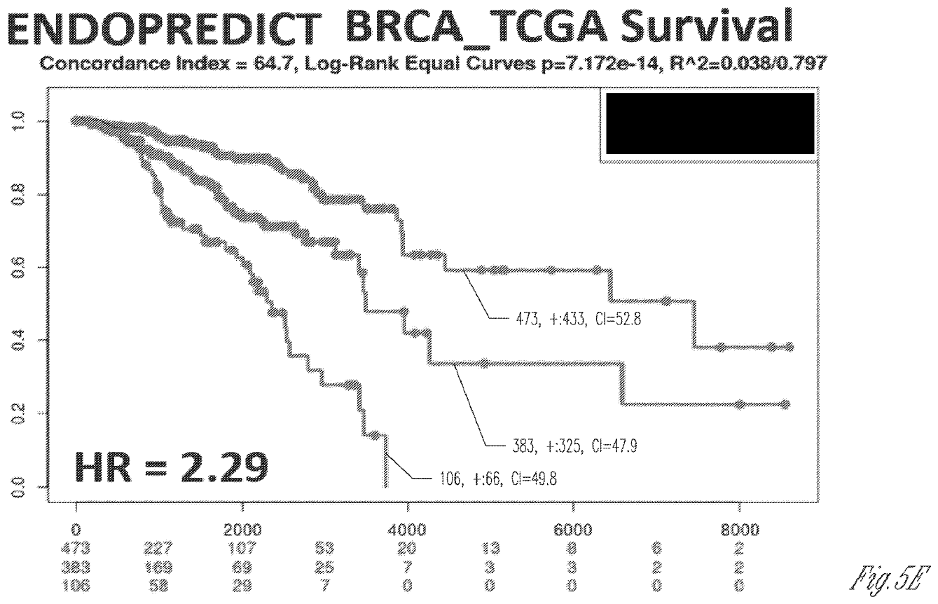

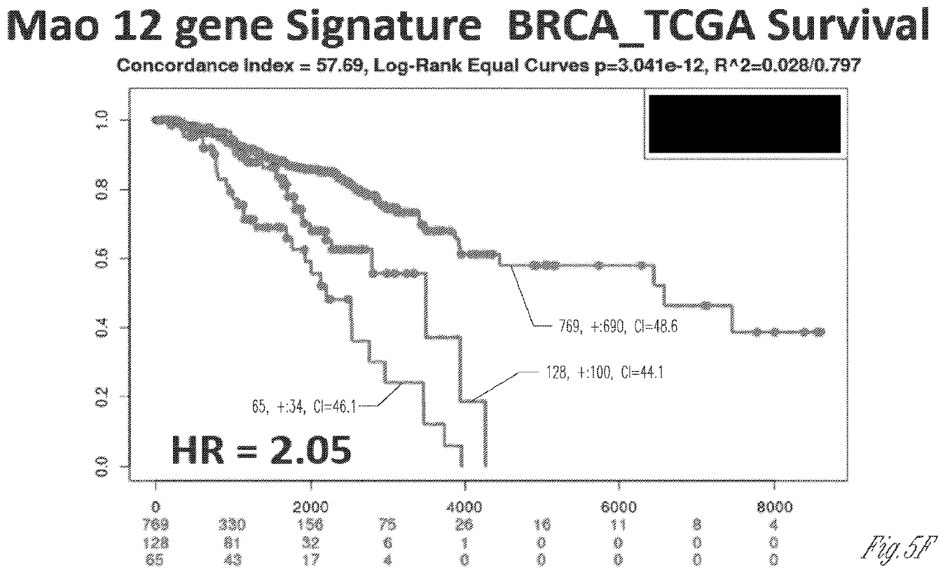

The prognostic significance of the ET-9 signature was also confirmed in three additional datasets and analytical tools (SurvExpress) (Aguirre-Gamboa et al.; 8 (9), e74250, PLoS One, 2013) (see website at gent2.appex.kr/gent2/; Park et al. BMC Med Genomics 12 (Suppl 5) 101 (2019)). This analysis shows that ET-9 correlates with overall survival in TCGA dataset (HR=3.04), outperforming commercial tests including Oncotype DX (I-R=2.2), and Endopredict (HR=2.2) (FIG. 5). Moreover, ET-9 correlates with metastasis in NKI dataset (HJR:=2.15), as well as brain relapse in the GSE12276 dataset (HR=10.95) (FIG. 5).

Note that there was no significant survival association with any single gene by itself in the ET-9 signature. Therefore, the synergistic combined prognostic power of the ET-9 signature was unexpected and is not simply an additive increase in the prognostic value of the individual ET-9 genes.

### Example 5: Proliferation Signature

Even in the era of molecular diagnostics, the histological grading of breast cancer remains to be one of the most powerful prognostic tools. For example, the relative hazard ratio between grade I vs. grade III cancers (HR=3.32-5.1), is greater than the impact of ER expression (HR=2.5-3.71), HER2 amplification (HR=l.27-2.2), or TNBC/basal subtype (HR=1.87-2.2) (Giuliano et al. C A Cancer J Clin 67: 290-303 (2017): Saadatmand et al., BNMJ 351h4901 (2015).

The breast cancer grading system combines three attributes of tumors: (i) the mitotic count as a measure of proliferation, (ii) the extent of tubule formation as a measure of architectural tissue differentiation, and (iii) the degree of nuclear pleomorphism as a measure of cellular differentiation.

Most molecular signatures appear to be surrogate measure of proliferation (Sotiriou and Pusztai; 360 (8), 790-800, N Engl J Med, 2009). For example, Sole et al, reported that proliferation associated genes are over-represented in 22. out of 2.4 breast prognostic signatures (Sole et al.; 4 (2), e4544, PLoS One, 2009). The inventors found that a great majority of the top 20 gene sets associated with commercially available Prosignia and Mammaprint tests are associated with cell proliferation, 90% (9/10) and 70% (7/10) respectively. Venet et al., reported that after removing proliferation associated genes (n=131) in 47 published signatures, their association with outcome dropped dramatically (Venet et al.; 7 (10), e1002240, PLoS Comput Biol, 2011). For example, adjusting for proliferation reduced the 70-gene Mammaprint signature HR from 5.4 down to 1.9 (Venet et al.; 7 (10), e1002240, PLoS Comput Biol, 2011). However, because there is no overlap between ET-9 and ET-60 with the 131 gene proliferation signature of Venet et al., there was no reduction in HR with this adjustment.

The results described herein bring into question the biological interpretation of the proliferation associated breast cancer signatures, but they do not necessarily diminish their usefulness in the clinic. Nonetheless, the results described herein also show that there is significant room for improvement in the area of determining breast cancer diagnosis and prognosis. The prognostic signatures of ET-9 and ET-60, which are independent of proliferation, are particularly useful for such diagnosis and prognosis.

### Example 6: Breast cancer subtype and stage

Although, the grade and lymph node stage are still powerful prognostic features of breast cancer (Johansson et al.; 23 (1), 17, Breast Cancer Res, 2021), existing commercial prognostic signatures (Oncotype DX, Prosignia, Endopredict) are useful only in early stage, small ER-positive/HER-negative and lymph node-negative breast cancers (Nunes et al JNCI Cancer Spectr 1(1) pkx008, 2017).

ER-positive breast cancers include high-grade tumors with increased proliferative index that have a worse outcome compared to low grade ER-positive tumors with a low proliferation rate. As most of the prognostic signatures have been associated with proliferation, their ability to identify ER-positive tumors with high proliferation index is not surprising. However, the prognostic power of proliferation may be more limited in other subtypes of breast cancer.

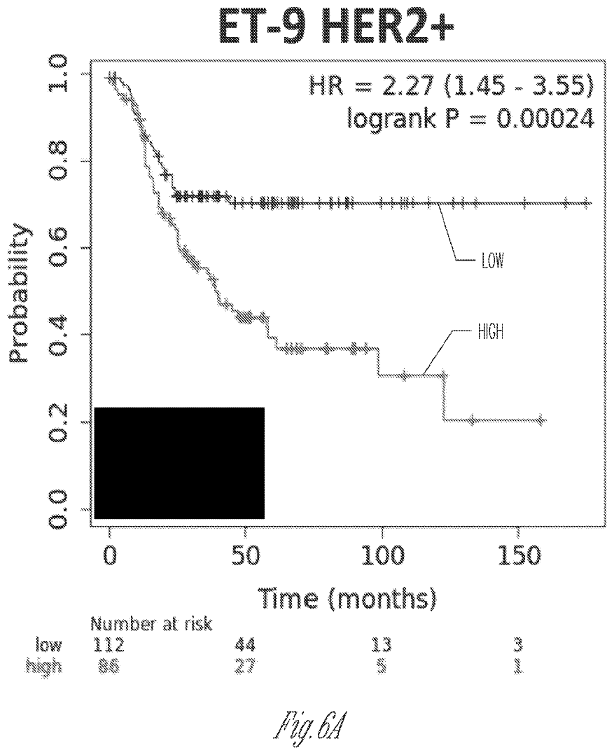

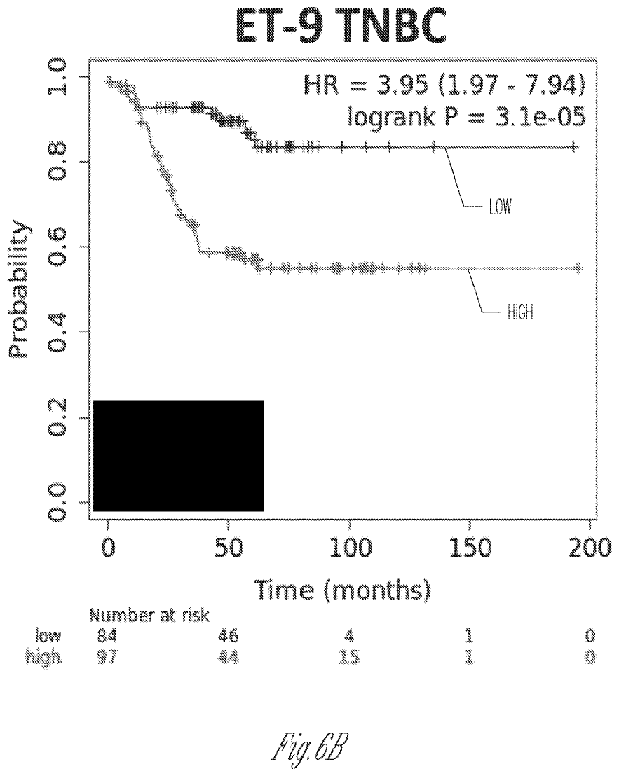

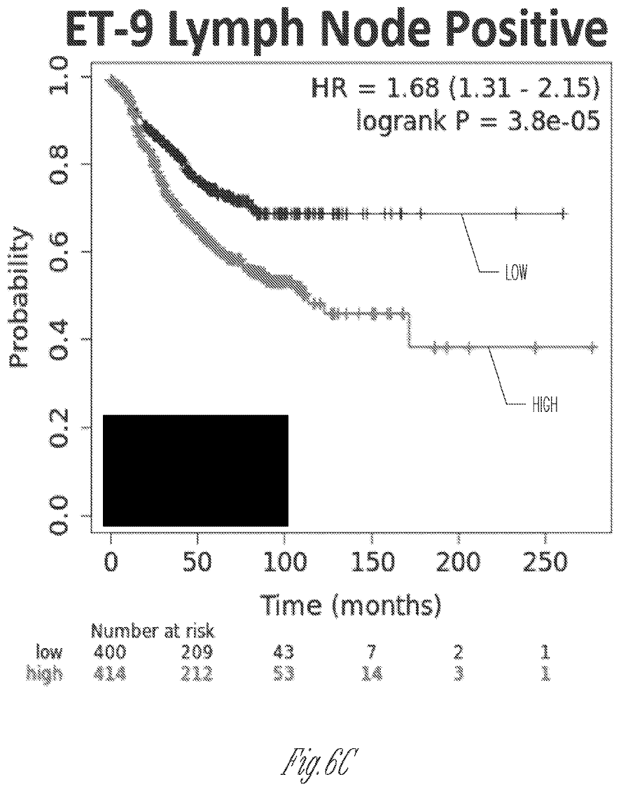

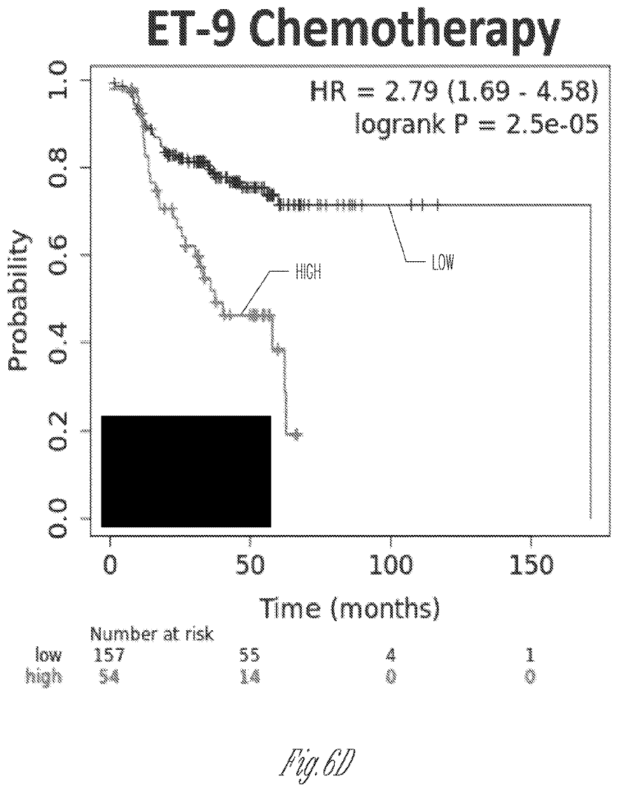

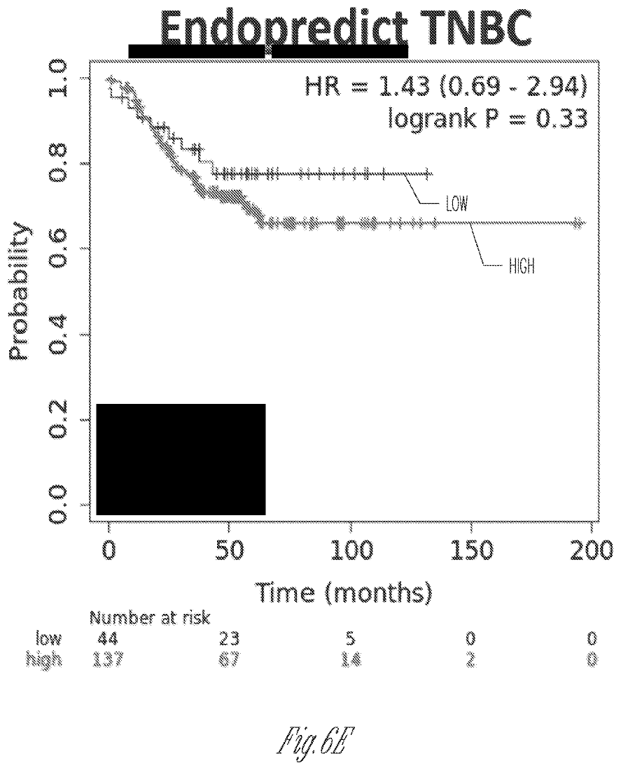

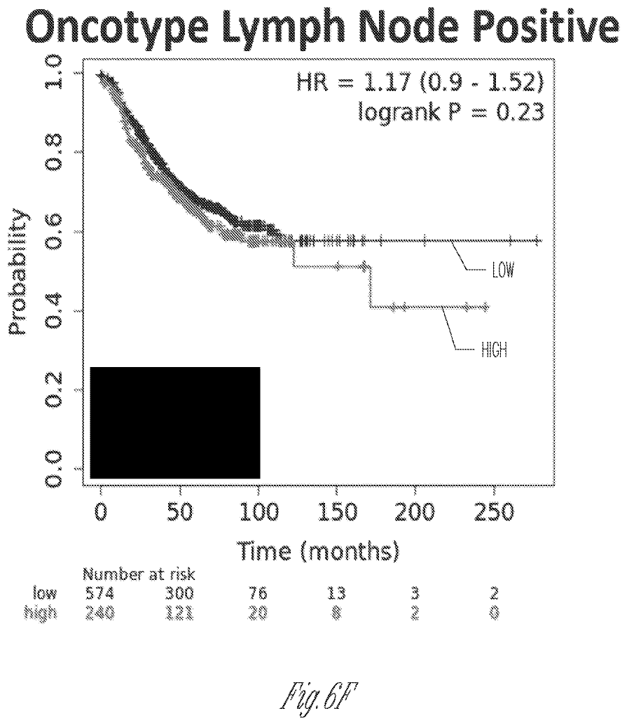

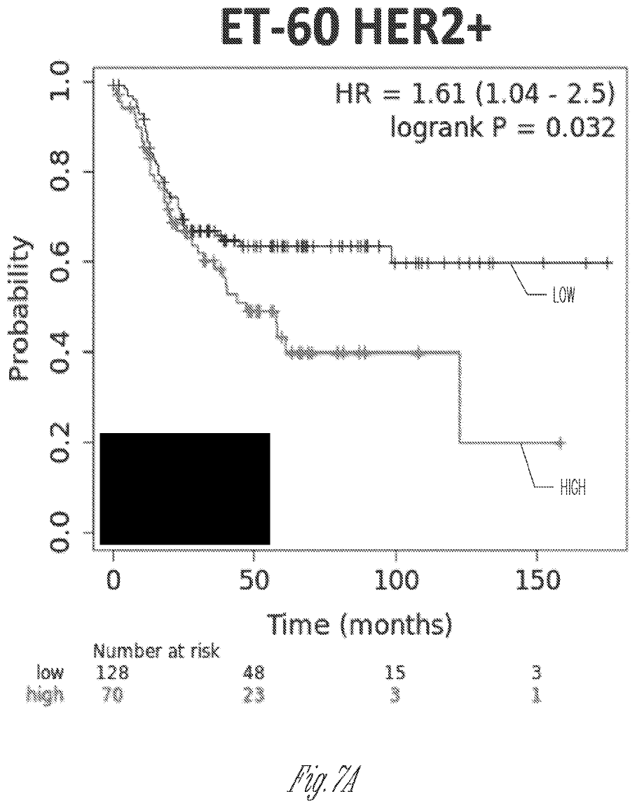

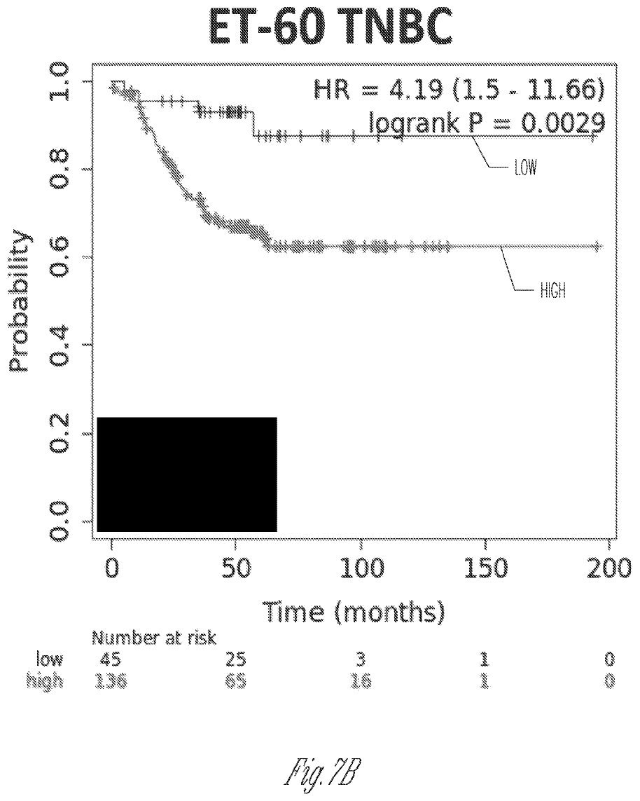

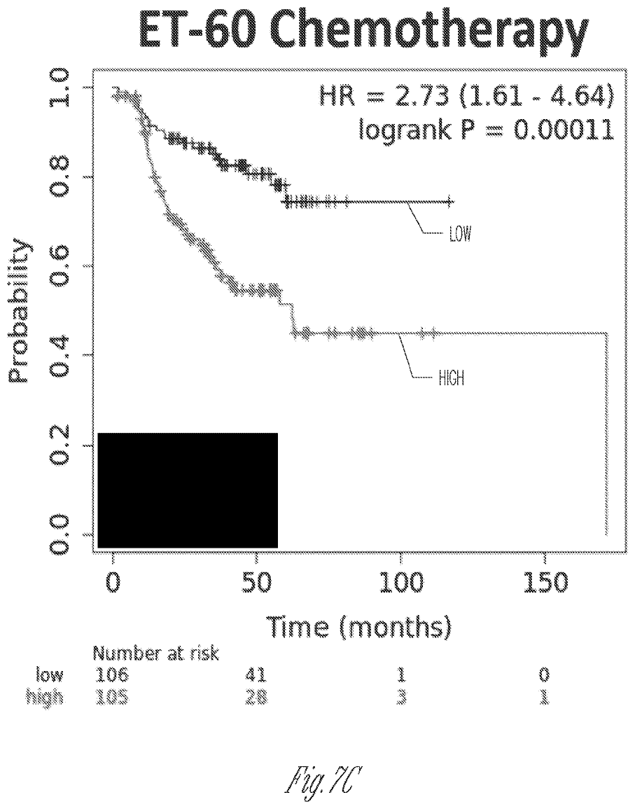

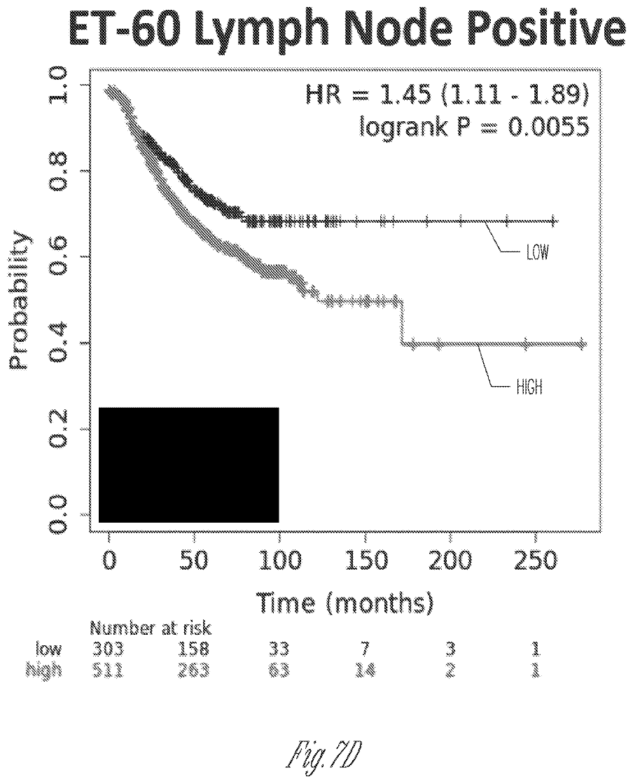

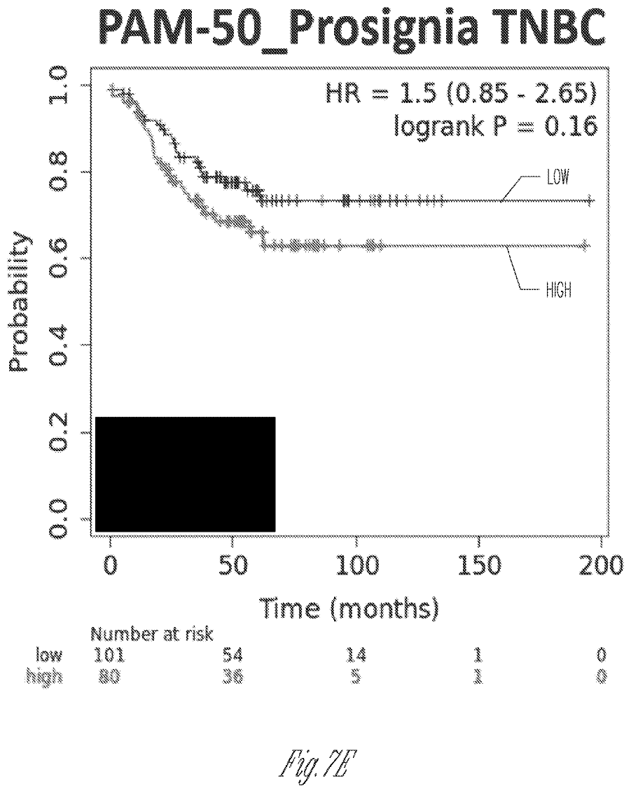

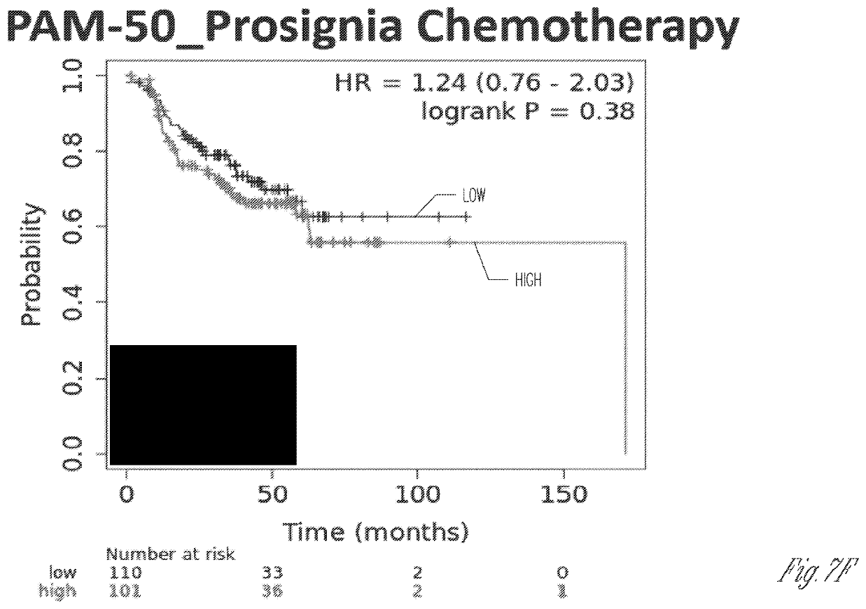

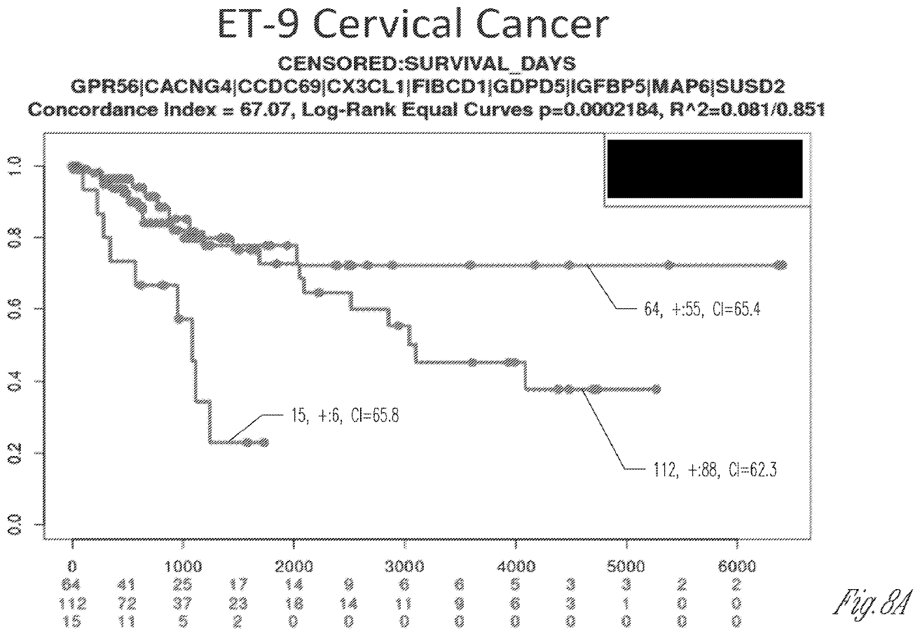

The inventors examined ET-60 and ET-9 in multiple combined breast datasets using K-M plotter (kmplot.com/analysis/) (Lanczky and Gyorffy; 23 (7), e27633, J Med Internet Res, 2021)] and have shown that ET-P and ET-60 signatures are predictive of worse survival outcome in other breast cancer subtypes such as HER-positive, ER-negative, Lymph Node positive, and post-chemotherapy breast cancers. These results indicate that ET-9 and ET-60 signatures do not overlap with existing commercial signatures and may have a broader and complimentary utility (FIG. 6E-6F and FIG. 7).

### Example 7: Other Cancer Types

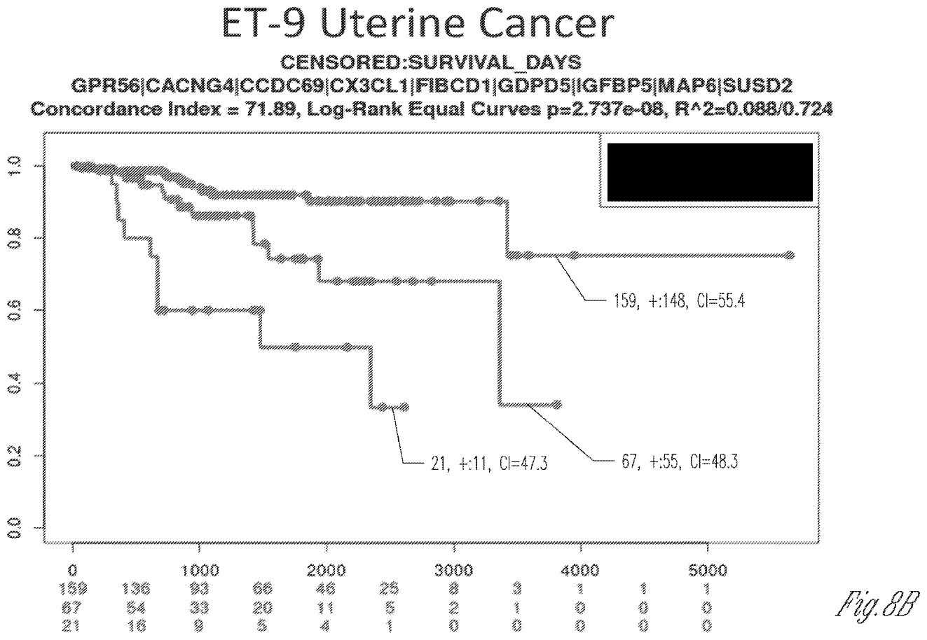

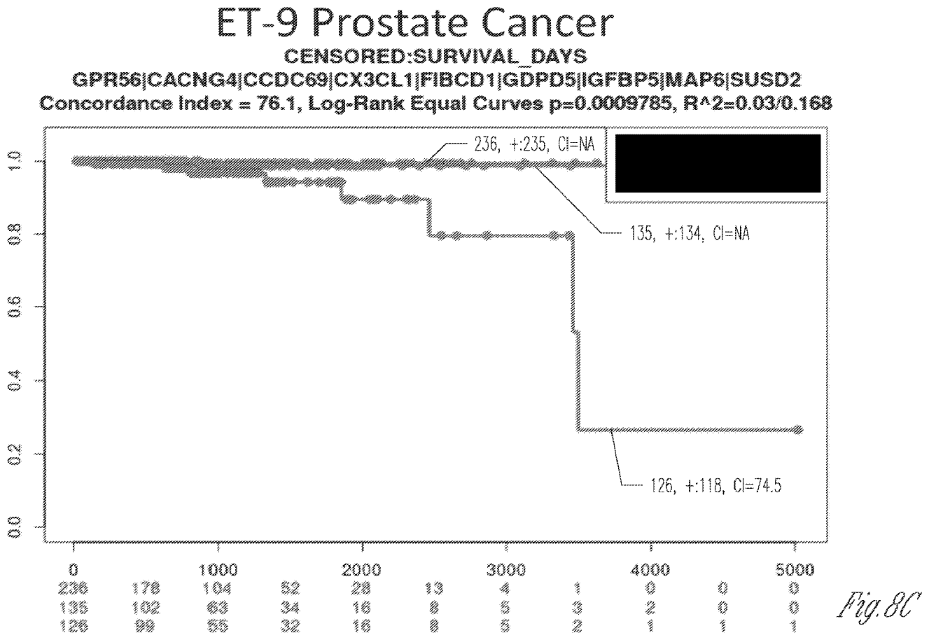

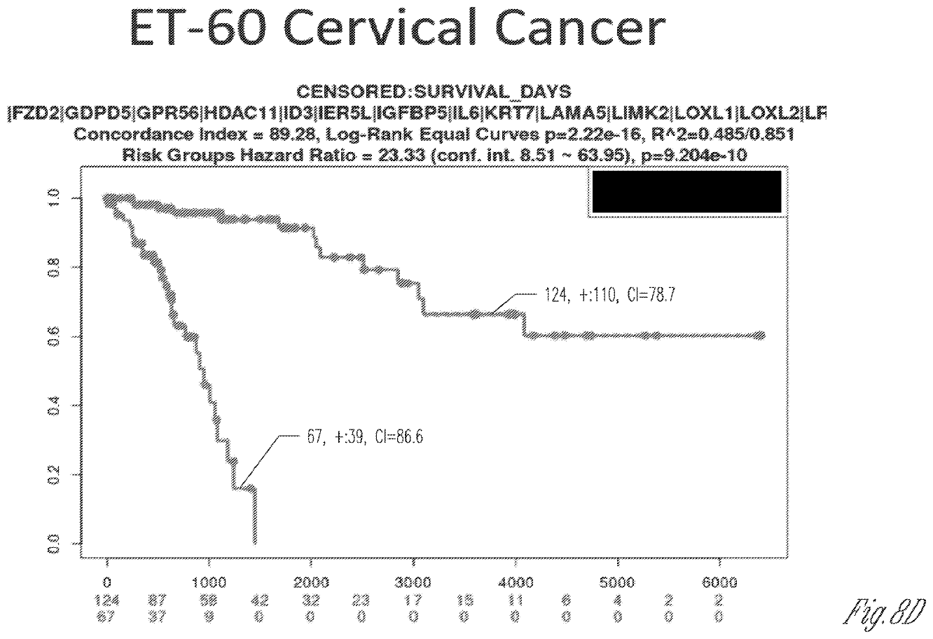

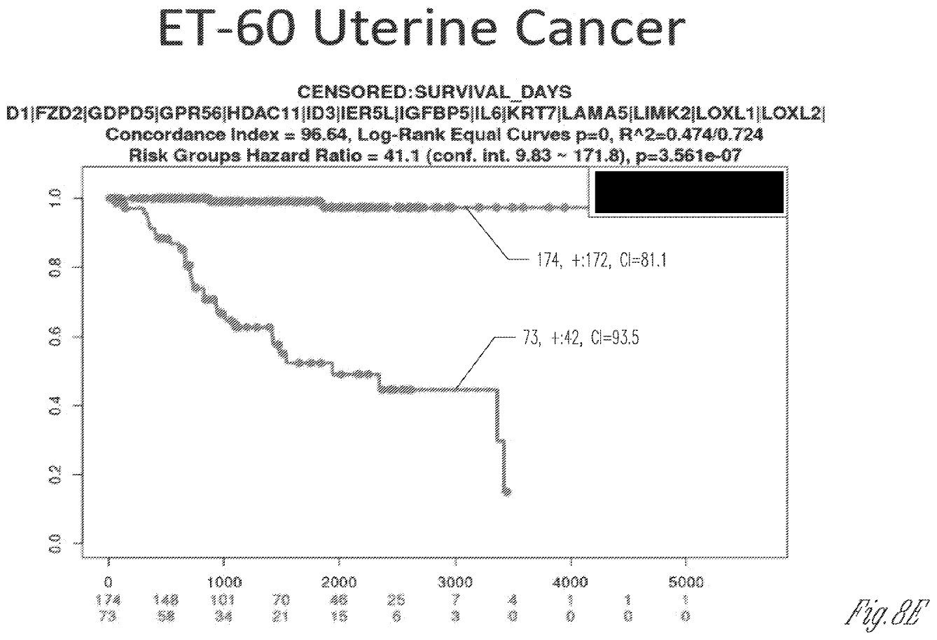

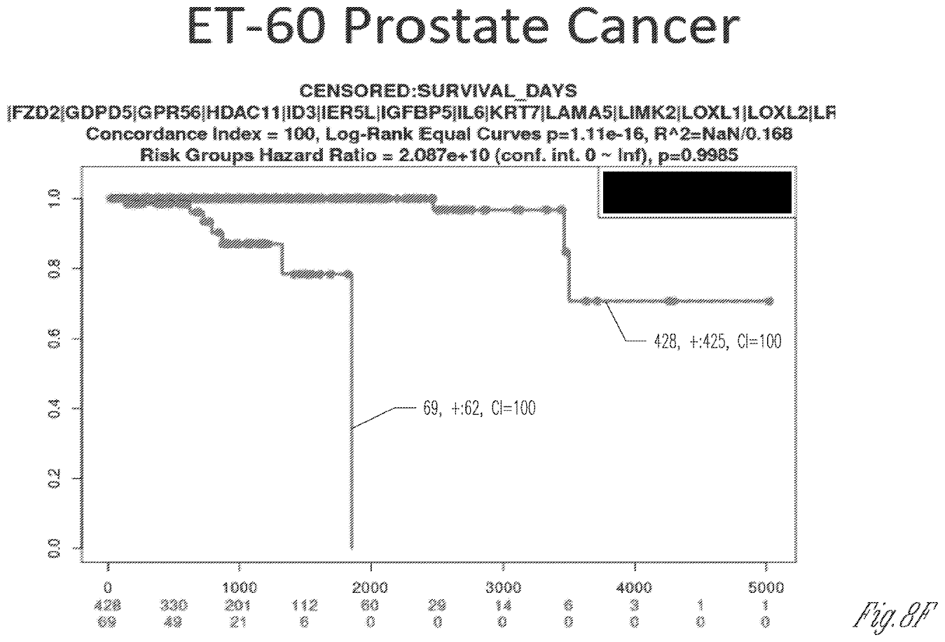

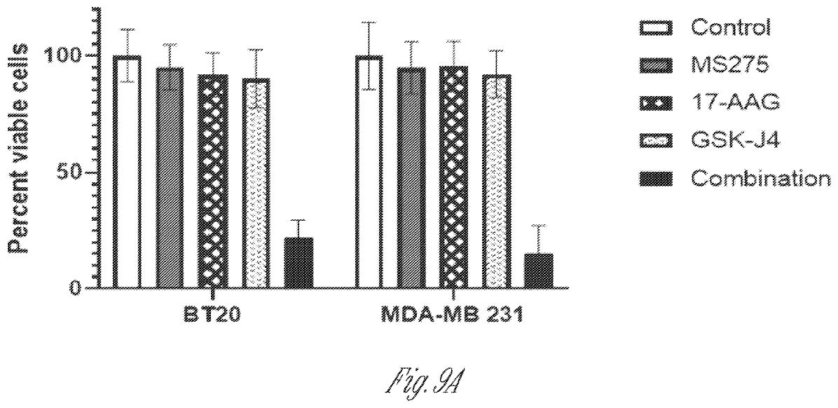

It was examined whether ET-60 or ET-9 signatures may be prognostic in other cancer types. As illustrated in FIG. 8, the ET-60 or ET-9 signatures do predict poor outcome in cervix, uterus and prostate cancers. These results illustrate that the utility of ET-9 and ET-60 signatures is not limited to breast cancer and may be prognostic in many cancer types.

### Example 8: Drug response

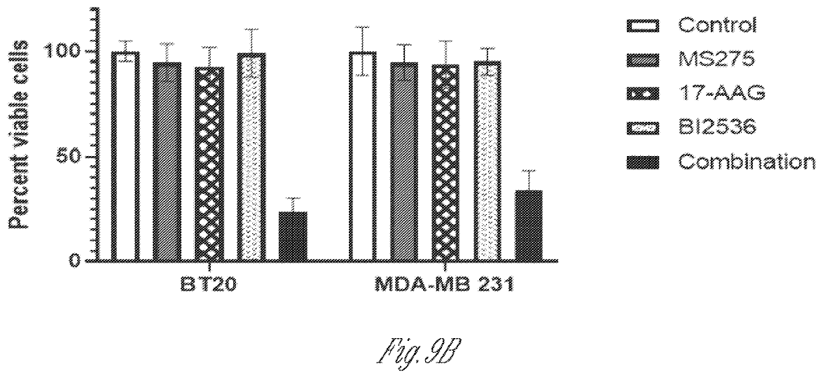

The breast cancer cell lines BT20, MDA-MB-231 and SUM-i 159 were treated with HDAC inhibitor (MS275), ISP inhibitor (17-AAG), mTOR inhibitor (Niclosamide), polo-like kinase inhibitor (1312536) and histone demethylase inhibitor (GSK-J4). As illustrated in FIG. 9, these results illustrate that the triple-drug combinations of these drugs synergistically inhibit breast cancer, which is a surprising result because the single treatments at the same dose are ineffective; the inhibition emerges only when the three drugs are combined.

Thus, the disclosure provides a pharmaceutical composition comprising two or more of a histone deacetylase inhibitor, a ZNF92 inhibitor, a histone demethylase inhibitor, a mTOR inhibitor, a polo-like kinase (PLK) inhibitor, or a heat shock factor inhibitor.

TABLE 3
Survival statistics of ET-9 signature in TCGA PanCancer Invasive Breast Cancer and METABRIC datasets

| | Patient Number | | | | | Median months survival (95% CI) | | | |
|---|---|---|---|---|---|---|---|---|---|
| Survival Type | Total | Altered | Events | Unaltered | Events | Altered | Unaltered | p-Value | q-Value |
| ET-9 TCGA | | | | | | | | | |
| Overall | 1084 | 379 | 67 | 705 | 84 | 112.08 | 216.75 | 1.64E−04 | 3.27E−04 |
| | | | | | | (100.70-NA) | (129.57-NA) | | |
| Progression | 1082 | 379 | 63 | 703 | 82 | 146.50 | NA | 2.31E−03 | 3.08E−03 |
| Free | | | | | | (113.82-NA) | | | |
| Disease- | 1063 | 371 | 44 | 692 | 39 | 113.82 | NA | 1.56E−05 | 6.23E−05 |
| specific | | | | | | (112.08-NA) | | | |
| Disease Free | 941 | 317 | 37 | 624 | 47 | NA | NA | 1.02E−02 | 1.02E−02 |
| ET-9 Metabric | | | | | | | | | |
| Overall | 1904 | 571 | 357 | 1333 | 746 | 131.30 | 164.60 | 5.07E−03 | 6.12E−03 |
| | | | | | | (119.00-154.00) | (152.07-175.97) | | |
| Relapse Free | 1903 | 571 | 253 | 1332 | 518 | 178.36 | 253.49 | 6.12E−03 | 6.12E−03 |
| | | | | | | (139.90-NA) | (203.85-NA) | | |

TABLE 4
Multivariate analysis of ET-9 signature in TCGA
PanCancer Invasive Breast Cancer datasets

| ET-9 non-significant clinical | Attribute | | p- | |
|---|---|---|---|---|
| associations (TCGA, PanCancer Atlas) | Type | Statistical Test | Value | q-Value |
| AJCC Disease Stage | Patient | Chi-squared Test | 0.349 | 0.509 |
| AJCC Lymph Node Stage | Patient | Chi-squared Test | 0.797 | 0.853 |
| AJCC Metastasis Stage | Patient | Chi-squared Test | 0.0623 | 0.145 |
| AJCC Tumor Stage | Patient | Chi-squared Test | 0.413 | 0.589 |
| Aneuploidy Score | Sample | Wilcoxon Test | 0.158 | 0.297 |
| Diagnosis Age | Patient | Wilcoxon Test | 0.515 | 0.64 |
| Ethnicity Category | Patient | Chi-squared Test | 0.335 | 0.496 |
| Fraction Genome Altered | Sample | Wilcoxon Test | 0.0347 | 0.111 |
| Mutation Count | Sample | Wilcoxon Test | 0.0121 | 0.0701 |
| Primary Lymph Node Presentation | Patient | Chi-squared Test | 0.424 | 0.589 |
| Assessment | | | | |
| Prior Diagnosis | Patient | Chi-squared Test | 0.0562 | 0.142 |
| Race Category | Patient | Chi-squared Test | 0.0205 | 0.0839 |
| Radiation Therapy | Patient | Chi-squared Test | 0.874 | 0.885 |
| Winter Hypoxia Score | Patient | Wilcoxon Test | 0.013 | 0.0701 |

TABLE 5
List of tumor types in the Human Protein Atlas PanCancer dataset

| | | No. of |
|---|---|---|
| | | samples |
| Cancer type | TCGA PanCancer Dataset | in TOGA |
| Breast cancer | Breast Invasive Carcinoma (BRCA) | 1075 |
| Cervical cancer | Cervical Squamous Cell Carcinoma and Endocervical | 291 |
| | Adenocarcinoma (CESC) | |
| Colorectal cancer | Colon Adenocarcinoma (COAD) | 438 |
| | Rectum Adenocarcinoma (READ) | 159 |
| Endometrial cancer | Uterine Corpus Endometrial Carcinoma (UCEC) | 541 |
| Glioma | Glioblastoma Multiforme (GBM) | 153 |
| Head and neck | Head and Neck Squamous Cell Carcinoma (HNSC) | 499 |
| cancer | | |
| Liver cancer | Liver Hepatocellular Carcinoma (LIHC) | 365 |
| Lung cancer | Lung Adenocarcinoma (LUAD) | 500 |
| | Lung Squamous Cell Carcinoma (LUSC) | 494 |
| Melanoma | Skin Cutaneous Melanoma (SKCM) | 102 |
| Ovarian cancer | Ovary Serous Cystadenocarcinoma (OV) | 373 |
| Pancreatic cancer | Pancreatic Adenocarcinoma (PAAD) | 176 |
| Prostate cancer | Prostate Adenocarcinoma (PRAD) | 494 |
| Renal cancer | Kidney Chromophobe (KICH) | 64 |
| | Kidney Renal Clear Cell Carcinoma (KIRC) | 528 |
| | Kidney Renal Papillary Cell Carcinoma (KIRP) | 285 |
| Stomach cancer | Stomach Adenocarcinoma (STAD) | 354 |
| Testis cancer | Testicular Germ Cell Tumor (TGCT) | 134 |
| Thyroid cancer | Thyroid Carcinoma (THCA) | 501 |
| Urothelial cancer | Bladder Urothelial Carcinoma (BLCA) | 406 |
| TOTAL | | 7932 |

TABLE 6
List of tumor types and samples in the TCGA PanCancer dataset

| | Study | |
|---|---|---|
| | Abbreviation | TCGA Study Name |
| 1 | ACC | Adrenocortical carcinoma |
| 2 | BLCA | Bladder Urothelial Carcinoma |
| 3 | BRCA | Breast invasive carcinoma |
| 4 | CESC | Cervical squamous cell carcinoma and |
| | | endocervical adenocarcinoma |
| 5 | CHOL | Cholangiocarcinoma |
| 6 | CNTL | Controls |
| 7 | COAD | Colon adenocarcinoma |
| 8 | DLBC | Lymphoid Neoplasm Diffuse Large B-cell Lymphoma |
| 9 | ESCA | Esophageal carcinoma |
| 10 | FPPP | FFPE Pilot Phase II |
| 11 | GBM | Glioblastoma multiforme |
| 12 | HNSC | Head and Neck squamous cell carcinoma |
| 13 | KICH | Kidney Chromophobe |
| 14 | KIRC | Kidney renal clear cell carcinoma |
| 15 | KIRP | Kidney renal papillary cell carcinoma |
| 16 | LAML | Acute Myeloid Leukemia |
| 17 | LCML | Chronic Myelogenous Leukemia |
| 18 | LGG | Brain Lower Grade Glioma |
| 19 | LIHC | Liver hepatocellular carcinoma |
| 20 | LUAD | Lung adenocarcinoma |
| 21 | LUSC | Lung squamous cell carcinoma |
| 22 | MESO | Mesothelioma |
| 23 | MISC | Miscellaneous |
| 24 | OV | Ovarian serous cystadenocarcinoma |
| 25 | PAAD | Pancreatic adenocarcinoma |
| 26 | PCPG | Pheochromocytoma and Paraganglioma |
| 27 | PRAD | Prostate adenocarcinoma |
| 28 | READ | Rectum adenocarcinoma |
| 29 | SARC | Sarcoma |
| 30 | SKCM | Skin Cutaneous Melanoma |
| 31 | STAD | Stomach adenocarcinoma |
| 32 | TGCT | Testicular Germ Cell Tumors |
| 33 | THCA | Thyroid carcinoma |
| 34 | THYM | Thymoma |
| 35 | UCEC | Uterine Corpus Endometrial Carcinoma |
| 36 | UCS | Uterine Carcinosarcoma |
| 37 | UVM | Uveal Melanoma |

TABLE 7A
List of breast cancer molecular signatures tested in cBioPortal for Cancer Genomics survival analysis (cbioportal.org/)

| Oncogene Pathways | Signature Tested |
|---|---|
| ET-9 Signature (9 genes) | ADGRG1 (GPR56), CACNG4, CCDC69, CX3CL1, FIBCD1, GDPD5, IGFBP5, MAP6, |
| | SUSD2 |
| Cell Cycle (34 genes) | RB1 RBL1 RBL2 CCNA1 CCNB1 CDK1 CCNE1 CDK2 CDC25A COND1 CDK4 CDK6 |
| | CCND2 CDKN2A CDKN2B MYC CDKN1A CDKN18 E2F1 E2F2 E2F3 E2F4 E2F5 E2F6 |
| | E2F7 E2F8 SRC JAK1 JAK2 STAT1 STAT2 STAT3 STAT5A STATSB |
| P53 (6 genes) | TP53 MDM2 MDM4 CDKN2A CDKN2B TP53BP1 |
| PI3K-AKT-mTOR signaling (17 | PIK3CA PIK3R1 PIK3R2 PTEN PDPK1 AKT1 AKT2 FOXO1 FOXOB MTOR RICTOR TSC1 |
| genes) | TSC2 RHEB AKT1S1 RPTOR MLST8 |
| Notch Signaling (55 genes) | ADAM10 ADAM17 APH1A APH1B ARRDC1 CIR1 CTBP1 CTBP2 CUL1 DLL1 DLL3 DLL4 |
| | DTX1 DTX2 DTX3 DTX3L DTX4 EP300 FBXW7 HDAC1 HDAC2 HES1 HES5 HEYL ITCH |
| | JAG1 JAG2 KDM5A LFNG MAML1 MAML2 MAML3 MENG NCOR2 NCSTN NOTCH1 |
| | NOTCH2 NOTCH3 NOTCH4 NRARP NUMB NUMBL PSEN1 PSEN2 PSENEN RBPJ |
| | RBPIL RENG SNW1 SPEN HESZ HES4 HES7 HEY1 HEY2 |
| Ras-Raf-MEK-Erk/INK signaling | KRAS HRAS BRAF RAF1 MAP3K1 MAP3K2 MAP3K3 MAP3K4 MAP3K5 MAP2K1 |
| (26 genes) | MAP2K2 MAP2K3 MAP2K4 MAP2K5 MAPK1 MAPK3 MAPK4 MAPK6 MAPK7 MAPK& |
| | MAPK9 MAPK12 MAPK14 DAB2 RASSF1 RAB25 |
| TGF-B Pathway (43 genes) | TGFB1 TGFB2 TGFB3 TGFBR1 TGFBR2 TGFBR3 BMP2 BMP3 BMP4 BMP5 BMP6 |
| | BMP7 GDF2 BMP10 BMP15 BMPR1A BMPR1B BMPR2 ACVR1 ACVR1B ACVR1C |
| | ACVR2A ACVR2B ACVRL1 Nodal GDF1 GDF11 INHA INHBA INHBB INHBC INHBE |
| | SMAD2 SMAD3 SMAD1 SMAD5 SMAD4 SMAD9 SMAD6 SMAD7 SPTBN1 TGFBRAP1 |
| | ZFYVE9 |
| Oncotype Dx | CTSV, GRB7, ERBB2, ESR1, PGR, BCL2, SCUBE2, GSTM1, BAG1, CD68, ACTB, GAPDH, |
| | GUS, RPLPO, TFRC |
| Mammaprint | ESM1, IGFBP5, FGF18, SCUBE2, TGFB3, WISP1,FLT1, HRASLS, STK32B, RASSF7, DCK, |
| | MELK, EXT1, GNAZ, EBF4, MTDH, PITRM1, QSCN6L1, BBC3, EGLN1, TGFB3, ESM1, |
| | IGFBP5, FGF18, SCUBE2, TGFB3, WISP1, FLT1, HRASLS, STK32B, RASSF7, DCK, |
| | MELK, EXT1, GNAZ, EBF4, MTDH, PITRM1, QSCN6L1, CCNE2, ECT2, CENPA, LIN9, |
| | KNTC2, MCM6, NUSAP1, ORC6L, TSPYL5, RUNDC1, PRC1, RFC4, RECQL5, CDCA7, |
| | DTL, COL4A2, GPR180, MMP9, GPR126, RTN4RL1, DIAPH3, CDC42BPA, PALM2, |
| | TGFB3, IGFBP5, FGF18, WISP1, ALDH4 A1, AYTL2, OXCT1, PECI, GMPS, GSTM3, |
| | SLC2 A3, FLT1, FGF18, COL4 A2, GPR180, EGLN1, MMP9 |
| 9 gene prognostic signature | TCAP., STARD3, CDR2L, PNMT, GPR4, ANGPT2, CAPN5, STXBP3, PKN2 |

TABLE 7B
Survival statistics of breast cancer molecular signatures tested in
cBioPortal for Cancer Genomics survival analysis (cbioportal.org/)

| | | TCGA PanCancer Atlas, Breast invasive | | | | | |
|---|---|---|---|---|---|---|---|
| | — | carcinoma (n = 1,084) | | | | | |
| | Altered | | Progression | Disease- | Disease | METABRIC (n = 1,904) | |
| | % | Overall | free | specific | Free | Overall | Relapse Free |
| ET-9 Signature (9 genes) | 30-35% | 1.64E−04 | 2.31E−03 | 1.56E−05 | 1.02E−02 | 5.07E−03 | 6.12E−03 |
| Cell Cycle Control (34 genes) | 72% | p = 0.26 | p = 0.26 | p = 0.55 | 3.80E−02 | p = 0.30 | p = 0.21 |
| p53 (6 genes) | 26-40% | p = 0.77 | P = 0.85 | p = 0.66 | P = 0.90 | p = 0.74 | 1.08E−02 |
| PI3K-AKT-mTOR signaling (17 | 62-70% | p = 0.29 | p = 0.29 | p = 0.47 | p = 0.61 | p = 0.059 | p = 0.45 |
| genes) | | | | | | | |
| Notch Signaling (55 genes) | 86-92% | p = 0.79 | p = 0.46 | p = 0.18 | p = 0.31 | p = 0.95 | p = 0.45 |
| Ras-Raf-MEK-Erk/JNK | 68-79% | p = 0.10 | p = 0.60 | p = 0.25 | p = 0.63 | p = 0.07 | p = 0.26 |
| signaling (26 genes) | | | | | | | |
| TGF-B Pathway (43 genes) | 73-74% | p = 0.36 | p = 0.22 | p = 0.63 | p = 0.19 | p = 0.35 | p = 0.27 |
| Oncotype Dx (21 genes) | 53-62% | p = 0.66 | p = 0.97 | p = 0.88 | p = 0.86 | p = 0.09 | 3.25E−03 |
| Mammaprint | 86-90% | p = 0.09 | p = 0.20 | 3.03E−02 | p = 0.19 | 9.68E−03 | p = 0.47 |
| 9-gene signature | 37-39% | p = 0.64 | p = 0.71 | p = 0.56 | p = 0.80 | 0.0156 | 1.14E−04 |

### REFERENCES

Aguirre-Gamboa, R, Conez-Rueda, -I., Martinez-Ledesma, E., Martinez-Torteya, A, Chacolla-Huaringa, R., Rodriguez-Barrientos, A., Tamez-Pena, J.G., Trevino, V., 2013. SurvExpress: an online biomarker validation tool and database for cancer gene expression data using survival analysis. PLoS One 8, e74250.
Bartha, A., Gyorffy, B., 2021. TNMplot.com: A Web Tool for the Comparison of Gene Expression in Normal, Tumor and Metastatic Tissues. Int J Mol Sci 22.
Gao, J., Aksoy, B. A., Dogrusoz, U., Dresdner, G., Gross, B., Sumer, S. O., Sun, Y., Jacobsen, A., Sinha, R., Larsson, E., Cerami, E., Sander, C., Schultz, N., 2013.
Integrative analysis of complex cancer genomics and clinical profiles using the cBioPortal. Sci Signal 6, pl1.
Giuliano, A.E., Connolly, J. L., Edge, S. B., Mittendorf, E. A., Rugo, H. S., Solin, L. J., Weaver, D. L., Winchester, D. J, J-ortobagyi, G N, 2017. Breast Cancer-Major changes in the American Joint Committee on Cancer eighth edition cancer staging manual. C A Cancer J Clin 67, 290-303.
Johansson, A L.V., Trewin, C.B., Fredriksson, I., Reinertsen, K. V., Russnes, H., Jrsin, G., 2021. In modem times, how important are breast cancer stage, grade and receptor subtype for survival: a population-based cohort study. Breast Cancer Res 23, 17.
Lanczky, A., Gyorffy, B., 2021. Web-Based Survival Analysis Tool Tailored for Medical Research (K.Mplot): Development and Implementation. J Med Internet Res 23, e27633.
Lee, U., Frankenberger, C., Yun, J., Bevilacqua, E., Caldas, C., Chin, S. F, Rueda, OM., Reinitz, J., Rosner, M. R., 2013. A prognostic gene signature for metastasis-free survival oftriple negative breast cancer patients. PLoS One 8, e82125.
Nunes, A. T., Collyar, D. E., Harris, L. N., 2017. Gene Expression Assays for Early-Stage Hormone Receptor-Positive Breast Cancer: Understanding the Differences. JNCI Cancer Spectr 1, pkx008.
Park, S., Yoon, B. H., Kim, S.K., Kim, S. Y., 2019. GENT2: an updated gene expression database for normal and tumor tissues. BMC Med Genomics 12, 101.
Ponten, F., Schwenk, J. M., Asplund, A., Edqvist, PH, 2011L The Human Protein Atlas as a proteomic resource for biomarker discovery. J Intern Med 270, 428-446.
Saadatmand, S., Bretveld, R., Siesling, S., Tilanus-Linthorst, M. M., 2015. Influence of tumour stage at breast cancer detection on survival in modern times: population based study in 173,797 patients. BMJ 351, h4901.
Sole, X., Bonifaci, N., Lopez-Bigas, N., Berenguer, A., Hernandez, P., Reina, O., Maxwell, C A., Aguilar, H., Urruticoechea, A, de Sanjose, S., Comnellas, F., Capella, G., Moreno, V., Pujana, M. A., 2009. Biological convergence of cancer signatures. PLoS One 4, e4544.
Sotiriou, C., Pusztai, L., 2009. Gene-expression signatures in breast cancer. N Engl J Med 360, 790-800.
Stormo, C., Kringen, M. K., Lyle, R., Oistad, O. K., Sachse, D., Berg, J. P., Piehler, A. P. 2014. RNA-sequencing analysis ofHepG-2 cells treated with atorvastatin. PLoS One 9, e105836.
Venet, D., Dumont, J. E., Detours, V., 2011. Most random gene expression signatures are significantly associated with breast cancer outcome. PLoS Comput Biol 7, e1002240.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various features of the invention according to the foregoing description provided in the specification and figures.

### Statements:

1. A method comprising:
   a. assaying a biological sample from a subject for expression of ZNF92, ET-9 biomarkers recited in Table 1, or nine or more of the ET-60 biomarkers recited in Table 2 to determine one or more expression levels for the ZNF92, ET-9, or nine or more of the ET-60 biomarkers;
   b. comparing the determined expression levels with one or more reference values to identify any altered expression levels in the subject's biological sample, wherein altered expression levels of the ZNF92, ET-9, or nine or more of the ET-60 biomarkers in the biological sample relative to the reference value indicates that the subject has cancer with poor prognosis or the subject has malignant cancer, and absence of altered expression of the ZNF92, ET-9, or nine or more of the ET-60 biomarkers relative to the reference value indicates that the subject does not have a cancer with poor prognosis or does not have malignant cancer; and optionally
   c. administering one or more histone deacetylase inhibitors, ZNF92 inhibitors, histone demethylase inhibitors, mTOR inhibitors, polo-like kinase (PLK) inhibitors, heat shock factor inhibitors, or a combination thereof to a subject determined to have a cancer with poor prognosis or a malignant cancer.
2. A method of treating a subject classified as having poor cancer prognosis, comprising administering one or more histone deacetylase inhibitors, ZNF92 inhibitors, histone demethylase inhibitors, mTOR inhibitors, polo-like kinase inhibitors, heat shock factor inhibitors, or a combination thereof to the subject, wherein the subject is classified has having poor cancer prognosis by measuring expression levels of at least one sample from the subject and determining that the at least one sample has altered expression of the ZNF92, ET-9, or nine or more of the ET-60 biomarkers relative to at least one reference value.
3. A method, comprising treating a subject having altered expression of ZNF92, ET-9 biomarkers, or nine or more of the ET-60 biomarkers relative to at least one reference value, by administering one or more histone deacetylase inhibitors, ZNF92 inhibitors, histone demethylase inhibitors, mTOR inhibitors, polo-like kinase inhibitors, heat shock factor inhibitors, or a combination thereof to the subject.
4. The method of statement 1, 2 or 3, wherein the one or more reference values is an average or median of expression levels of at least the ZNF92, ET-9, or ET-60 biomarkers in biological samples from a population of healthy subjects.
5. The method of statement 1-3, or 4, wherein the subject has, or is suspected of having, breast cancer, ovarian cancer, colon cancer, brain cancer, pancreatic cancer, prostate cancer, lung cancer, melanoma, leukemia, mycloma, or lymphoma.
6. The method of statement 1-4, or 5, wherein the subject has breast cancer.
7. The method of statement 1-5 or 6, wherein the altered expression of one or more of the ZNF92, ET-9, or nine or more of the ET-60 biomarkers is increased expression relative to the reference value.
8. The method of statement 1-5 or 6, wherein the altered expression of one or more of the ZNF92, ET-9, or nine or more of the ET-60 biomarkers is decreased expression relative to the reference value.
9. The method of statement 1-7 or 8, wherein the altered expression of the ZNF92, ET-9, or nine or more of the ET-60 biomarkers relative to the reference value is a difference of at least 10% as compared to a reference level, or of at least about 20%, or at least about 30%, or at least about 40%, or at least about 50%, or at least about 60% or at least about 70%, or at least about 80%, or at least about 90% or up to and including a 100% increase or any increase between 10-100% as compared to a reference value, or at least about 1.5-fold, at least about a 1.6-fold, at least about a 1.7-fold, at least about a 1.8-fold, at least about a 1.9-fold, at least about a 2-fold, at least about a 3-fold, or at least about a 4-fold, or at least about a 5-fold, at least about a 10 fold compared to the reference value.
10. A method comprising: (a) contacting ZNF92-expressing cells or ZNF92 proteins with a test agent; (b) measuring ZNF92 expression (mRNA or protein) levels in the cells or measuring ZNF92 protein activity levels; and (c) determining that the test agent reduces the expression levels or activity levels of ZNF92, to thereby identifying a test agent as a candidate agent that reduces ZNF92 expression levels or activity levels.
11 A method comprising: (a) contacting cells that expression one or more ET-9 or ET-60 biomarkers with a test agent; (b) measuring expression (mRNA or protein) levels or measuring activity levels of the one or more ET-9 or ET-60 biomarkers; and (c) determining that the test agent reduces the expression levels or activity levels of the one or more ET-9 or ET-60 biomarkers, to thereby identifying a test agent as a candidate agent that reduces one or more ET-9 or ET-60 biomarkers expression levels or activity levels.

The specific methods, devices and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising:
a. assaying a biological sample from a subject for expression of ZNF92, of two or more ET-9 biomarkers recited in Table 1, or nine or more of the ET-60 biomarkers recited in Table 2, or a combination thereof, to determine one or more expression levels for the ZNF92, two or more of ET-9, or nine or more of the ET-60 biomarkers, or a combination thereof,
b. comparing the determined expression levels with one or more reference values to identify any altered expression levels in the subject's biological sample, wherein altered expression levels of the ZNF92, ET-9, or nine or more of the ET-60 biomarkers in the biological sample relative to the reference value indicates that the subject has cancer with poor prognosis or the subject has malignant cancer, and absence of altered expression of the ZNF92, ET-9, or nine or more of the ET-60 biomarkers relative to the reference value indicates that the subject does not have a cancer with poor prognosis or does not have malignant cancer; and optionally
c. administering one or more histone deacetylase inhibitors, ZNF92 inhibitors, histone demethylase inhibitors, mTOR inhibitors, polo-like kinase (PLK) inhibitors, heat shock factor inhibitors, or a combination thereof, to a subject determined to have a cancer with poor prognosis or a malignant cancer.

2. The method of claim 1 wherein the sample is a breast cancer sample.

3. The method of claim 1 wherein the sample is a cervical cancer sample.

4. The method of claim 1 wherein the sample is a uterine cancer sample.

5. The method of claim 1 wherein the sample is a prostate cancer sample.

6. The method of claim 1 wherein the sample is a physiological fluid sample.

7. The method of claim 1 wherein the subject is a human.

8. The method of claim 1 wherein expression of ZNF92 is assayed.

9. The method of claim 1 wherein expression of three, four or five of ET-9 biomarkers are assayed.

10. The method of claim 1 wherein expression of ten, eleven, twelve or twenty of ET-60 biomarkers are assayed.

11. The method of claim 1 wherein RNA expression is assayed.

12. The method of claim 11 wherein nucleic acid amplification is employed prior to assaying.

13. The method of claim 1 wherein protein expression is assayed.

14. A method to prevent, inhibit or treat cancer in a mammal, comprising: administering to the mammal a composition comprising one or more histone deacetylase inhibitors, ZNF92 inhibitors, histone demethylase inhibitors, mTOR inhibitors, polo-like kinase (PLK) inhibitors, heat shock factor inhibitors, or a combination thereof, wherein the mammal determined to have altered expression levels of ZNF92, two or more ET-9 biomarkers, or nine or more of the ET-60 biomarkers, or a combination thereof, relative to a reference value.

15. The method of claim 14 wherein the mammal is a human.

16. The method of claim 14 wherein the mammal has breast cancer.

17. The method of claim 14 wherein the mammal has cervical cancer.

18. The method of claim 14 wherein the mammal has uterine cancer.

19. The method of claim 14 wherein the mammal has prostate cancer.

20. A method comprising: (a) contacting ZNF92-expressing cells or ZNF92 proteins with a test agent; (b) measuring ZNF92 RNA or protein expression levels in the cells or measuring ZNF92 protein activity levels; and (c) determining that the test agent reduces the expression levels or activity levels of ZNF92, to thereby identifying a test agent as a candidate agent that reduces ZNF92 expression levels or activity levels.

21. A method comprising: (a) contacting cells that expression one or more ET-9 or ET-60 biomarkers with a test agent; (b) measuring expression RNA or protein levels or measuring activity levels of the one or more ET-9 or ET-60 biomarkers; and (c) determining that the test agent reduces the expression levels or activity levels of the one or more ET-9 or ET-60 biomarkers, to thereby identifying a test agent as a candidate agent that reduces one or more ET-9 or ET-60 biomarkers expression levels or activity levels.

22. A pharmaceutical composition comprising two or more of a histone deacetylase inhibitor, a ZNF92 inhibitor, a histone demethylase inhibitor, a mTOR inhibitor, a polo-like kinase (PLK) inhibitor, or a heat shock factor inhibitor.


SEQUENCE LISTING (71 sequences)

SEQ ID NO: 1 - protein, 586 aa, Homo sapiens
MGPLTFRDVKIEFSLEEWQCLDTAQRNLYRDVMLENYRNLVFLGIAVSKPDLITWLEQGKEPWNLKRHEMVDKTPVMCSHFAQDVWPEHSIKDSFQKVILRTYGKYGHENLQLRKDHKSVDACKVYKGGYNGLNQCLTTTDSKIFQCDKYVKVFHKFPNVNRNKIRHTGKKPFKCKNRGKSFCMLSQLTQHKKIHTREYSYKCEECGKAFNWSSTLTKHKIIHTGEKPYKCEECGKAFNRSSNLTKHKIIHTGEKPYKCEECGKAFNRSSTLTKHKRIHTEEKPYKCEECGKAFNQFSILNKHKRIHMEDKPYKCEECGKAFRVFSILKKHKIIHTGEKPYKCEECGKAFNQFSNLTKHKIIHTGEKPYKCDECGKAFNQSSTLTKHKRIHTGEKPYKCEECGKAFKQSSTLTEHKIIHTGEKPYKCEKCGKAFSWSSAFTKHKRNHMEDKPYKCEECGKAFSVFSTLTKHKIIHTREKPYKCEECGKAFNQSSIFTKHKIIHTEGKSYKCEKCGNAFNQSSNLTARKIIYTGEKPYKYEECDKAFNKFSTLITHQIIYTGEKPCKHECGRAFNKSSNYTKEKLQT

SEQ ID NO: 2 - DNA, 3123 bp, Homo sapiens
  source 1..3123: mol_type = other DNA
ctctcgctgcagccggcgctccacgtctagtcttcactgctctgcgtcctgtgctgataaaggctcgccgctgtgaccctgttacctgcaagaacttggaggttcacagctaagacgccaggaccccctggaagcctagaaatgggaccactgacatttagggatgtgaaaatagaattctctctagaggaatggcaatgcctggacactgcgcagcggaatttatatagagatgtgatgttagagaactacagaaacctggtcttccttggtattgctgtctctaagccagacctgatcacctggctggagcaaggaaaagagccctggaatctgaagagacatgagatggtagacaaaaccccagttatgtgttctcattttgcccaagatgtttggccagagcacagcataaaagattctttccaaaaagtgatactgagaacatatggaaaatatggacatgagaatttacagctaagaaaagaccataaaagtgtggatgcatgtaaggtgtacaaaggaggttataatggacttaaccagtgtttgacaactactgacagcaagatatttcagtgtgataaatatgtgaaagtctttcataaatttccaaatgtaaatagaaataagataagacatactggaaagaaacctttcaaatgtaaaaaccgtggcaaatcattttgcatgctttcacaattaactcaacataagaaaattcatactagagagtattcttacaaatgtgaagaatgtggtaaagcctttaactggtcctcaacccttactaaacataagataattcatactggagaaaaaccctacaaatgtgaagaatgtggcaaagcttttaaccggtcctcaaatcttactaaacataaaataattcatactggagagaaaccctacaaatgtgaagaatgtggcaaagcttttaaccggtcctcaacccttactaaacataaaagaattcatacagaagagaaaccctacaaatgtgaagaatgtggcaaggcctttaaccagttctcgattcttaataaacataagagaattcatatggaagataaaccctacaaatgtgaagaatgtggcaaagcctttagagtattctcaattcttaaaaaacataagataatccatactggggaaaaaccatacaaatgtgaagaatgtggcaaagcctttaaccagttctcaaaccttactaaacataagataattcatactggagagaaaccctacaaatgtgatgaatgtggcaaagcctttaaccagtcctcaacccttactaaacataaaagaattcatacgggagaaaaaccctacaaatgtgaagaatgtggcaaagcttttaaacagtcctcaacccttactgaacataagataattcatactggagagaaaccctacaaatgtgaaaaatgtggcaaggcctttagctggtcctcagcttttactaaacataagagaaatcatatggaagataaaccctacaaatgtgaagaatgtggcaaagcctttagtgtattctcaacccttactaaacataaaataattcatactagagaaaaaccctacaaatgtgaagaatgtggcaaagcctttaaccagtcctcaatttttactaaacataagataattcacactgaagggaaatcctacaaatgtgaaaaatgtggcaatgcttttaaccagtcctcaaaccttactgcacgtaagataatttatactggagagaaaccctacaaatatgaagaatgtgacaaagcctttaacaagttctcaacccttattacacatcagataatttatactggagagaaaccctgcaaacatgaatgtggcagagcctttaacaaatcctcaaattatactaaagagaaactacaaacctgaaagatgtgacaatgattttcactacacctcaaacttttctaaacataaaccatattggtgccctagaaatgtgaggaatatgacaaggactttaaatggttgtcacgcttgattgtaggtaagataatttatattggagaaaaatcctccaagtatgaagaatgtggcaaacttttaaccaatcctcacaccttattgcacaggaaagcatttatacttgagaaaaattgtataaagaatatggaaaagccatttatatctgctcacatgtaaaaacatcagttcatacttaataaaatgcaattaccgtcaaatctttcagaaaatataagcctttaatacgaggaagagtattcttaagatgaacattacaaatagaaagagggttgtagtacctttagttttatgatagatcttattgtacacattttgtaccagaggaaaaccctaaagcattagttgctcaaactttgttcgacatcagggaatttgtattggagaaaaaccctgcaaatgtaataaatatggaaaaacattttttcaaaaactacagcttggaaaacatcagagagttcatactaaaatatatttttgcagatgcagtaaatatgaaaaatatttaatcccaaattaagtctatgtaaatatcagaattcacagtagaaatcataaggcataaggcactgatacttcagacattacactaaattagagtgttgagtataggagatccaaaactaaaattgttaggtaagttatttatatataactttaaaagaagtagaagattttttggagatttataattacattcaaagtatacttttttcttgaaaaaaattacagattttttgaaaagcaattgatgtaatttaactctcaaattcatgtttttcttcattcctattatattcacatgtgaaagcaagtgatctgttgttgctgaatcagagatatgagagattcttttttataggtgggcattatttatgcccctttctgtggaagagtaagaaaattaaaatacaagatgcatgaggaaaatgtagagatgctctttgtgattaacttagaatattaagtgctacttgacgtacatgttcagactaacattcttttgcagtatagtgagaaaaaaacattttaaaattaattatcattttgttgattgtgcttttatgtaataaaatgcagtactttaaaacaaaaaaaaaaaaaaaa

SEQ ID NO: 3 - protein, 693 aa, Homo sapiens
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 4 - protein, 461 aa, Homo sapiens
MVNDRWKTMGGAAQLEDRPRDKPQRPSCGYVLCTVLLALAVLLAVAVTGAVLFLNHAHAPGTAPPPVVSTGAASANSALVTVERADSSHLSILIDPRCPDLTDSFARLESAQASVLQALTEHQAQPRLVGDQEQELLDTLADQLPRLLARASELQTECMGLRKGHGTLGQGLSALQSEQGRLIQLLSESQGHMAHLVNSVSDILDALQRDRGLGRPRNKADLQRAPARGTRPRGCATGSRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPVREDR

SEQ ID NO: 5 - protein, 605 aa, Homo sapiens
MVRHQPLQYYEPQLCLSCLTGIYGCRWKRYQRSHDDTTPWERLWFLLLTFTFGLTLTWLYFWWEVHNDYDEFNWYLYNRMGYWSDWPVPILVTTAAAFAYIAGLLVLALCHIAVGQQMNLHWLHKIGLVVILASTVVAMSAVAQLWEDEWEVLLISLQGTAPFLHVGAVAAVTMLSWIVAGQFARAERTSSQVTILCTFFTVVFALYLAPLTISSPCIMEKKDLGPKPALIGHRGAPMLAPEHTLMSFRKALEQKLYGLQADITISLDGVPFLMHDTTLRRTTNVEEEFPELARRPASMLNWTTLQRLNAGQWFLKTDPFWTASSLSPSDHREAQNQSICSLAELLELAKGNATLLLNLRDPPREHPYRSSFINVTLEAVLHSGFPQHQVMWLPSRQRPLVRKVAPGFQQTSGSKEAVASLRRGHIQRLNLRYTQVSRQELRDYASWNLSVNLYTVNAPWLFSLLWCAGVPSVTSDNSHALSQVPSPLWIMPPDEYCLMWVTADLVSFTLIVGIFVLQKWRLGGIRSYNPEQIMLSAAVRRTSRDVSIMKEKLIFSEISDGVEVSDVLSVCSDNSYDTYANSTATPVGPRGGGSHTKTLIERSGR

SEQ ID NO: 6 - protein, 822 aa, Homo sapiens
MKPALLPWALLLLATALGPGPGPTADAQESCSMRCGALDGPCSCHPTCSGLGTCCLDFRDFCLEILPYSGSMMGGKDFVVRHFKMSSPTDASVICRFKDSIQTLGHVDSSGQVHCVSPLLYESGRIPFTVSLDNGHSFPRAGTWLAVHPNKVSMMEKSELVNETRWQYYGTANTSGNLSLTWHVKSLPTQTITIELWGYEETGMPYSQEWTAKWSYLYPLATHIPNSGSFTFTPKPAPPSYQRWRVGALRIIDSKNYAGQKDVQALWTNDHALAWHLSDDFREDPVAWARTQCQAWEELEDQLPNFLEELPDCPCTLTQARADSGRFFTDYGCDMEQGSVCTYHPGAVHCVRSVQASLRYGSGQQCCYTADGTQLLTADSSGGSTPDRGHDWGAPPFRTPPRVPSMSHWLYDVLSFYYCCLWAPDCPRYMQRRPSNDCRNYRPPRLASAFGDPHFVTFDGTNFTFNGRGEYVLLEAALTDLRVQARAQPGTMSNGTETRGTGLTAVAVQEGNSDVVEVRLANRTGGLEVLLNQEVLSFTEQSWMDLKGMFLSVAAGDRVSIMLASGAGLEVSVQGPFLSVSVLLPEKFLTHTHGLLGTLNNDPTDDFTLHSGRVLPPGTSPQELFLFGANWTVHNASSLLTYDSWFLVHNFLYQPKHDPTFEPLFPSETTLNPSLAQEAAKLCGDDHFCNFDVAATGSLSTGTATRVAHQLHQRRMQSLQPVVSCGWLAPPPNGQKEGNRYLAGSTIYFHCDNGYSLAGAETSTCQADGTWSSPTPKCQPGRSYAVLLGIIFGGLAVVAAVALVYVLLRRRKGNTHVWGAQP

SEQ ID NO: 7 - protein, 327 aa, Homo sapiens
MVRCDRGLQMLLTTAGAFAAFSLMAIAIGTDYWLYSSAHICNGTNLTMDDGPPPRRARGDLTHSGLWRVCCIEGIYKGHCFRINHFPEDNDYDHDSSEYLLRIVRASSVFPILSTILLLLGGLCIGAGRIYSRKNNIVLSAGILFVAAGLSNIIGIIVYISSNTGDPSDKRDEDKKNHYNYGWSFYFGALSFIVAETVGVLAVNIYIEKNKELRFKTKREFLKASSSSPYARMPSYRYRRRRSRSSSRSTEASPSRDVSPMGLKITGAIPMGELSMYTLSREPLKVTTAASYSPDQEASFLQVHDFFQQDLKEGFHVSMLNRRTTPV

SEQ ID NO: 8 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 9 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 10 - protein, 813 aa, Homo sapiens
MAWPCITRACCIARFWNQLDKADIAVPLVFTKYSEATEHPGAPPQPPPPQQQAQPALAPPSARAVAIETQPAQGELDAVARATGPAPGPTGEREPAAGPGRSGPGPGLGSGSTSGPADSVMRQDYRAWKVQRPEPSCRPRSEYQPSDAPFERETQYQKDFRAWPLPRRGDHPWIPKPVQISAASQASAPILGAPKRRPQSQERWPVQAAAEAREQEAAPGGAGGLAAGKASGADERDTRRKAGPAWIVRRAEGLGHEQTPLPAAQAQVQATGPEAGRGRAAADALNRQIREEVASAVSSSYRNEFRAWTDIKPVKPIKAKPQYKPPDDKMVHETSYSAQFKGEASKPTTADNKVIDRRRIRSLYSEPFKEPPKVEKPSVQSSKPKKTSASHKPTRKAKDKQAVSGQAAKKKSAEGPSTTKPDDKEQSKEMNNKLAEAKESLAQPVSDSSKTQGPVATEPDKDQGSVVPGLLKGQGPMVQEPLKKQGSVVPGPPKDLGPMIPLPVKDQDHTVPEPLKNESPVISAPVKDQGPSVPVPPKNQSPMVPAKVKDQGSVVPESLKDQGPRIPEPVKNQAPMVPAPVKDEGPMVSASVKDQGPMVSAPVKDQGPIVPAPVKGEGPIVPAPVKDEGPMVSAPIKDQDPMVPEHPKDESAMATAPIKNQGSMVSEPVKNQGLVVSGPVKDQDVVVPEHAKVHDSAVVAPVKNQGPVVPESVKNQDPILPVLVKDQGPTVLQPPKNQGRIVPEPLKNQVPIVPVPLKDQDPLVPVPAKDQGPAVPEPLKTQGPRDPQLPTVSPLPRVMIPTAPHTEYIESSP

SEQ ID NO: 11 - protein, 296 aa, Homo sapiens
MGCRHSRLSSCKPPKKKRQEPEPEQPPRPEPHELGPLNGDTAITVQLCASEEAERHQKDITRILQQHEEEKKKWAQQVEKERELELRDRLDEQQRVLEGKNEEALQVLRASYEQEKEALTHSFREASSTQQETIDRLTSQLEAFQAKMKRVEESILSRNYKKHIQDYGSPSQFWEQELESLHFVIEMKNERIHELDRRLILMETVKEKNLILEEKITTLQQENEDLHVRSRNQVVLSRQLSEDLLLTREALEKEVQLRRQLQQEKEELLYRVLGANASPAFPLAPVTPTEVSFLAT

SEQ ID NO: 12 - protein, 478 aa, Homo sapiens
MDTSDLFASCRKGDVGRVRYLLEQRDVEVNVRDKWDSTPLYYACLCGHEELVLYLLANGARCEANTFDGERCLYGALSDPIRRALRDYKQVTASCRRRDYYDDFLQRLLEQGIHSDVVFVVHGKPFRVHRCVLGARSAYFANMLDTKWKGKSVVVLRHPLINPVAFGALLQYLYTGRLDIGVEHVSDCERLAKQCQLWDLLSDLEAKCEKVSEFVASKPGTCVKVLTIEPPPADPRLREDMALLADCALPPELRGDLWELPFPCPDGFNSCPDICFRVAGCSFLCHKAFFCGRSDYFRALLDDHFRESEEPATSGGPPAVTLHGISPDVFTHVLYYMYSDHTELSPEAAYDVLSVADMYLLPGLKRLCGRSLAQMLDEDTVVGVWRVAKLFRLARLEDQCTEYMAKVIEKLVEREDFVEAVKEEAAAVAARQETDSIPLVDDIRFHVASTVQTYSAIEEAQQRLRALEDLLVSIGLDC

SEQ ID NO: 13 - protein, 211 aa, Homo sapiens
MQRTGGGAPRPGRNHGLPGSLRQPDPVALLMLLVDADQPEPMRSGARELALFLTPEPGAEAKEVEETIEGMLLRLEEFCSLADLIRSDTSQILEENIPVLKAKLTEMRGIYAKVDRLEAFVKMVGHHVAFLEADVLQAERDHGAFPQALRRWLGSAGLPSFRNVECSGTIPARCNLRLPGSSDSPASASQVAGITEVTCTGARDVRAAHTV

SEQ ID NO: 14 - protein, 357 aa, Homo sapiens
MGTIQEAGKKTDVGVREIAEAPELGAALRHGELELKEEWQDEEFPRLLPEEAGTSEDPEDPKGDSQAAAGTPSTLALCGQRPMRKRLSAPELRLSLTKGPGNDGASPTQSAPSSPDGSSDLEIDELETPSDSEQLDSGHEFEWEDELPRAEGLGTSETAERLGRGCMWDVTGEDGHHWRVFRMGPREQRVDMTVIEPYKKVLSHGGYHGDGLNAVILFASCYLPRSSIPNYTYVMEHLFRYMVGTLELLVAENYLLVHLSGGTSRAQVPPLSWIRQCYRTLDRRLRKNLRALVVVHATWYVKAFLALLRPFISSKFTRKIRFLDSLGELAQLISLDQVHIPEAVRQLDRDLHGSGGT

SEQ ID NO: 15 - protein, 1114 aa, Homo sapiens
MLRGTMTAWRGMRPEVTLACLLLATAGCFADLNEVPQVTVQPASTVQKPGGTVILGCVVEPPRMNVTWRLNGKELNGSDDALGVLITHGTLVITALNNHTVGRYQCVARMPAGAVASVPATVTLANLQDFKLDVQHVIEVDEGNTAVIACHLPESHPKAQVRYSVKQEWLEASRGNYLIMPSGNLQIVNASQEDEGMYKCAAYNPVTQEVKTSGSSDRLRVRRSTAEAARIIYPPEAQTIIVTKGQSLILECVASGIPPPRVTWAKDGSSVTGYNKTRFLLSNLLIDTTSEEDSGTYRCMADNGVGQPGAAVILYNVQVFEPPEVTMELSQLVIPWGQSAKLTCEVRGNPPPSVLWLRNAVPLISSQRLRLSRRALRVLSMGPEDEGVYQCMAENEVGSAHAVVQLRTSRPSITPRLWQDAELATGTPPVSPSKLGNPEQMLRGQPALPRPPTSVGPASPQCPGEKGQGAPAEAPIILSSPRTSKTDSYELVWRPRHEGSGRAPILYYVVKHRKVTNSSDDWTISGIPANQHRLTLTRLDPGSLYEVEMAAYNCAGEGQTAMVTFRTGRRPKPEIMASKEQQIQRDDPGASPQSSSQPDHGRLSPPEAPDRPTISTASETSVYVTWIPRGNGGFPIQSFRVEYKKLKKVGDWILATSAIPPSRLSVEITGLEKGTSYKFRVRALNMLGESEPSAPSRPYVVSGYSGRVYERPVAGPYITFTDAVNETTIMLKWMYIPASNNNTPIHGFYIYYRPTDSDNDSDYKKDMVEGDKYWHSISHLQPETSYDIKMQCFNEGGESEFSNVMICETKARKSSGQPGRLPPPTLAPPQPPLPETIERPVGTGAMVARSSDLPYLIVGVVLGSIVLIIVTFIPFCLWRAWSKQKHTTDLGFPRSALPPSCPYTMVPLGGLPGHQASGQPYLSGISGRACANGIHMNRGCPSAAVGYPGMKPQQHCPGELQQQSDTSSLLRQTHLGNGYDPQSHQITRGPKSSPDEGSFLYTLPDDSTHQLLQPHHDCCQRQEQPAAVGQSGVRRAPDSPVLEAVWDPPFHSGPPCCLGLVPVEEVDSPDSCQVSGGDWCPQHPVGAYVGQEPGMQLSPGPLVRVSFETPPLTI

SEQ ID NO: 16 - protein, 327 aa, Homo sapiens; identical to SEQ ID NO: 7

SEQ ID NO: 17 - protein, 327 aa, Homo sapiens; identical to SEQ ID NO: 7

SEQ ID NO: 18 - protein, 289 aa, Homo sapiens
MELLCHEVDPVRRAVRDRNLLRDDRVLQNLLTIEERYLPQCSYFKCVQKDIQPYMRRMVATWMLEVCEEQKCEEEVFPLAMNYLDRFLAGVPTPKSHLQLLGAVCMFLASKLKETSPLTAEKLCIYTDNSIKPQELLEWELVVLGKLKWNLAAVTPHDFIEHILRKLPQQREKLSLIRKHAQTFIALCATDFKFAMYPPSMIATGSVGAAICGLQQDEEVSSLTCDALTELLAKITNTDVDCLKACQEQIEAVLLNSLQQYRQDQRDGSKSEDELDQASTPTDVRDIDL

SEQ ID NO: 19 - protein, 421 aa, Homo sapiens
MRWILFIGALIGSSICGQEKFFGDQVLRINVRNGDEISKLSQLVNSNNLKLNFWKSPSSFNRPVDVLVPSVSLQAFKSFLRSQGLEYAVTIEDLQALLDNEDDEMQHNEGQERSSNNFNYGAYHSLEAIYHEMDNIAADFPDLARRVKIGHSFENRPMYVLKFSTGKGVRRPAVWLNAGIHSREWISQATAIWTARKIVSDYQRDPAITSILEKMDIFLLPVANPDGYVYTQTQNRLWRKTRSRNPGSSCIGADPNRNWNASFAGKGASDNPCSEVYHGPHANSEVEVKSVVDFIQKHGNFKGFIDLHSYSQLLMYPYGYSVKKAPDAEELDKVARLAAKALASVSGTEYQVGPTCTTVYPASGSSIDWAYDNGIKFAFTFELRDTGTYGFLLPANQIIPTAEETWLGLKTIMEHVRDNLY

SEQ ID NO: 20 - protein, 2017 aa, Homo sapiens
MSLGLARAQEVELTLETVIQTLESSVLCQEKGLGARDLAQDAQITSLPALIREIVTRNLSQPESPVLLPATEMASLLSLQEENQLLQQELSRVEDLLAQSRAERDELAIKYNAVSERLEQALRLEPGELETQEPRGLVRQSVELRRQLQEEQASYRRKLQAYQEGQQRQAQLVQRLQGKILQYKKRCSELEQQLLERSGELEQQRLRDTEHSQDLESALIRLEEEQQRSASLAQVNAMLREQLDQAGSANQALSEDIRKVTNDWTRCRKELEHREAAWRREEESFNAYFSNEHSRLLLLWRQVVGFRRLVSEVKMFTERDLLQLGGELARTSRAVQEAGLGLSTGLRLAESRAEAALEKQALLQAQLEEQLRDKVLREKDLAQQQMQSDLDKADLSARVTELGLAVKRLEKQNLEKDQVNKDLTEKLEALESLRLQEQAALETEDGEGLQQTLRDLAQAVLSDSESGVQLSGSERTADASNGSLRGLSGQRTPSPPRRSSPGRGRSPRRGPSPACSDSSTLALIHSALHKRQLQVQDMRGRYEASQDLLGTLRKQLSDSESERRALEEQLQRLRDKTDGAMQAHEDAQREVQRLRSANELLSREKSNLAHSLQVAQQQAEELRQEREKLQAAQEELRRQRDRLEEEQEDAVQDGARVRRELERSHRQLEQLEGKRSVLAKELVEVREALSRATLQRDMLQAEKAEVAEALTKAEAGRVELELSMTKLRAEEASLQDSLSKLSALNESLAQDKLDLNRLVAQLEEEKSALQGRQRQAEQEATVAREEQERLEELRLEQEVARQGLEGSLRVAEQAQEALEQQLPTLRHERSQLQEQLAQLSRQLSGREQELEQARREAQRQVEALERAAREKEALAKEHAGLAVQLVAAEREGRTLSEEATRLRLEKEALEGSLFEVQRQLAQLEARREQLEAEGQALLLAKETLTGELAGLRQQIIATQEKASLDKELMAQKLVQAEREAQASLREQRAAHEEDLQRLQREKEAAWRELEAERAQLQSQLQREQEELLARLEAEKEELSEEIAALQQERDEGLLLAESEKQQALSLKESEKTALSEKLMGTRHSLATISLEMERQKRDAQSRQEQDRSTVNALTSELRDLRAQREEAAAAHAQEVRRLQEQARDLGKQRDSCLREAEELRTQLRLLEDARDGLRRELLEAQRKLRESQEGREVQRQEAGELRRSLGEGAKEREALRRSNEELRSAVKKAESERISLKLANEDKEQKLALLEEARTAVGKEAGELRTGLQEVERSRLEARRELQELRRQMKMLDSENTRLGRELAELQGRLALGERAEKESRRETLGLRQRLLKGEASLEVMRQELQVAQRKLQEQEGEFRTRERRLLGSLEEARGTEKQQLDHARGLELKLEAARAEAAELGLRLSAAEGRAQGLEAELARVEVQRRAAEAQLGGLRSALRRGLGLGRAPSPAPRPVPGSPARDAPAEGSGEGLNSPSTLECSPGSQPPSPGPATSPASPDLDPEAVRGALREFLQELRSAQRERDELRTQTSALNRQLAEMEAERDSATSRARQLQKAVAESEEARRSVDGRLSGVQAELALQEESVRRSERERRATLDQVATLERSLQATESELRASQEKISKMKANETKLEGDKRRLKEVLDASESRTVKLELQRRSLEGELQRSRLGLSDREAQAQALQDRVDSLQRQVADSEVKAGTLQLTVERLNGALAKVEESEGALRDKVRGLTEALAQSSASLNSTRDKNLHLQKALTACEHDRQVLQERLDAARQALSEARKQSSSLGEQVQTLRGEVADLELQRVEAEGQLQQLREVLRQRQEGEAAALNTVQKLQDERRLLQERLGSLQRALAQLEAEKREVERSALRLEKDRVALRRTLDKVEREKLRSHEDTVRLSAEKGRLDRTLTGAELELAEAQRQIQQLEAQVVVLEQSHSPAQLEVDAQQQQLELQQEVERLRSAQAQTERTLEARERAHRQRVRGLEEQVSTLKGQLQQELRRSSAPFSPPSGPPEK

SEQ ID NO: 21 - protein, 450 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 22 - protein, 1505 aa, Homo sapiens
MLCVAGARLKRELDATATVLANRQDESEQSRKRLIEQSREFKKNTPEDLRKQVAPLLKSFQGEIDALSKRSKEAEAAFLNVYKRLIDVPDPVPALDLGQQLQLKVQRLHDIETENQKLRETLEEYNKEFAEVKNQEVTIKALKEKIREYEQTLKNQAETIALEKEQKLQNDFAEKERKLQETQMSTTSKLEEAEHKVQSLQTALEKTRTELFDLKTKYDEETTAKADEIEMIMTDLERANQRAEVAQREAETLREQLSSANHSLQLASQIQKAPDVEQAIEVLTRSSLEVELAAKEREIAQLVEDVQRLQASLTKLRENSASQISQLEQQLSAKNSTLKQLEEKLKGQADYEEVKKELNILKSMEFAPSEGAGTQDAAKPLEVLLLEKNRSLQSENAALRISNSDLSGSARRKGKDQPESRRPGSLPAPPPSQLPRNPGEQASNTNGTHQFSPAGLSQDFFSSSLASPSLPLASTGKFALNSLLQRQLMQSFYSKAMQEAGSTSMIFSTGPYSTNSISSQSPLQQSPDVNGMAPSPSQSESAGSVSEGEEMDTAEIARQVKEQLIKHNIGQRIFGHYVLGLSQGSVSEILARPKPWNKLTVRGKEPFHKMKQFLSDEQNILALRSIQGRQRENPGQSLNRLFQEVPKRRNGSEGNITTRIRASETGSDEAIKSILEQAKRELQVQKTAEPAQPSSASGSGNSDDAIRSILQQARREMEAQQAALDPALKQAPLSQSDITILTPKLLSTSPMPTVSSYPPLAISLKKPSAAPEAGASALPNPPALKKEAQDAPGLDPQGAADCAQGVLRQVKNEVGRSGAWKDHWWSAVQPERRNAASSEEAKAEETGGGKEKGSGGSGGGSQPRAERSQLQGPSSSEYWKEWPSAESPYSQSSELSLTGASRSETPQNSPLPSSPIVPMSKPTKPSVPPLTPEQYEVYMYQEVDTIELTRQVKEKLAKNGICQRIFGEKVLGLSQGSVSDMLSRPKPWSKLTQKGREPFIRMQLWLNGELGQGVLPVQGQQQGPVLHSVTSLQDPLQQGCVSSESTPKTSASCSPAPESPMSSSESVKSLTELVQQPCPPIEASKDSKPPEPSDPPASDSQPTTPLPLSGHSALSIQELVAMSPELDTYGITKRVKEVLTDNNLGQRLFGETILGLTQGSVSDLLARPKPWHKLSLKGREPFVRMQLWLNDPNNVEKLMDMKRMEKKAYMKRRHSSVSDSQPCEPPSVGTEYSQGASPQPQHQLKKPRVVLAPEEKEALKRAYQQKPYPSPKTIEDLATQLNLKTSTVINWFHNYRSRIRRELFIEEIQAGSQGQAGASDSPSARSGRAAPSSEGDSCDGVEATEGPGSADTEEPKSQGEAEREEVPRPAEQTEPPPSGTPGPDDARDDDHEGGPVEGPGPLPSPASATATAAPAAPEDAATSAAAAPGEGPAAPSSAPPPSNSSSSSAPRRPSSLQSLFGLPEAAGARDSRDNPLRKKKAANLNSIIHRLEKAASREEPIEWEF

SEQ ID NO: 23 - protein, 397 aa, Homo sapiens; identical to SEQ ID NO: 8

SEQ ID NO: 24 - protein, 504 aa, Homo sapiens
MEATGTWALLLALALLLLLTLALSGTRARGHLPPGPTPLPLLGNLLQLRPGALYSGLMRLSKKYGPVFTIYLGPWRPVVVLVGQEAVREALGGQAEEFSGRGTVAMLEGTFDGHGVFFSNGERWRQLRKFTMLALRDLGMGKREGEELIQAEARCLVETFQGTEGRPFDPSLLLAQATSNVVCSLLFGLRFSYEDKEFQAVVRAAGGTLLGVSSQGGQTYEMFSWFLRPLPGPHKQLLHHVSTLAAFTVRQVQQHQGNLDASGPARDLVDAFLLKMAQEEQNPGTEFTNKNMLMTVIYLLFAGTMTVSTTVGYTLLLLMKYPHVQKWVREELNRELGAGQAPSLGDRTRLPYTDAVLHEAQRLLALVPMGIPRTLMRTTRFRGYTLPQGTEVFPLLGSILHDPNIFKHPEEFNPDRFLDADGRFRKHEAFLPFSLGKRVCLGEGLAKAELFLFFTTILQAFSLESPCPPDTLSLKPTVSGLFNIPPAFQLQVRPTDLHSTTQTR

SEQ ID NO: 25 - protein, 631 aa, Homo sapiens
MALRKELLKSIWYAFTALDVEKSGKVSKSQLKVLSHNLYTVLHIPHDPVALEEHFRDDDDGPVSSQGYMPYLNKYILDKVEEGAFVKEHFDELCWTLTAKKNYRADSNGNSMLSNQDAFRLWCLFNFLSEDKYPLIMVPDEVEYLLKKVLSSMSLEVSLGELEELLAQEAQVAQTTGGLSVWQFLELFNSGRCLRGVGRDTLSMAIHEVYQELIQDVLKQGYLWKRGHLRRNWAERWFQLQPSCLCYFGSEECKEKRGIIPLDAHCCVEVLPDRDGKRCMFCVKTANRTYEMSASDTRQRQEWTAAIQMAIRLQAEGKTSLHKDLKQKRREQREQRERRRAAKEEELLRLQQLQEEKERKLQELELLQEAQRQAERLLQEEEERRRSQHRELQQALEGQLREAEQARASMQAEMELKEEEAARQRQRIKELEEMQQRLQEALQLEVKARRDEESVRIAQTRLLEEEEEKLKQLMQLKEEQERYIERAQQEKEELQQEMAQQSRSLQQAQQQLEEVRQNRQRADEDVEAAQRKLRQASTNVKHWNVQMNRLMHPIEPGDKRPVTSSSFSGFQPPLLAHRDSSLKRLTRWGSQGNRTPSPNSNEQQKSLNGGDEAPAPASTPQEDKLDPAPEN

SEQ ID NO: 26 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 27 - protein, 350 aa, Homo sapiens; identical to SEQ ID NO: 26

SEQ ID NO: 28 - protein, 434 aa, Homo sapiens
MAMQKIFAREILDSRGNPTVEVDLHTAKGRFRAAVPSGASTGIYEALELRDGDKGRYLGKGVLKAVENINNTLGPALLQKKLSVVDQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 29 - protein, 998 aa, Homo sapiens
MARARPPPPPSPPPGLLPLLPPLLLLPLLLLPAGCRALEETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGMKVYIDPFTYEDPNEAVREFAKEIDVSCVKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLIRNAASLKVIASAQSGMSQPLLDRTVPDYTTFTTVGDWLDAIKMGRYKESFVSAGFASFDLVAQMTAEDLLRIGVTLAGHQKKILSSIQDMRLQMNQTLPVQV

SEQ ID NO: 30 - protein, 585 aa, Homo sapiens
MDALLGTGPRRARGCLGAAGPTSSGRAARTPAAPWARFSAWLECVCVVTFDLELGQALELVYPNDFRLTDKEKSSICYLSFPDSHSGCLGDTQFSFRMRQCGGQRSPWHADDRHYNSRAPVALQREPAHYFGYVYFRQVKDSSVKRGYFQKSLVLVSRLPFVRLFQALLSLIAPEYFDKLAPCLEAVCSEIDQWPAPAPGQTLNLPVMGVVVQVRIPSRVDKSESSPPKQFDQENLLPAPVVLASVHELDLFRCFRPVLTHMQTLWELMLLGEPLLVLAPSPDVSSEMVLALTSCLQPLRFCCDFRPYFTIHDSEFKEFTTRTQAPPNVVLGVTNPFFIKTLQHWPHILRVGEPKMSGDLPKQVKLKKPSRLKTLDTKPGLYTAYTAHLHRDKALLKRLLKGVQKKRPSDVQSALLRRHLLELTQSFIIPLEHYMASLMPLQKSITPWKTPPQIQPFSQDDFLRSLEHAGPQLTCILKGDWLGLYRRFFKSPHFDGWYRQRHKEMALKLEALHLEAICEANIETWMKDKSEVEVVDLVLKLREKLVRAQGHQLPVKEATLQRAQLYIETVIGSLPKDLQAVLCPP

SEQ ID NO: 31 - protein, 425 aa, Homo sapiens
MMPSESGAERRDRAAAQVGTAAATAVATAAPAGGGPDPEALSAFPGRHLSGLSWPQVKRLDALLSEPIPIHGRGNFPTLSVQPRQIVQVVRSTLEEQGLHVHSVRLHGSAASHVLHPESGLGYKDLDLVFRVDLRSEASFQLTKAVVLACLLDFLPAGVSRAKITPLTLKEAYVQKLVKVCTDSDRWSLISLSNKSGKNVELKFVDSVRRQFEFSIDSFQIILDSLLLFGQCSSTPMSEAFHPTVTGESLYGDFTEALEHLRHRVIATRSPEEIRGGGLLKYCHLLVRGFRPRPSTDVRALQRYMCSRFFIDFPDLVEQRRTLERYLEAHFGGADAARRYACLVTLHRVVNESTVCLMNHERRQTLDLIAALALQALAEQGPAATAALAWRPPGTDGVVPATVNYYVTPVQPLLAHAYPTWLPCN

SEQ ID NO: 32 - protein, 889 aa, Homo sapiens
MSYFGEHFWGEKNHGFEVLYHSVKQGPISTKELADFIRERATIEETYSKAMAKLSKLASNGTPMGTFAPLWEVFRVSSDKLALCHLELTRKLQDLIKDVLRYGEEQLKTHKKCKEEVVSTLDAVQVLSGVSQLLPKSRENYLNRCMDQERLRRESTSQKEMDKAETKTKKAAESLRRSVEKYNSARADFEQKMLDSALRFQAMEETHLRHMKALLGSYAHSVEDTHVQIGQVHEEFKQNIENVSVEMLLRKFAESKGTGREKPGPLDFEAYSAAALQEAMKRLRGAKAFRLPGLSRREREPEPPAAVDFLEPDSGTCPEVDEEGFTVRPDVTQNSTAEPSRFSSSDSDFDDEEPRKFYVHIKPAPARAPACSPEAAAAQLRATAGSLILPPGPGGTMKRHSSRDAAGKPQRPRSAPRTSSCAERLQSEEQVSKNLFGPPLESAFDHEDFTGSSSLGFTSSPSPFSSSSPENVEDSGLDSPSHAAPGPSPDSWVPRPGTPQSPPSCRAPPPEARGIRAPPLPDSPQPLASSPGPWGLEALAGGDLMPAPADPTAREGLAAPPRRLRSRKVSCPLTRSNGDLSRSLSPSPLGSSAASTALERPSFLSQTGHGVSRGPSPVVLGSQDALPIATAFTEYVHAYFRGHSPSCLARVTGELTMTFPAGIVRVFSGTPPPPVLSFRLVHTTAIEHFQPNADLLFSDPSQSDPETKDFWLNMAALTEALQRQAEQNPTASYYNVVLLRYQFSRPGPQSVPLQLSAHWQCGATLTQVSVEYGYRPGATAVPTPLTNVQILLPVGEPVTNVRLQPAATWNLEEKRLTWRLPDVSEAGGSGRLSASWEPLSGPSTPSPVAAQFTSEGTTLSGVDLELVGSGYRMSLVKRRFATGMYLVSC

SEQ ID NO: 33 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 34 - protein, 461 aa, Homo sapiens; identical to SEQ ID NO: 4

SEQ ID NO: 35 - protein, 565 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 36 - protein, 605 aa, Homo sapiens; identical to SEQ ID NO: 5

SEQ ID NO: 37 - protein, 693 aa, Homo sapiens; identical to SEQ ID NO: 3

SEQ ID NO: 38 - protein, 347 aa, Homo sapiens
MLHTTQLYQHVPETRWPIVYSPRYNITFMGLEKLHPFDAGKWGKVINFLKEEKLLSDSMLVEAREASEEDLLVVHTRRYLNELKWSFAVATITEIPPVIFLPNFLVQRKVLRPLRTQTGGTIMAGKLAVERGWAINVGGGFHHCSSDRGGGFCAYADITLAIKFLFERVEGISRATIIDLDAHQGNGHERDFMDDKRVYIMDVYNRHIYPGDRFAKQAIRRKVELEWGTEDDEYLDKVERNIKKSLQEHLPDVVVYNAGTDILEGDRLGGLSISPAGIVKRDELVFRMVRGRRVPILMVTSGGYQKRTARIIADSILNLFGLGLIGPESPSVSAQNSDTPLLPPAVP

SEQ ID NO: 39 - protein, 244 aa, Homo sapiens
MMRTQCLLGLRTFVAFAAKLWSFFIYLLRRQIRTVIQYQTVRYDILPLSPVSRNRLAQVKRKILVLDLDETLIHSHHDGVLRPTVRPGTPPDFILKVVIDKHPVRFFVHKRPHVDFFLEVVSQWYELVVFTASMEIYGSAVADKLDNSRSILKRRYYRQHCTLELGSYIKDLSVVHSDLSSIVILDNSPGAYRSHPDNAIPIKSWFSDPSDTALLNLLPMLDALRFTADVRSVLSRNLHQHRLW

SEQ ID NO: 40 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 41 - protein, 404 aa, Homo sapiens
MECALDAQSLISISLRKIHSSRTQRGGIKLHKNLLVSYVLRNARQLYLSERYAELYRRQQQQQQQQPPHHQHQHLAYAAPGMPASAADFGPLQLGGGGDAEAREPAARHQLHQLHQLHQLHLQQQLHQHQHPAPRGCAAAAAAGAPAGGAGALSELPGCAALQPPHGAPHRGQPLEPLQPGPAPLPLPLPPPAPAALCPRDPRAPAACSAPPGAAPPAAAASPPASPAPASSPGFYRGAYPTPSDFGLHCSSQTTVLDLDTHVVTTVENGYLHQDCCASAHCPCCGQGAPGPGLASAAGCKRKYYPGQEEEEDDEEDAGGLGAEPPGGAPFAPCKRARFEDFCPDSSPDASNISNLISIFGSGFSGLVSRQPDSSEQPPPLNGQLCAKQALASLGAWTRAIVAF

SEQ ID NO: 42 - protein, 272 aa, Homo sapiens; identical to SEQ ID NO: 9

SEQ ID NO: 43 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 44 - protein, 469 aa, Homo sapiens
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 45 - protein, 3695 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 46 - protein, 638 aa, Homo sapiens
MSALAGEDVWRCPGCGDHIAPSQIWYRTVNETWHGSCFRCSECQDSLTNWYYEKDGKLYCPKDYWGKFGEFCHGCSLLMTGPFMVAGEFKYHPECFACMSCKVIIEDGDAYALVQHATLYCGKCHNEVVLAPMFERLSTESVQEQLPYSVTLISMPATTEGRRGFSVSVESACSNYATTVQVKEVNRMHISPNNRNAIHPGDRILEINGTPVRTLRVEEVEDAISQTSQTLQLLIEHDPVSQRLDQLRLEARLAPHMQNAGHPHALSTLDTKENLEGTLRRRSLRRSNSISKSPGPSSPKEPLLFSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSMDPFPWQQKVRFAKGIASGMAYLHSMCIIHRDLNSHNCLIKLDKTVVVADFGLSRLIVEERKRAPMEKATTKKRTLRKNDRKKRYTVVGNPYWMAPEMLNGKSYDETVDIFSFGIVLCEIIGQVYADPDCLPRTLDFGLNVKLFWEKFVPTDCPPAFFPLAAICCRLEPESRPAFSKLEDSFEALSLYLGELGIPLPAELEELDHTVSMQYGLTRDSPP

SEQ ID NO: 47 - protein, 574 aa, Homo sapiens
MALARGSRQLGALVWGACLCVLVHGQQAQPGQGSDPARWRQLIQWENNGQVYSLLNSGSEYVPAGPQRSESSSRVLLAGAPQAQQRRSHGSPRRRQAPSLPLPGRVGSDTVRGQARHPFGFGQVPDNWREVAVGDSTGMARARTSVSQQRHGGSASSVSASAFASTYRQQPSYPQQFPYPQAPFVSQYENYDPASRTYDQGFVYYRPAGGGVGAGAAAVASAGVIYPYQPRARYEEYGGGEELPEYPPQGFYPAPERPYVPPPPPPPDGLDRRYSHSLYSEGTPGFEQAYPDPGPEAAQAHGGDPRLGWYPPYANPPPEAYGPPRALEPPYLPVRSSDTPPPGGERNGAQQGRLSVGSVYRPNQNGRGLPDLVPDPNYVQASTYVQRAHLYSLRCAAEEKCLASTAYAPEATDYDVRVLLRFPQRVKNQGTADFLPNRPRHTWEWHSCHQHYHSMDEFSHYDLLDAATGKKVAEGHKASFCLEDSTCDFGNLKRYACTSHTQGLSPGCYDTYNADIDCQWIDITDVQPGNYILKVHVNPKYIVLESDFTNNVVRCNIHYTGRYVSATNCKIVQS

SEQ ID NO: 48 - protein, 774 aa, Homo sapiens
MERPLCSHLCSCLAMLALLSPLSLAQYDSWPHYPEYFQQPAPEYHQPQAPANVAKIQLRLAGQKRKHSEGRVEVYYDGQWGTVCDDDFSIHAAHVVCRELGYVEAKSWTASSSYGKGEGPIWLDNLHCTGNEATLAACTSNGWGVTDCKHTEDVGVVCSDKRIPGFKFDNSLINQIENLNIQVEDIRIRAILSTYRKRTPVMEGYVEVKEGKTWKQICDKHWTAKNSRVVCGMFGFPGERTYNTKVYKMFASRRKQRYWPFSMDCTGTEAHISSCKLGPQVSLDPMKNVTCENGLPAVVSCVPGQVFSPDGPSRFRKAYKPEQPLVRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACSETAPDLVLNAEMVQQTTYLEDRPMFMLQCAMEENCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITMGCWDMYRHDIDCQWVDITDVPPGDYLFQVVINPNFEVAESDYSNNIMKCRSRYDGHRIWMYNCHIGGSFSEETEKKFEHFSGLLNNQLSPQ

SEQ ID NO: 49 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 50 - protein, 813 aa, Homo sapiens; identical to SEQ ID NO: 10

SEQ ID NO: 51 - protein, 154 aa, Homo sapiens
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 52 - protein, 445 aa, Homo sapiens
MLKKQSAGLVLWGAILFVAWNALLLLFFWTRPAPGRPPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 53 - protein, 1136 aa, Homo sapiens
MAKMEVKTSLLDNMIGVGDMVLLEPLNEETFINNLKKRFDHSEIYTYIGSVVISVNPYRSLPIYSPEKVEEYRNRNFYELSPHIFALSDEAYRSLRDQDKDQCILITGESGAGKTEASKLVMSYVAAVCGKGAEVNQVKEQLLQSNPVLEAFGNAKTVRNDNSSRFGKYMDIEFDFKGDPLGGVISNYLLEKSRVVKQPRGERNFHVFYQLLSGASEELLNKLKLERDFSRYNYLSLDSAKVNGVDDAANFRTVRNAMQIVGFMDHEAESVLAVVAAVLKLGNIEFKPESRVNGLDESKIKDKNELKEICELTGIDQSVLERAFSFRTVEAKQEKVSTTLNVAQAYYARDALAKNLYSRLFSWLVNRINESIKAQTKVRKKVMGVLDIYGFEIFEDNSFEQFIINYCNEKLQQIFIELTLKEEQEEYIREDIEWTHIDYFNNAIICDLIENNTNGILAMLDEECLRPGTVTDETFLEKLNQVCATHQHFESRMSKCSRFLNDTSLPHSCFRIQHYAGKVLYQVEGFVDKNNDLLYRDLSQAMWKASHALIKSLFPEGNPAKINLKRPPTAGSQFKASVATLMKNLQTKNPNYIRCIKPNDKKAAHIFNEALVCHQIRYLGLLENVRVRRAGYAFRQAYEPCLERYKMLCKQTWPHWKGPARSGVEVLFNELEIPVEEYSFGRSKIFIRNPRTLFKLEDLRKQRLEDLATLIQKIYRGWKCRTHFLLMKKSQIVIAAWYRRYAQQKRYQQTKSSALVIQSYIRGWKARKILRELKHQKRCKEAVTTIAAYWHGTQARRELRRLKEEARNKHAIAVIWAYWLGSKARRELKRLKEEARRKHAVAVIWAYWLGLKVRREYRKFFRANAGKKIYEFTLQRIVQKYFLEMKNKMPSLSPIDKNWPSRPYLFLDSTHKELKRIFHLWRCKKYRDQFTDQQKLIYEEKLEASELFKDKKALYPSSVGQPFQGAYLEINKNPKYKKLKDAIEEKIIIAEVVNKINRANGKSTSRIFLLTNNNLLLADQKSGQIKSEVPLVDVTKVSMSSQNDGFFAVHLKEGSEAASKGDFLFSSDHLIEMATKLYRTTLSQTKQKLNIEISDEFLVQFRQDKVCVKFIQGNQKNGSVPTCKRKNNRLLEVAVP

SEQ ID NO: 54 - protein, 525 aa, Homo sapiens
MHRAPSPTAEQPPGGGDSARRTLQPRLKPSARAMALPRTLGELQLYRVLQRANLLSYYETFIQQGGDDVQQLCEAGEEEFLEIMALVGMATKPLHVRRLQKALREWATNPGLFSQPVPAVPVSSIPLFKISETAGTRKGSMSNGHGSPGEKAGSARSFSPKSPLELGEKLSPLPGGPGAGDPRIWPGRSTPESDVGAGGEEEAGSPPFSPPAGGGVPEGTGAGGLAAGGTGGGPDRLEPEMVRMVVESVERIFRSFPRGDAGEVTSLLKLNKKLARSVGHIFEMDDNDSQKEEEIRKYSIIYGRFDSKRREGKQLSLHELTINEAAAQFCMRDNTLLLRRVELFSLSRQVARESTYLSSLKGSRLHPEELGGPPLKKLKQEVGEQSHPEIQQPPPGPESYVPPYRPSLEEDSASLSGESLDGHLQAVGSCPRLTPPPADLPLALPAHGLWSRHILQQTLMDEGLRLARLVSHDRVGRLSPCVPAKPPLAEFEEGLLDRCPAPGPHPALVEGRRSSVKVEAEASRQ

SEQ ID NO: 55 - protein, 1060 aa, Homo sapiens
MDSGAGGRRCPEAALLILGPPRMEHLRHSPGPGGQRLLLPSMLLALLLLLAPSPGHATRVVYKVPEEQPPNTLIGSLAADYGFPDVGHLYKLEVGAPYLRVDGKTGDIFTTETSIDREGLRECQNQLPGDPCILEFEVSITDLVQNGSPRLLEGQIEVQDINDNTPNFASPVITLAIPENTNIGSLFPIPLASDRDAGPNGVASYELQAGPEAQELFGLQVAEDQEEKQPQLIVMGNLDRERWDSYDLTIKVQDGGSPPRASSALLRVTVLDTNDNAPKFERPSYEAELSENSPIGHSVIQVKANDSDQGANAEIEYTFHQAPEVVRRLLRLDRNTGLITVQGPVDREDLSTLRFSVLAKDRGTNPKSARAQVVVTVKDMNDNAPTIEIRGIGLVTHQDGMANISEDVAEETAVALVQVSDRDEGENAAVTCVVAGDVPFQLRQASETGSDSKKKYFLQTTTPLDYEKVKDYTIEIVAVDSGNPPLSSTNSLKVQVVDVNDNAPVFTQSVTEVAFPENNKPGEVIAEITASDADSGSNAELVYSLEPEPAAKGLFTISPETGEIQVKTSLDREQRESYELKVVAADRGSPSLQGTATVLVNVLDCNDNDPKFMLSGYNFSVMENMPALSPVGMVTVIDGDKGENAQVQLSVEQDNGDFVIQNGTGTILSSLSFDREQQSTYTFQLKAVDGGVPPRSAYVGVTINVLDENDNAPYITAPSNTSHKLLTPQTRLGETVSQVAAEDFDSGVNAELIYSIAGGNPYGLFQIGSHSGAITLEKEIERRHHGLHRLVVKVSDRGKPPRYGTALVHLYVNETLANRTLLETLLGHSLDTPLDIDIAGDPEYERSKQRGNILFGVVAGVVAVALLIALAVLVRYCRQREAKSGYQAGKKETKDLYAPKPSGKASKGNKSKGKKSKSPKPVKPVEDEDEAGLQKSLKFNLMSDAPGDSPRIHLPLNYPPGSPDLGRHYRSNSPLPSIQLQPQSPSASKKHQVVQDLPPANTFVGTGDTTSTGSEQYSDYSYRTNPPKYPSKQVGQPFQLSTPQPLPHPYHGAIWTEVWE

SEQ ID NO: 56 - protein, 329 aa, Homo sapiens
MTTQQIDLQGPGPWGFRLVGGKDFEQPLAISRVTPGSKAALANLCIGDVITAIDGENTSNMTHLEAQNRIKGCTDNLTLTVARSEHKVWSPLVTEEGKRHPYKMNLASEPQEVLHIGSAHNRSAMPFTASPASSTTARVITNQYNNPAGLYSSENISNFNNALESKTAASGVEANSRPLDHAQPPSSLVIDKESEVYKMLQEKQELNEPPKQSTSFLVLQEILESEEKGDPNKPSGFRSVKAPVTKVAASIGNAQKLPMCDKCGTGIVGVFVKLRDRHRHPECYVCTDCGTNLKQKGHFFVEDQIYCEKHARERVTPPEGYEVVTVFPK

SEQ ID NO: 57 - protein, 806 aa, Homo sapiens
MQFFGRLVNTFSGVTNLFSNPFRVKEVAVADYTSSDRVREEGQLILFQNTPNRTWDCVLVNPRNSQSGFRLFQLELEADALVNFHQYSSQLLPFYESSPQVLHTEVLQHLTDLIRNHPSWSVAHLAVELGIRECFHHSRIISCANCAENEEGCTPLHLACRKGDGEILVELVQYCHTQMDVTDYKGETVFHYAVQGDNSQVLQLLGRNAVAGLNQVNNQGLTPLHLACQLGKQEMVRVLLLCNARCNIMGPNGYPIHSAMKFSQKGCAEMIISMDSSQIHSKDPRYGASPLHWAKNAEMARMLLKRGCNVNSTSSAGNTALHVAVMRNRFDCAIVLLTHGANADARGEHGNTPLHLAMSKDNVEMIKALIVFGAEVDTPNDFGETPTFLASKIGRLVTRKAILTLLRTVGAEYCFPPIHGVPAEQGSAAPHHPFSLERAQPPPISLNNLELQDLMHISRARKPAFILGSMRDEKRTHDHLLCLDGGGVKGLIIIQLLIAIEKASGVATKDLFDWVAGTSTGGILALAILHSKSMAYMRGMYFRMKDEVFRGSRPYESGPLEEFLKREFGEHTKMTDVRKPKVMLTGTLSDRQPAELHLFRNYDAPETVREPRFNQNVNLRPPAQPSDQLVWRAARSSGAAPTYFRPNGRFLDGGLLANNPTLDAMTEIHEYNQDLIRKGQANKVKKLSIVVSLGTGRSPQVPVTCVDVFRPSNPWELAKTVFGAKELGKMVVDCCTDPDGRAVDRARAWCEMVGIQYFRLNPQLGTDIMLDEVSDTVLVNALWETEVYIYEHREEFQKLIQLLLSP

SEQ ID NO: 58 - protein, 1659 aa, Homo sapiens
MEAPSGSEPGGDGAGDCAHPDPRAPGAAAPSSGPGPCAAARESERQLRLRLCVLNEILGTERDYVGTLRFLQSAFLHRIRQNVADSVEKGLTEENVKVLFSNIEDILEVHKDFLAALEYCLHPEPQSQHELGNVFLKFKDKFCVYEEYCSNHEKALRLLVELNKIPTVRAFLLSCMLLGGRKTTDIPLEGYLLSPIQRICKYPLLLKELAKRTPGKHPDHPAVQSALQAMKTVCSNINETKRQMEKLEALEQLQSHIEGWEGSNLTDICTQLLLQGTLLKISAGNIQERAFFLFDNLLVYCKRKSRVTGSKKSTKRTKSINGSLYIFRGRINTEVMEVENVEDGTADYHSNGYTVTNGWKIHNTAKNKWFVCMAKTAEEKQKWLDAIIREREQRESLKLGMERDAYVMIAEKGEKLYHMMMNKKVNLIKDRRRKLSTVPKCFLGNEFVAWLLEIGEISKTEEGVNLGQALLENGIIHHVSDKHQFKNEQVMYRFRYDDGTYKARSELEDIMSKGVRLYCRLHSLYTPVIKDRDYHLKTYKSVLPGSKLVDWLLAQGDCQTREEAVALGVGLCNNGFMHHVLEKSEFRDESQYFRFHADEEMEGTSSKNKQLRNDFKLVENILAKRLLILPQEEDYGFDIEEKNKAVVVKSVQRGSLAEVAGLQVGRKIYSINEDLVFLRPFSEVESILNQSFCSRRPLRLLVATKAKEIIKIPDQPDTLCFQIRGAAPPYVYAVGRGSEAMAAGLCAGQCILKVNGSNVMNDGAPEVLEHFQAFRSRREEALGLYQWIYHTHEDAQEARASQEASTEDPSGEQAQEEDQADSAFPLLSLGPRLSLCEDSPMVTLTVDNVHLEHGVVYEYVSTAGVRCHVLEKIVEPRGCFGLTAKILEAFAANDSVFVENCRRLMALSSAIVTMPHFEFRNICDTKLESIGQRIACYQEFAAQLKSRVSPPFKQAPLEPHPLCGLDFCPTNCHINLMEVSYPKTTPSVGRSFSIRFGRKPSLIGLDPEQGHLNPMSYTQHCITTMAAPSWKCLPAAEGDPQGQGLHDGSFGPASGTLGQEDRGLSFLLKQEDREIQDAYLQLFTKLDVALKEMKQYVTQINRLLSTITEPTSGGSCDASLAEEASSLPLVSEESEMDRSDHGGIKKVCFKVAEEDQEDSGHDTMSYRDSYSECNSNRDSVLSYTSVRSNSSYLGSDEMGSGDELPCDMRIPSDKQDKLHGCLEHLFNQVDSINALLKGPVMSRAFEETKHFPMNHSLQEFKQKEECTIRGRSLIQISIQEDPWNLPNSIKTLVDNIQRYVEDGKNQLLLALLKCTDTELQLRRDAIFCQALVAAVCTFSKQLLAALGYRYNNNGEYEESSRDASRKWLEQVAATGVLLHCQSLLSPATVKEERTMLEDIWVTLSELDNVTFSFKQLDENYVANTNVFYHIEGSRQALKVIFYLDSYHFSKLPSRLEGGASLRLHTALFTKVLENVEGLPSPGSQAAEDLQQDINAQSLEKVQQYYRKLRAFYLERSNLPTDASTTAVKIDQLIRPINALDELCRLMKSFVHPKPGAAGSVGAGLIPISSELCYRLGACQMVMCGTGMQRSTLSVSLEQAAILARSHGLLPKCIMQATDIMRKQGPRVEILAKNLRVKDQMPQGAPRLYRLCQPPVDGDL

SEQ ID NO: 59 - protein, 871 aa, Homo sapiens
MMPPPFMPPPGIPPPFPPMGLPPMSQRPPAIPPMPPGILPPMLPPMGAPPPLTQIPGMVPPMMPGMLMPAVPVTAATAPGADTASSAVAGTGPPRALWSEHVAPDGRIYYYNADDKQSVWEKPSVLKSKAELLLSQCPWKEYKSDTGKPYYYNNQSKESRWTRPKDLDDLEVLVKQEAAGKQQQQLPQTLQPQPPQPQPDPPPVPPGPTPVPTGLLEPEPGGSEDCDVLEATQPLEQGFLQQLEEGPSSSGQHQPQQEEEESKPEPERSGLSWSNREKAKQAFKELLRDKAVPSNASWEQAMKMVVTDPRYSALPKLSEKKQAFNAYKAQREKEEKEEARLRAKEAKQTLQHFLEQHERMTSTTRYRRAEQTFGELEVWAVVPERDRKEVYDDVLFFLAKKEKEQAKQLRRRNIQALKSILDGMSSVNFQTTWSQAQQYLMDNPSFAQDHQLQNMDKEDALICFEEHIRALEREEEEERERARLRERRQQRKNREAFQTFLDELHETGQLHSMSTWMELYPAVSTDVRFANMLGQPGSTPLDLFKFYVEELKARFHDEKKIIKDILKDRGFCVEVNTAFEDFAHVISFDKRAAALDAGNIKLTFNSLLEKAEAREREREKEEARRMRRREAAFRSMLRQAVPALELGTAWEEVRERFVCDSAFEQITLESERIRLFREFLQVLEQTECQHLHTKGRKHGRKGKKHHHKRSHSPSGSESEEEELPPPSLRPPKRRRRNPSESGSEPSSSLDSVESGGAALGGRGSPSSHLLGADHGLRKAKKPKKKTKKRRHKSNSPESETDPEEKAGKESDEKEQEQDKDRELQQAELPNRSPGFGIKKEKTGWDTSESELSEGELERRRRTLLQQLDDHQ

SEQ ID NO: 60 - protein, 567 aa, Homo sapiens
MHGRLKVKTSEEQAEAKRLEREQKLKLYQSATQAVFQKRQAGELDESVLELTSQILGANPDFATLWNCRREVLQQLETQKSPEELAALVKAELGFLESCLRVNPKSYGTWHHRCWLLGRLPEPNWTRELELCARFLEVDERNFHCWDYRRFVATQAAVPPAEELAFTDSLITRNFSNYSSWHYRSCLLPQLHPQPDSGPQGRLPEDVLLKELELVQNAFFTDPNDQSAWFYHRWLLGRADPQDALRCLHVSRDEACLTVSFSRPLLVGSRMEILLLMVDDSPLIVEWRTPDGRNRPSHVWLCDLPAASLNDQLPQHTFRVIWTAGDVQKECVLLKGRQEGWCRDSTTDEQLFRCELSVEKSTVLQSELESCKELQELEPENKWCLLTIILLMRALDPLLYEKETLQYFQTLKAVDPMRATYLDDLRSKFLLENSVLKMEYAEVRVLHLAHKDLTVLCHLEQLLLVTHLDLSHNRLRTLPPALAALRCLEVLQASDNAIESLDGVTNLPRLQELLLCNNRLQQPAVLQPLASCPRLVLLNLQGNPLCQAVGILEQLAELLPSVSSVLT

SEQ ID NO: 61 - protein, 90 aa, Homo sapiens
MACPLDQAIGLLVAIFHKYSGREGDKHTLSKKELKELIQKELTIGSKLQDAEIARLMEDLDRNKDQEVNFQEYVTFLGALALIYNEALKG

SEQ ID NO: 62 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 63 - protein, 583 aa, Homo sapiens
MDLLPPKPKYNPLRNESLSSLEEGASGSTPPEELPSPSASSLGPILPPLPGDDSPTTLCSFFPRMSNLRLANPAGGRPGSKGEPGRAADDGEGIVGAAMPDSGPLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 64 - protein, 707 aa, Homo sapiens
MAAAATAAEGVPSRGPPGEVIHLNVGGKRFSTSRQTLTWIPDSFFSSLLSGRISTLKDETGAIFIDRDPTVFAPILNFLRTKELDPRGVHGSSLLHEAQFYGLTPLVRRLQLREELDRSSCGNVLFNGYLPPPVFPVKRRNRHSLVGPQQLGGRPAPVRRSNTMPPNLGNAGLLGRMLDEKTPPSPSGQPEEPGMVRLVCGHHNWIAVAYTQFLVCYRLKEASGWQLVFSSPRLDWPIERLALTARVHGGALGEHDKMVAAATGSEILLWALQAEGGGSEIGVFHLGVPVEALFFVGNQLIATSHTGRIGVWNAVTKHWQVQEVQPITSYDAAGSFLLLGCNNGSIYYVDVQKFPLRMKDNDLLVSELYRDPAEDGVTALSVYLTPKTSDSGNWIEIAYGTSSGGVRVIVQHPETVGSGPQLFQTFTVHRSPVTKIMLSEKHLISVCADNNHVRTWSVTRFRGMISTQPGSTPLASFKILALESADGHGGCSAGNDIGPYGERDDQQVFIQKVVPSASQLFVRLSSTGQRVCSVRSVDGSPTTAFTVLECEGSRRLGSRPRRYLLTGQANGSLAMWDLTTAMDGLGQAPAGGLTEQELMEQLEHCELAPPAPSAPSWGCLPSPSPRISLTSLHSASSNTSLSGHRGSPSPPQAEARRRGGGSFVERCQELVRSGPDLRRPPTPAPWPSSGLGTPLTPPKMKLNETSF

SEQ ID NO: 65 - protein, 494 aa, Homo sapiens
MAMSLPGSRRTSAGSRRRTSPPVSVRDAYGTSSLSSSSNSGSYKGSDSSPTPRRSMKYTLCSDNHGIKPPTPEQYLTPLQQKEVCIRHLKARLKDTQDRLQDRDTEIDDLKTQLSRMQEDWIEEECHRVEAQLALKEARKEIKQLKQVIDTVKNNLIDKDKGLQKYFVDINIQNKKLETLLHSMEVAQNGMAKEDGTGESAGGSPARSLTRSSTYTKLSDPAVCGDRQPGDPSSGSAEDGADSGFAAADDTLSRTDALEASSLLSSGVDCGTEETSLHSSFGLGPRFPASNTYEKLLCGMEAGVQASCMQERAIQTDFVQYQPDLDTILEKVTQAQVCGTDPESGDRCPELDAHPSGPRDPNSAVVVTVGDELEAPEPITRGPTPQRPGANPNPGQSVSVVCPMEEEEEAAVAEKEPKSYWSRHYIVDLLAVVVPAVPTVAWLCRSQRRQGQPIYNISSLLRGCCTVALHSIRRISCRSLSQPSPSPAGGGSQL

SEQ ID NO: 66 - protein, 822 aa, Homo sapiens; identical to SEQ ID NO: 6

SEQ ID NO: 67 - protein, 1169 aa, Homo sapiens
GLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 68 - protein, 277 aa, Homo sapiens
MASAELDYTIEIPDQPCWSQKNSPSPGGKEAETRQPVVILLGWGGCKDKNLAKYSAIYHKRGCIVIRYTAPWHMVFFSESLGIPSLRVLAQKLLELLFDYEIEKEPLLFHVFSNGGVMLYRYVLELLQTRRFCRLRVVGTIFDSAPGDSNLVGALRALAAILERRAAMLRLLLLVAFALVVVLFHVLLAPITALFHTHFYDRLQDAGSRWPELYLYSRADEVVLARDIERMVEARLARRVLARSVDFVSSAHVSHLRDYPTYYTSLCVDFMRNCVRC

SEQ ID NO: 69 - protein, 457 aa, Homo sapiens
MRPPSPLPARWLCVLAGALAWALGPAGGQAARLQEECDYVQMIEVQHKQCLEEAQLENETIGCSKMWDNLTCWPATPRGQVVVLACPLIFKLFSSIQGRNVSRSCTDEGWTHLEPGPYPIACGLDDKAASLDEQQTMFYGSVKTGYTIGYGLSLATLLVATAILSLFRKLHCTRNYIHMHLFISFILRAAAVFIKDLALFDSGESDQCSEGSVGCKAAMVFFQYCVMANFFWLLVEGLYLYTLLAVSFFSERKYFWGYILIGWGVPSTFTMVWTIARIHFEDYGCWDTINSSLWWIIKGPILTSILVNFILFICIIRILLQKLRPPDIRKSDSSPYSRLARSTLLLIPLFGVHYIMFAFFPDNFKPEVKMVFELVVGSFQGFVVAILYCFLNGEVQAELRRKWRRWHLQGVLGWNPKYRHPSGGSNGATCSTQVSMLTRVSPGARRSSSFQAEVSLV

SEQ ID NO: 70 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 71 - protein, 940 aa, Homo sapiens
MARKRAAGGEPRGRELRSQKSKAKSKARREEEEEDAFEDEKPPKKSLLSKVSQGKRKRGCSHPGGSADGPAKKKVAKVTVKSENLKVIKDEALSDGDDLRDFPSDLKKAHHLKRGATMNEDSNEEEEESENDWEEVEELSEPVLGDVRESTAFSRSLLPVKPVEIEIETPEQAKTRERSEKIKLEFETYLRRAMKRFNKGVHEDTHKVHLLCLLANGFYRNNICSQPDLHAIGLSIIPARFTRVLPRDVDTYYLSNLVKWFIGTFTVNAELSASEQDNLQTTLERRFAIYSARDDEELVHIFLLILRALQLLTRLVLSLQPIPLKSATAKGKKPSKERLTADPGGSSETSSQVLENHTKPKTSKGTKQEETFAKGTCRPSAKGKRNKGGRKKRSKPSSSEEDEGPGDKQEKATQRRPHGRERRVASRVSYKEESGSDEAGSGSDFELSSGEASDPSDEDSEPGPPKQRKAPAPQRTKAGSKSASRTHRGSHRKDPSLPAASSSSSSSKRGKKMCSDGEKAEKRSIAGIDQWLEVFCEQEEKWVCVDCVHGVVGQPLTCYKYATKPMTYVVGIDSDGWVRDVTQRYDPVWMTVTRKCRVDAEWWAETLRPYQSPFMDREKKEDLEFQAKHMDQPLPTAIGLYKNHPLYALKRHLLKYEAIYPETAAILGYCRGEAVYSRDCVHTLHSRDTWLKKARVVRLGEVPYKMVKGFSNRARKARLAEPQLREENDLGLFGYWQTEEYQPPVAVDGKVPRNEFGNVYLFLPSMMPIGCVQLNLPNLHRVARKLDIDCVQAITGFDFHGGYSHPVTDGYIVCEEFKDVLLTAWENEQAVIERKEKEKKEKRALGNWKLLAKGLLIRERLKRRYGPKSEAAAPHTDAGGGLSSDEEEGTSSQAEAARILAASWPQNREDEEKQKLKGGPKKTKREKKAAASHLFPFEQL